(19) United States
(12) Patent Application Publication
(10) Pub. No.: US 2014/0142099 A1
(43) Pub. Date: May 22, 2014

(54) Purinone Derivatives as Tyrosine Kinase Inhibitors

(71) Applicant: Principia Biopharma Inc., So. San Francisco, CA (US)

(72) Inventor: Timothy D. Owens, San Carlos, CA (US)

(73) Assignee: Principia Biopharma Inc., So. San Francisco, CA (US)

(21) Appl. No.: 14/084,519

(22) Filed: Nov. 19, 2013

Related U.S. Application Data
(60) Provisional application No. 61/728,575, filed on Nov. 20, 2012.

(51) Int. Cl. A61K 31/5377; A61K 31/522; A61K 45/06; C07D 473/34

(52) U.S. Cl. CPC A61K 31/5377; C07D 473/34; A61K 31/522; A61K 45/06
USPC 514/234.2; 544/276; 514/263.2; 544/118

(57) ABSTRACT

The present disclosure provides compounds and pharmaceutically acceptable salts thereof that are tyrosine kinase inhibitors, in particular BLK, BMX, EGFR, HER2, HER4, ITK, TEC, BTK, and TXK and are therefore useful for the treatment of diseases treatable by inhibition of tyrosine kinases such as autoimmune diseases, cancer and inflammatory diseases. Also provided are pharmaceutical compositions containing such compounds and pharmaceutically acceptable salts thereof and processes for preparing such compounds and pharmaceutically acceptable salts thereof.

This application claims the benefit of U.S. Provisional Application No. 61/728,575, filed Nov. 20, 2012, which is incorporated herein by reference.

### FIELD OF THE PRESENT DISCLOSURE

The present disclosure provides compounds that are inhibitors of tyrosine kinase such as BLK, BMX, EGFR, HER2, HER4, ITK, TEC, BTK, and TXK, in particular Bruton's tyrosine kinase (BTK), and are therefore useful for the treatment of diseases treatable by inhibition of tyrosine kinases such as cancer, autoimmune, and inflammatory diseases. Also provided are pharmaceutical compositions containing such compounds and processes for preparing such compounds.

### BACKGROUND

The human genome contains at least 500 genes encoding protein kinases. Many of these kinases have been implicated in human disease and as such represent potentially attractive therapeutic targets. BTK, a member of the Tec family non-receptor tyrosine kinases, is essential for B cell signaling downstream from the B-cell receptor. It is expressed in B cells and other hematopoietic cells such as monocytes, macrophages, and mast cells. BTK is reported to function in various aspects of B cell function that maintain the B cell repertoire (see Gauld S. B. et al., B cell antigen receptor signaling: roles in cell development and disease. Science, 296:1641-2. 2002.)). Clinical validation of the role of B cells in RA has been provided by the efficacy of the biologic Rituxan (an anti-CD20 antibody), which depletes B cells as a mechanism of action (see Perosa F., et al., CD20-depleting therapy in autoimmune diseases: from basic research to the clinic. J Intern Med. 267:260-77. 2010 and Dörner T, et al. Targeting B cells in immune-mediated inflammatory disease: a comprehensive review of mechanisms of action and identification of biomarkers. Pharmacol Ther. 125:464-75. 2010.). BTK is reported to be required for B cell development because patients with the disease X-linked agammaglobulinemia (see Rosen F. S., et al., The primary immunodeficiencies. N Engl J. Med. 333:431-40. 1995) lack of antibodies in their bloodstream. Notably, small-molecule BTK inhibitors in pre-clinical development have been reported to be efficacious in collagen-induced arthritis (see Pan Z., et al., Discovery of selective irreversible inhibitors for Bruton's tyrosine kinase. J. Med. Chem. 2:58-61. 2007). The potential advantage of a small molecule BTK inhibitor (beyond the inherent advantage of a small-molecule over a biologic) is that modulation of BTK can inhibit B cell function without permanent removal of the B cell itself. Therefore, the long periods of low B cell levels experienced with the biologic Rituxan should be avoidable by targeting BTK with a small molecule BTK inhibitor.

In addition, the disease modifying activities of BTK are expected to extend beyond those of Rituxan because of effects on addition cellular targets that are involved in propagation of disease. For instance, antigen induced mast cell degranulation is reportedly impaired in mast cells derived from the bone marrow of BTK deficient mice, demonstrating that BTK is downstream of the FcεR1 receptor (see Setoguchi R., et al., Defective degranulation and calcium mobilization of bone-marrow derived mast cells from Xid and BTK-deficient mice. Immunol Lett. 64:109-18. 1998). A similar signaling module exists in monocytes and macrophages for the FcγR1 receptor indicating BTK inhibition is highly likely to modulate TNF production in response to IgG. Both mast cells and macrophages are thought to contribute to propagation of the inflammatory cytokine environment of the diseased synovium.

In addition to the peripheral and synovial effects of BTK inhibition described above, BTK inhibition reportedly will have bone protective effects in an inflamed joint (see Gravallese E. M., et al., Synovial tissue in rheumatoid arthritis is a source of osteoclast differentiation factor. Arthritis Rheum. 43:250-8. 2000). Studies with mice that are either deficient in BTK or have impaired BTK function have reportedly demonstrated that Rank ligand-induced osteoclast differentiation is impaired in the absence of BTK function (see Lee S. H., et. al., The tec family tyrosine kinase BTK Regulates RANKL-induced osteoclast maturation. J. Biol. Chem. 283:11526-34. 2008). Taken together, these studies can be interpreted as suggesting that a BTK inhibitor could inhibit or reverse the bone destruction that occurs in RA patients. Given the importance of B cells in autoimmune disease, BTK inhibitors could also have utility in other autoimmune diseases such as systemic lupus erythematosus (see Shlomchik M. J., et. al., The role of B cells in lpr/lpr-induced autoimmunity. J. Exp Med. 180:1295-1306. 1994). Notably, an irreversible BTK inhibitor has been reported to display efficacy in the mouse MRL/lpr lupus model, reducing autoantibody production and renal damage (see Honigberg L. A., The Bruton tyrosine kinase inhibitor PCI-32765 blocks B-cell activation and is efficacious in models of autoimmune disease and B-cell malignancy. Proc. Natl. Acad. Sci. 107:13075-80. 2010).

There is also potential for BTK inhibitors for treating allergic diseases (see Honigberg, L., et. al., The selective BTK inhibitor PCI-32765 blocks B cell and mast cell activation and prevents mouse collagen indiced arthritis. Clin. Immunol. 127 S1:S111. 2008). In addition, the irreversible inhibitor reportedly suppresses passive cutaneous anaphylaxis (PCA) induced by IgE antigen complex in mice (see Honigberg, L., et. al., The selective BTK inhibitor PCI-32765 blocks B cell and mast cell activation and prevents mouse collagen indiced arthritis. Clin. Immunol. 127 S1:S111. 2008). These reported findings are in agreement with those noted with BTK-mutant mast cells and knockout mice and can be interpreted as suggesting that BTK inhibitors may be useful for the treatment of asthma, an IgE-dependent allergic disease of the airway.

In addition, platelet aggregation in response to collagen or collagen-related peptide is reportedly impaired in XLA patients who lack BTK function (see Quek L. S, et al., A role for Bruton's tyrosine kinase (BTK) in platelet activation by collagen. Curr. Biol. 8:1137-40.1998). This is manifested by changes downstream from GPIV, such as phosphorylation of PLCgamma2 and calcium flux, which can be interpreted as suggesting potential utility in treating thromboembolic diseases.

Preclinical studies with a selective inhibitor of BTK have reportedly shown effects on spontaneous canine B cell lymphomas suggesting a potential utility in human lymphomas or other hematologic malignancies including chronic lymphocytic leukemia. In addition, clinical trials with PCI-32765 can be interpreted as indicating utility for a BTK inhibitor in both chronic lymphocytic leukemia and mantle cell lymphoma (see Fowler, N et al., The Btk inhibitor, PCI-32765, induces durable responses with minimal toxicity in patients with relapsed/refractory Bcell malignancies: results from a phase I study. Blood 2010; 116 (21): 425; Byrd J. C., et al. Activity and tolerability of the Bruton's tyrosine kinase (Btk) inhibitor PCI-32765 in patients with chronic lymphocytic leukemia/small lymphocytic lymphoma (CLL/SLL): Interim results of a phase Ib/II study. J Clin Oncol 2011; 29:6508).

EGFR is reportedly overexpressed in breast, head and neck cancers, and that overexpression is correlated with poor survival (see Do N. Y., et al., Expression of c-ErbB receptors, MMPs and VEGF in squamous cell carcinoma of the head and neck. Oncol Rep. Aug. 12:229-37. 2004 and Foley J., et al., EGFR signaling in breast cancer: bad to the bone. Semin Cell Dev Biol. 21:951-60. 2010). HER2, another EGFR family member, also is reportedly amplified or overexpressed in up to 30% of breast cancers, also correlating with poor survival (see Murphy C. G, Modi S. HER2 breast cancer therapies: a review. Biologics 3:289-301. 2009). HER4, also in the EGFR family, is reportedly overexpressed in head and neck squamous cell carcinomas (see Rosen F. S., et al. The primary immunodeficiencies. New Engl. J. Med. 333:431-40. 1995). Other studies report decreased expression of HER4 in certain cancers and suggest tumor suppressor activity (see Thomasson M, et al., ErbB4 is downregulated in renal cell carcinoma—a quantitative RT-PCR and immunohistochemical analysis of the epidermal growth factor receptor family. Acta Oncol. 43:453-9. 2004). Overall the data can be interpreted to support a role for members of the EGFR family in cancer. ITK, a member of the TEC kinase family, is reportedly involved in activation of T cells and mast cells (see Iyer A. S. et al. Absence of Tec Family Kinases Interleukin-2 Inducible T cell Kinase (Itk) and Bruton's Tyrosine Kinase (Btk) Severely Impairs Fc{epsilon}RI-dependent Mast Cell Responses. J. Biol. Chem.; 286:9503-13. 2011) and is a potential target in inflammatory immune diseases such as asthma. Mice deficient in ITK are reportedly resistant to development of allergic asthma (see Sahu N, et al., Differential sensitivity to Itk kinase signals for T helper 2 cytokine production and chemokine-mediated migration. J. Immunol. 180:3833-8. 2008). Another family member, BMX, is reportedly involved in supporting tumor angiogenesis through it's role in the tumor vascular endothelium (see Tu T, et al., Bone marrow X kinase-mediated signal transduction in irradiated vascular endothelium. Cancer Res. 68:2861-9. 2008) and is also progressively up-regulated during bladder cancer progression (see Guo S., et al., Tyrosine Kinase ETK/BMX Is Up-Regulated in Bladder Cancer and Predicts Poor Prognosis in Patients with Cystectomy. PLoS One. 6:e17778. 2011), which can be interpreted to suggest a potential therapeutic target in this type cancer. The B lymphoid kinase (hereafter, sometimes expressed as “BLK”) is reportedly linked through genetic association with a variety of rheumatic diseases including systemic lupus erythematosus and systemic sclerosis (see Ito I, et al., Association of the FAM167A-BLK region with systemic sclerosis. Arthritis Rheum. 62:890-5. 2010).

Accordingly, there is a need for compounds that inhibit tyrosine kinases, and particularly BTK inhibitors, thereby providing treatment for diseases such as autoimmune diseases, inflammatory diseases, thromboembolic diseases, and cancer. The present disclosure is directed to such treatment.

### SUMMARY

In one aspect, this disclosure is directed to a compound of Formula (I):

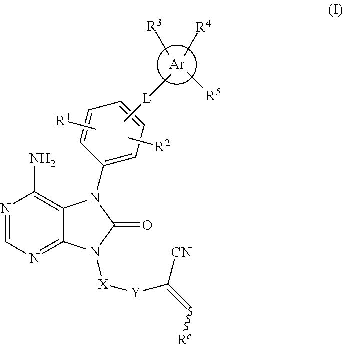

wherein:

L is O, CO, CH₂, S, SO, SO₂, NR, NRCO, CONR, or NRCONR′ where (each R and R′ is independently hydrogen or alkyl);

Ar is aryl, heteroaryl, cycloalkyl or heterocyclyl;

R¹ is hydrogen, alkyl, cyclopropyl, hydroxy, alkoxy, cyano, halo, haloalkyl or haloalkoxy;

R² is hydrogen, alkyl, alkynyl, cyclopropyl, alkylamino, dialkylamino, alkylthio, alkylsulfonyl, carboxy, alkoxycarbonyl, alkylaminosulfonyl, dialkylaminosulfonyl, —CONH₂, alkylaminocarbonyl, dialkylaminocarbonyl, 3-, 4-, or 5-membered heterocylyl, hydroxy, alkoxy, cyano, halo, haloalkyl or haloalkoxy;

R³, R⁴, and R⁵ are independently hydrogen, alkyl, hydroxy, alkoxy, halo, haloalkyl, haloalkoxy, carboxy, alkoxycarbonyl, cyano, —CONH₂, amino, or monosubstituted or disubstituted amino;

X is alkylene, -alkynylene-NR^{a}—, cycloalkylene, -alkylene-O—, -cycloalkylene-NR^{a}—, -(alkylene)-NR^{a}—, -phenylene-NR^{a}— (where each R^{a} is hydrogen, alkyl or cycloalkyl), or

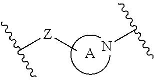

(where Z is bond or alkylene, and ring A is heterocycloamino optionally substituted with one or two substituents independently selected from alkyl, hydroxy, alkoxy, or fluoro);

Y is —CO— or —SO₂—;

R^{c} is alkyl, haloalkoxy, substituted alkyl, cycloalkyl, 1-(alkyleneR^{b})-cycloalkan-1-yl (where R^{b} is amino, alkylamino, dialkylamino, hydroxy, or monocyclic heteroaryl), 1-NR^{d}R^{e}cycloalkan-1-yl (where R^{d} and R^{e} are independently hydrogen, alkyl, or cycloalkyl), or 3 to 6 membered saturated monocyclic heterocyclyl containing one or two heteroatoms selected from N, O, or S and optionally substituted with one or two substituents independently selected from hydroxy, alkoxy, alkyl, fluoro, aminoalkyl, hydroxyalkyl, or alkoxyalkyl;

and/or a pharmaceutically acceptable salt thereof.

In one embodiment, the compounds of Formula (I) are reversible covalent inhibitors.

In another embodiment the compounds of the present disclosure form a reversible covalent bond to Cys 481 of BTK.

In a second aspect, this disclosure is directed to a pharmaceutical composition comprising a compound of Formula (I) (or any of the embodiments thereof described herein), and/or a pharmaceutically acceptable salt thereof; and a pharmaceutically acceptable excipient.

In a third aspect, this disclosure is directed to a method of treating a disease treatable by inhibition of a tyrosine kinase such as BLK, BMX, EGFR, HER2, HER4, ITK, TEC, BTK, and TXK, in particular BTK, in a patient which method comprises administering to the patient in need of such inhibition, a pharmaceutical composition comprising a compound of Formula (I) and/or a pharmaceutically acceptable salt thereof (or any of the embodiments thereof described herein), and a pharmaceutically acceptable excipient in an amount effective to achieve the treatment (therapeutic amount). In one embodiment of the third aspect, the patient is in recognized need of such treatment. In one embodiment the disease is autoimmune disease such as arthritis, inflammatory disease such as asthma, Sjogrens's disease such as dry eye, non-Sjogren's dry eye, or cancer such as B-cell proliferative disorder, e.g., diffuse large B cell lymphoma, follicular lymphoma, chronic lymphocytic lymphoma, chronic lymphocytic leukemia, B-ALL, B-cell prolymphocytic leukemia, small lymphocytic lymphoma (SLL), multiple myeloma, B-cell non-Hodgkin lymphoma, lymphoplamascytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis.

In one embodiment of this aspect, the subject in need or recognized need of such treatment is suffering from an autoimmune disease, e.g., inflammatory bowel disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, Sjogren's syndrome, multiple sclerosis, Guillain-Barre syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, or vulvodynia. In one embodiment, the disease is rheumatoid arthritis. In another embodiment the autoimmune disease is lupus. In another embodiment of this aspect, the patient in need is suffering from a heteroimmune condition or disease, e.g., graft versus host disease, transplantation, transfusion, anaphylaxis, allergy, type I hypersensitivity, allergic conjunctivitis, allergic rhinitis, or atopic dermatitis.

In another embodiment of this aspect, the patient in need or recognized need of such treatment is suffering from an inflammatory disease, e.g., asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis. In another embodiment of this aspect, the patient in need or recognized need of such treatment is suffering from inflammatory skin disease such as dermatitis, contact dermatitis, eczema, urticaria, rosacea, and scarring psoriatic lesions in the skin, joints, or other tissues or organs.

In yet another embodiment of this aspect, the patient in need or recognized need of such treatment is suffering from a cancer. In one embodiment, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplamascytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, the compound of Formula (I) and/or a pharmaceutically acceptable salt thereof (or any of the embodiments thereof described herein), is administered in combination with at least one other an anti-cancer agent e.g., the anti-cancer agent is an inhibitor of mitogen-activated protein kinase signaling, e.g., gefinitinib or imatinib, ofatumumab, bendamustine, rituaximab, U0126, PD98059, PD184352, PD0325901, ARRY-142886, SB239063, SP600125, BAY 43-9006, wortmannin, Nexavar®, Tarceva®, Sutent®, Tykerb®, Sprycel®, Crizotinib, Xalkori®, or LY294002. When combination therapy is used, the agents can be administered simultaneously or sequentially. In yet another embodiment, the patient in need or recognized need of such treatment is suffering from a thromboembolic disorder, e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In a fourth aspect, the disclosure is directed to the use of compound of Formula (I) and/or a pharmaceutical salt thereof (and any embodiments thereof described herein) as a medicament. In one embodiment, the use of compound of Formula (I) is for treating a disease mediated by a kinase, particularly BTK, wherein the disease is autoimmune disease such as arthritis, inflammatory disease such as asthma, Sjogrens's disease such as dry eye, non-Sjogren's dry eye, or cancer such as B-cell proliferative disorder, e.g., diffuse large B cell lymphoma, follicular lymphoma, chronic lymphocytic lymphoma, chronic lymphocytic leukemia, B-ALL, B-cell prolymphocytic leukemia, small lymphocytic lymphoma (SLL), multiple myeloma, B-cell non-Hodgkin lymphoma, lymphoplamascytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis.

In a fifth aspect is the use of a compound of Formula (I)) and/or a pharmaceutical salt thereof (or any of the embodiments thereof described herein), in the manufacture of a medicament for treating an inflammatory disease in a patient in need or recognized need of such treatment in which the activity of a tyrosine kinase such as BLK, BMX, EGFR, HER2, HER4, ITK, TEC, BTK, and TXK contributes to the pathology and/or symptoms of the disease. In one embodiment of this aspect, the tyrosine kinase protein is BTK. In another embodiment of this aspect, the disease is autoimmune disease such as arthritis, inflammatory disease such as asthma, Sjogrens's disease such as dry eye, non-Sjogren's dry eye, or cancer such as B-cell proliferative disorder, e.g., diffuse large B cell lymphoma, follicular lymphoma, chronic lymphocytic lymphoma, chronic lymphocytic leukemia, B-ALL, B-cell prolymphocytic leukemia, small lymphocytic lymphoma (SLL), multiple myeloma, B-cell non-Hodgkin lymphoma, lymphoplamascytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis.

In any of the aforementioned aspects involving the treatment of proliferative disorders, including cancer, are further embodiments comprising administering the compound of Formula (I)) and/or a pharmaceutical salt thereof (or any of the embodiments thereof described herein), in combination with at least one additional agent chosen from alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzamab, methotrexate, paclitaxel, Taxol™, temozolomide, thioguanine, and classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, dronabinol. When combination therapy is used, the agents can be administered simultaneously or sequentially.

In a sixth aspect, this disclosure is directed to an intermediate of Formula (II):

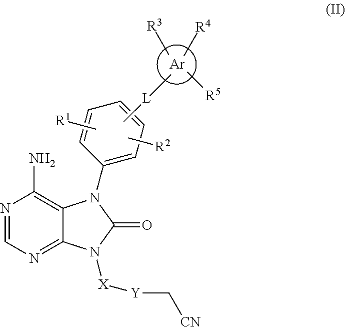

wherein:

L, Ar, R¹, R², R³, R⁴, R⁵, X and Y are as defined in compound (I) (or any of the embodiments thereof described herein)) and/or a salt thereof.

In a ninth aspect, provided is a process of preparing:

(i) a compound of Formula (I) wherein L, Ar, R¹, R², R³, R⁴, R⁵, X and Y are as defined above (or any of the embodiments thereof described herein) comprising reacting a compound of formula (II):

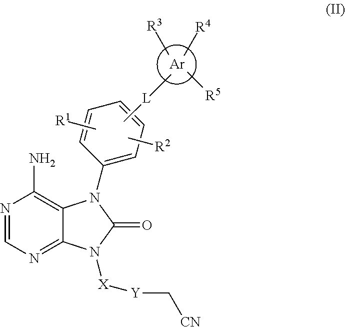

wherein:

L, Ar, R¹, R², R³, R⁴, R⁵, X and Y are as defined in compound (I) (or any of the embodiments thereof described herein), with R^{c}CHO where R^{c} is as defined in compound of Formula (I) above (or any of the embodiments thereof described herein); or

(ii) a compound of Formula (I) wherein L, Ar, R¹, R², R³, R⁴, R⁵ are as defined above (or any of the embodiments thereof described herein), X is -alkylene-O—, -cycloalkylene-NR^{a}—, -(alkylene)-NR^{a}—, -phenylene-NR^{a}— (where each R^{a} is hydrogen, alkyl or cycloalkyl), or

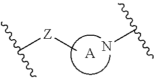

(where Z is bond or alkylene, and ring A is heterocycloamino optionally substituted with one or two substituents independently selected from alkyl, hydroxy, alkoxy, or fluoro); comprising reacting a compound of formula (III):

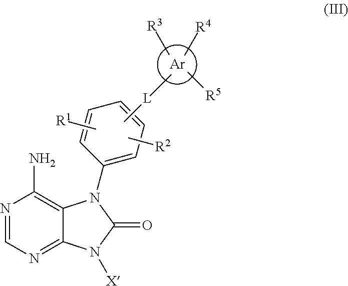

where:

L, Ar, R¹, R², R³, R⁴, and R⁵ are as defined in compound (I) (or any of the embodiments thereof described herein); and

X′ is -alkylene-OH, -cycloalkylene-NHR^{a}, -(alkylene)-NHR^{a}, -phenylene-NHR^{a} (where each R^{a} is hydrogen, alkyl or cycloalkyl), or

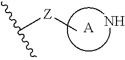

(where Z is bond or alkylene, and ring A is heterocycloamino optionally substituted with one or two substituents independently selected from alkyl, hydroxy, alkoxy, or fluoro) (or any of the embodiments thereof described herein);

with a compound of formula R^{c}HC═C(CN)COX where X is a leaving group under amido coupling reaction conditions and R^{c} is as defined in compound of Formula (I) above (or any of the embodiments thereof described herein);

(iii) optionally making an acid addition salt of a compound obtained from Step (i) or (ii) above;

(iv) optionally making a free base of a compound obtained from Step (i) or (ii) above.

### DEFINITIONS

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alkynyl” means a linear saturated monovalent hydrocarbon radical of two to six carbon atoms or a branched saturated monovalent hydrocarbon radical of three to six carbon atoms that contains a triple bond, e.g., ethynyl, propynyl, and the like.

“Alkynylene” means a linear saturated divalent hydrocarbon radical of two to six carbon atoms or a branched saturated monovalent hydrocarbon radical of three to six carbon atoms that contains a triple bond, e.g., ethynylene, propynylene, and the like.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkylthio” means a —SR radical where R is alkyl as defined above, e.g., methylthio, ethylthio, and the like.

“Alkylsulfonyl” means a —SO₂R radical where R is alkyl as defined above, e.g., methylsulfonyl, ethylsulfonyl, and the like.

“Amino” means a —NH₂.

“Alkylamino” means a —NHR radical where R is alkyl as defined above, e.g., methylamino, ethylamino, propylamino, or 2-propylamino, and the like.

“Alkoxy” means a —OR radical where R is alkyl as defined above, e.g., methoxy, ethoxy, propoxy, or 2-propoxy, n-, iso-, or tert-butoxy, and the like.

“Aminoalkyl” means a linear monovalent hydrocarbon radical of one to six carbon atoms or a branched monovalent hydrocarbon radical of three to six carbons substituted with —NR′R″ where R′ and R″ are independently hydrogen or alkyl as defined above, e.g., aminomethyl, aminoethyl, methylaminomethyl, and the like.

“Alkoxyalkyl” means a linear monovalent hydrocarbon radical of one to six carbon atoms or a branched monovalent hydrocarbon radical of three to six carbons substituted with at least one alkoxy group, (in one embodiment one or two alkoxy groups), as defined above, e.g., 2-methoxyethyl, 1-, 2-, or 3-methoxypropyl, 2-ethoxyethyl, and the like.

“Alkoxycarbonyl” means a —C(O)OR radical where R is alkyl as defined above, e.g., methoxycarbonyl, ethoxycarbonyl, and the like.

“Aminocarbonyl” means a —CONRR′ radical where R is independently hydrogen, alkyl, or substituted alkyl, each as defined herein and R′ is hydrogen, alkyl, cycloalkyl, cycloalkylalkyl, aryl, aralkyl, heteroaryl, heteroaralkyl, heterocyclyl, heterocyclylalkyl, or substituted alkyl, each as defined herein and wherein the aryl, heteroaryl, or heterocyclyl ring either alone or part of another group e.g., aralkyl, is optionally substituted with one, two, or three substituents independently selected from alkyl, alkoxy, halo, haloalkoxy, hydroxyl, carboxy, or alkoxycarbonyl, e.g., —CONH₂, methylaminocarbonyl, 2-dimethylaminocarbonyl, and the like. When R is hydrogen and R′ is alkyl in —CONRR′, the group is also referred to herein as alkylaminocarbonyl and when R and R′ are both alkyl in —CONRR′, the group is also referred to herein as dialkylaminocarbonyl.

“Aminosulfonyl” means a —SO₂NRR′ radical where R is independently hydrogen, alkyl, or substituted alkyl, each as defined herein and R′ is hydrogen, alkyl, cycloalkyl, cycloalkylalkyl, aryl, aralkyl, heteroaryl, heteroaralkyl, heterocyclyl, heterocyclylalkyl, or substituted alkyl, each as defined herein and wherein the aryl, heteroaryl, or heterocyclyl ring either alone or part of another group e.g., aralkyl, is optionally substituted with one, two, or three substituents independently selected from alkyl, alkoxy, halo, haloalkoxy, hydroxyl, carboxy, or alkoxycarbonyl, e.g., —SO₂NH₂, methylaminosulfonyl, dimethylaminosulfonyl, and the like. When R is hydrogen and R′ is alkyl in —SO₂NRR′, the group is also referred to herein as alkylaminosulfonyl and when R and R′ are both alkyl in —SO₂NRR′, the group is also referred to herein as dialkylaminosulfonyl.

“Acyl” means a —COR radical where R is alkyl, haloalkyl, cycloalkyl, cycloalkylalkyl, aryl, aralkyl, heteroaryl, heteroaralkyl, heterocyclyl, or heterocyclylalkyl, each as defined herein, and wherein the aryl, heteroaryl, or heterocyclyl ring either alone or part of another group e.g., aralkyl, is optionally substituted with one, two, or three substituents independently selected from alkyl, alkoxy, halo, haloalkoxy, hydroxyl, carboxy, or alkoxycarbonyl, e.g., acetyl, propionyl, benzoyl, pyridinylcarbonyl, and the like. When R is alkyl, the radical is also referred to herein as alkylcarbonyl.

“Aryl” means a monovalent monocyclic or bicyclic aromatic hydrocarbon radical of 6 to 10 ring atoms e.g., phenyl or naphthyl.

“Aralkyl” means a -(alkylene)-R radical where R is aryl as defined above.

“Cycloalkyl” means a cyclic saturated monovalent hydrocarbon radical of three to ten carbon atoms wherein one or two carbon atoms may be replaced by an oxo group, e.g., cyclopropyl, cyclobutyl, cyclopentyl, or cyclohexyl, and the like.

“Cycloalkylalkyl” means a -(alkylene)-R radical where R is cycloalkyl as defined above; e.g., cyclopropylmethyl, cyclobutylmethyl, cyclopentylethyl, or cyclohexylmethyl, and the like.

“Cycloalkylene” means a cyclic saturated divalent hydrocarbon radical of three to ten carbon atoms wherein one or two carbon atoms may be replaced by an oxo group, e.g., cyclopropylene, cyclobutylene, cyclopentylene, or cyclohexylene, and the like.

“1-(AlkyleneR^{b})-cycloalkan-1-yl” means a radical of following structure:

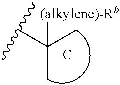

where ring C is cycloalkylene and R^{b} is amino, alkylamino, dialkylamino, hydroxy, or monocyclic heteroaryl, each term as defined herein.

“1-NR^{d}R^{e}cycloalkan-1-yl” means a radical of following structure:

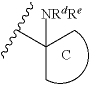

where ring C is cycloalkylene and R^{d} and R^{e} where are independently hydrogen, alkyl, or cycloalkyl, each term as defined herein.

“Carboxy” means —COOH.

“Disubstituted amino” means a —NRR′ radical where R and R′ are independently alkyl, cycloalkyl, cycloalkylalkyl, acyl, aryl, aralkyl, heteroaryl, heteroaralkyl, heterocyclyl, heterocyclylalkyl, or substituted alkyl, each as defined herein, and wherein the aryl, heteroaryl, or heterocyclyl ring either alone or part of another group e.g., aralkyl, is optionally substituted with one, two, or three substituents independently selected from alkyl, alkoxy, halo, haloalkoxy, hydroxyl, carboxy, or alkoxycarbonyl, e.g., dimethylamino, phenylmethylamino, and the like. When the R and R′ groups are alkyl, the disubstituted amino group may be referred to herein as dialkylamino.

“Halo” means fluoro, chloro, bromo, or iodo, preferably fluoro or chloro.

“Haloalkyl” means alkyl radical as defined above, which is substituted with one or more halogen atoms, (in one embodiment one to five halogen atoms. In another embodiment fluorine or chlorine), including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it is referred to in this Application as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Heterocyclyl” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C, unless stated otherwise. The heterocyclyl ring is optionally fused to a (one) aryl or heteroaryl ring as defined herein provided the aryl and heteroaryl rings are monocyclic. The heterocyclyl ring fused to monocyclic aryl or heteroaryl ring is also referred to in this Application as “bicyclic heterocyclyl” ring. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group. When the heterocyclyl group is a saturated ring and is not fused to aryl or heteroaryl ring as stated above, it is also referred to herein as saturated monocyclic heterocyclyl.

“Heterocyclylalkyl” means a -(alkylene)-R radical where R is heterocyclyl ring as defined above e.g., tetraydrofuranylmethyl, piperazinylmethyl, morpholinylethyl, and the like.

“Heterocycloamino” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C provided that at least one of the ring atoms is N. Additionally, one or two ring carbon atoms in the heterocycloamino ring can optionally be replaced by a —CO— group. When the heterocycloamino ring is unsaturated it can contain one or two ring double bonds provided that the ring is not aromatic. The heterocyloamino ring can optionally be substituted with one, two, or three substituents independently selected from alkyl, hydroxyl, alkoxy, cyano, nitro, halo, haloalkyl, haloalkoxy, alkylthio, alkylsulfonyl, carboxy, alkoxycarbonyl, aminocarbonyl or aminosulfonyl, amino, alkylamino, or dialkylamino unless otherwise stated herein.

“Heteroaryl” means a monovalent monocyclic or bicyclic aromatic radical of 5 to 10 ring atoms where one or more, (in one embodiment one, two, or three), ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

“Heteroaralkyl” means a -(alkylene)-R radical where R is heteroaryl as defined above.

“Monosubstituted amino” means a —NHR radical where R is alkyl, cycloalkyl, cycloalkylalkyl, acyl, aryl, aralkyl, heteroaryl, heteroaralkyl, heterocyclyl, heterocyclylalkyl, or substituted alkyl, each as defined herein, and wherein the aryl, heteroaryl, or heterocyclyl ring either alone or part of another group e.g., aralkyl, is optionally substituted with one, two, or three substituents independently selected from alkyl, alkoxy, halo, haloalkoxy, hydroxyl, carboxy, or alkoxycarbonyl, e.g., methylamino, phenylamino, hydroxyethylamino, and the like. When R is alkyl, the monosubstituted amino group maybe referred to herein as alkylamino.

The present disclosure also includes the prodrugs of compounds of Formula (I) (or any of the embodiments thereof described herein). The term prodrug is intended to represent covalently bonded carriers, which are capable of releasing the active ingredient of Formula (I) (or any of the embodiments thereof described herein), when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups in vivo or by routine manipulation. Prodrugs of compounds of Formula (I) (or any of the embodiments thereof described herein), include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) (or any of the embodiments thereof described herein), are also within the scope of this disclosure.

The present disclosure also includes protected derivatives of compounds of Formula (I) (or any of the embodiments thereof described herein). For example, when compounds of Formula (I) (or any of the embodiments thereof described herein), contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of Formula (I) (or any of the embodiments thereof described herein), can be prepared by methods well known in the art.

The present disclosure also includes amorphous or crystalline polymorphic forms and deuterated forms of compounds of Formula (I) (or any of the embodiments thereof described herein).

A “pharmaceutically acceptable salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, and Berge et al., Journal of Pharmaceutical Sciences, January 1977, Volume 66, Number 1, 1-19 which is incorporated herein by reference.

The compounds of the present disclosure may have asymmetric centers. Compounds of the present disclosure containing an asymmetrically substituted atom may be isolated in optically active or racemic forms. It is well known in the art how to prepare optically active forms, such as by resolution of materials. All chiral, diastereomeric, and racemic forms are within the scope of this disclosure, unless the specific stereochemistry or isomeric form is specifically indicated.

Certain compounds of Formula (I) (or any of the embodiments thereof described herein), can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth. Furthermore, when the cyclic groups such as aryl, heteroaryl, heterocyclyl are substituted, they include all the positional isomers albeit only a few examples are set forth. Furthermore, all polymorphic forms and hydrates of a compound of Formula (I) (or any of the embodiments thereof described herein), are within the scope of this disclosure.

“Oxo” or “carbonyl” means C═(O) group.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Phenylene” means a divalent phenyl group.

“Spiroheterocycloamino” means a bicyclic ring of 7 to 12 ring atoms and joined through one ring atom in which one or two ring atoms are heteroatom selected from N, O, or S(O)ₙ, where n is an integer from 0 to 2, the remaining ring atoms being C provided that at least one of the ring atoms is N. Additionally, one or two ring carbon atoms in the heterocycloamino ring can optionally be replaced by a —CO— group.

“Substituted alkyl” means alkyl group as defined herein which is substituted with one, two, or three substituents independently selected from hydroxyl, alkoxy, carboxy, cyano, carboxy, alkoxycarbonyl, alkylthio, alkylsulfonyl, halo, —CONRR′ or —NRR′ (where each R is hydrogen, alkyl, cycloalkyl, hydroxyalkyl, alkoxyalkyl, or heterocyclyl and each R′ is hydrogen, alkyl, or cycloalkyl), spiroheterocycloamino, or heterocyclyl (preferably heterocycloamino) which is optionally substituted with one or two groups independently selected from alkyl, hydroxyl, alkoxy, alkylthio, alkylsulfonyl, halo, or —CONRR′ where R and R′ are as defined above.

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease;

(2) inhibiting the disease, i.e., arresting or reducing the development of the disease or its clinical symptoms; or

(3) relieving the disease, i.e., causing regression of the disease or its clinical symptoms.

A “therapeutically effective amount” means the amount of a compound of Formula (I) and/or a pharmaceutically acceptable salt thereof (or any of the embodiments thereof described herein), that, when administered to a patient in recognized need of treatment for treating a disease, is sufficient to effect such treatment for the disease. The “therapeutically effective amount” will vary depending on the compound, the disease and its severity and the age, weight, etc., of the patient to be treated.

Representative compounds of the present disclosure are listed in the table below:

(RS), (R), or (S)-2-(2-((6-amino-8-oxo-7-(4-phenoxyphenyl)-7H-purin-9(8H)-
yl)methyl)pyrrolidine-1-carbonyl)-3-cyclopropylacrylonitrile;
(RS), (R), or (S)-2-(3-(6-amino-8-oxo-7-(4-phenoxyphenyl)-7H-purin-9(8H)-yl)piperidine-
1-carbonyl)-3-cyclopropylacrylonitrile;
2-(4-(6-amino-8-oxo-7-(4-phenoxyphenyl)-7H-purin-9(8H)-yl)piperidine-1-carbonyl)-3-
cyclopropylacrylonitrile;
(RS), (R), or (S)-N-(1-(6-amino-8-oxo-7-(4-phenoxyphenyl)-7H-purin-9(8H)-yl)propan-2-
yl)-2-cyano-3-cyclopropylacrylamide;
N-(4-(6-amino-8-oxo-7-(4-phenoxyphenyl)-7H-purin-9(8H)-yl)cyclohexyl)-2-cyano-3-
cyclopropylacrylamide;
(RS), (R), or (S)-2-(2-((6-amino-8-oxo-7-(4-phenoxyphenyl)-7H-purin-9(8H)-
yl)methyl)azetidine-1-carbonyl)-3-cyclopropylacrylonitrile;
2-(3-((6-amino-8-oxo-7-(4-phenoxyphenyl)-7H-purin-9(8H)-yl)methyl)azetidine-1-
carbonyl)-3-cyclopropylacrylonitrile;
2-(3-(6-amino-8-oxo-7-(4-phenoxyphenyl)-7H-purin-9(8H)-yl)azetidine-1-carbonyl)-3-
cyclopropylacrylonitrile;
(RS), (R), or (S)-2-(3-(6-amino-8-oxo-7-(4-phenoxyphenyl)-7H-purin-9(8H)-
yl)pyrrolidine-1-carbonyl)-3-cyclopropylacrylonitrile;
N-(3-(6-amino-8-oxo-7-(4-phenoxyphenyl)-7H-purin-9(8H)-yl)-2,2-dimethylpropyl)-2-
cyano-3-cyclopropylacrylamide;
N-(2-(6-amino-8-oxo-7-(4-phenoxyphenyl)-7H-purin-9(8H)-yl)ethyl)-2-cyano-3-
cyclopropylacrylamide;
N-(2-(6-amino-8-oxo-7-(4-phenoxyphenyl)-7H-purin-9(8H)-yl)ethyl)-2-cyano-3-
cyclopropyl-N-methylacrylamide;
(RS), (R), or (S)-2-(2-((6-amino-8-oxo-7-(4-phenoxyphenyl)-7H-purin-9(8H)-
yl)methyl)pyrrolidine-1-carbonyl)-4,4-dimethylpent-2-enenitrile;
(RS), (R), or (S)-2-(3-(6-amino-8-oxo-7-(4-phenoxyphenyl)-7H-purin-9(8H)-yl)piperidine-
1-carbonyl)-4,4-dimethylpent-2-enenitrile;
2-(4-(6-amino-8-oxo-7-(4-phenoxyphenyl)-7H-purin-9(8H)-yl)piperidine-1-carbonyl)-4,4-
dimethylpent-2-enenitrile;
(RS), (R), or (S)-N-(1-(6-amino-8-oxo-7-(4-phenoxyphenyl)-7H-purin-9(8H)-yl)propan-2-
yl)-2-cyano-4,4-dimethylpent-2-enamide;
N-(4-(6-amino-8-oxo-7-(4-phenoxyphenyl)-7H-purin-9(8H)-yl)cyclohexyl)-2-cyano-4,4-
dimethylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-4,4-dimethylpent-2-enenitrile;
2-(3-((6-amino-8-oxo-7-(4-phenoxyphenyl)-7H-purin-9(8H)-yl)methyl)azetidine-1-
carbonyl)-4,4-dimethylpent-2-enenitrile;
2-(3-(6-amino-8-oxo-7-(4-phenoxyphenyl)-7H-purin-9(8H)-yl)azetidine-1-carbonyl)-4,4-
dimethylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4,4-dimethylpent-2-enenitrile;
N-(3-(6-amino-8-oxo-7-(4-phenoxyphenyl)-7H-purin-9(8H)-yl)-2,2-dimethylpropyl)-2-
cyano-4,4-dimethylpent-2-enamide;
N-(2-(6-amino-8-oxo-7-(4-phenoxyphenyl)-7H-purin-9(8H)-yl)ethyl)-2-cyano-4,4-
dimethylpent-2-enamide;
N-(2-(6-amino-8-oxo-7-(4-phenoxyphenyl)-7H-purin-9(8H)-yl)ethyl)-2-cyano-N,4,4-
trimethylpent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-methylpent-2-enenitrile;
(RS), (R), or (S)-2-(3-(6-amino-8-oxo-7-(4-phenoxyphenyl)-7H-purin-9(8H)-yl)piperidine-
1-carbonyl)-4-methylpent-2-enenitrile;
2-(4-(6-amino-8-oxo-7-(4-phenoxyphenyl)-7H-purin-9(8H)-yl)piperidine-1-carbonyl)-4-
methylpent-2-enenitrile;
(RS), (R), or (S)-N-(1-(6-amino-8-oxo-7-(4-phenoxyphenyl)-7H-purin-9(8H)-yl)propan-2-
yl)-2-cyano-4-methylpent-2-enamide;
N-(4-(6-amino-8-oxo-7-(4-phenoxyphenyl)-7H-purin-9(8H)-yl)cyclohexyl)-2-cyano-4-
methylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-methylpent-2-enenitrile;
2-(3-((6-amino-8-oxo-7-(4-phenoxyphenyl)-7H-purin-9(8H)-yl)methyl)azetidine-1-
carbonyl)-4-methylpent-2-enenitrile;
2-(3-(6-amino-8-oxo-7-(4-phenoxyphenyl)-7H-purin-9(8H)-yl)azetidine-1-carbonyl)-4-
methylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-methylpent-2-enenitrile;
N-(3-(6-amino-8-oxo-7-(4-phenoxyphenyl)-7H-purin-9(8H)-yl)-2,2-dimethylpropyl)-2-
cyano-4-methylpent-2-enamide;
N-(2-(6-amino-8-oxo-7-(4-phenoxyphenyl)-7H-purin-9(8H)-yl)ethyl)-2-cyano-4-
methylpent-2-enamide;
N-(2-(6-amino-8-oxo-7-(4-phenoxyphenyl)-7H-purin-9(8H)-yl)ethyl)-2-cyano-N,4-
dimethylpent-2-enamide;
(RS), (R), or (S)-4-amino-2-(2-((6-amino-8-oxo-7-(4-phenoxyphenyl)-7H-purin-9(8H)-
yl)methyl)pyrrolidine-1-carbonyl)-4-methylpent-2-enenitrile;
(RS), (R), or (S)-4-amino-2-(3-(6-amino-8-oxo-7-(4-phenoxyphenyl)-7H-purin-9(8H)-
yl)piperidine-1-carbonyl)-4-methylpent-2-enenitrile;
4-amino-2-(4-(6-amino-8-oxo-7-(4-phenoxyphenyl)-7H-purin-9(8H)-yl)piperidine-1-
carbonyl)-4-methylpent-2-enenitrile;
(RS), (R), or (S)-4-amino-N-(1-(6-amino-8-oxo-7-(4-phenoxyphenyl)-7H-purin-9(8H)-
yl)propan-2-yl)-2-cyano-4-methylpent-2-enamide;
4-amino-N-(4-(6-amino-8-oxo-7-(4-phenoxyphenyl)-7H-purin-9(8H)-yl)cyclohexyl)-2-
cyano-4-methylpent-2-enamide;
(RS), (R), or (S)-4-amino-2-(2-((6-amino-8-oxo-7-(4-phenoxyphenyl)-7H-purin-9(8H)-
yl)methyl)azetidine-1-carbonyl)-4-methylpent-2-enenitrile;
4-amino-2-(3-((6-amino-8-oxo-7-(4-phenoxyphenyl)-7H-purin-9(8H)-yl)methyl)azetidine-
1-carbonyl)-4-methylpent-2-enenitrile;
4-amino-2-(3-(6-amino-8-oxo-7-(4-phenoxyphenyl)-7H-purin-9(8H)-yl)azetidine-1-
carbonyl)-4-methylpent-2-enenitrile;
(RS), (R), or (S)-4-amino-2-(3-(6-amino-8-oxo-7-(4-phenoxyphenyl)-7H-purin-9(8H)-
yl)pyrrolidine-1-carbonyl)-4-methylpent-2-enenitrile;
4-amino-N-(3-(6-amino-8-oxo-7-(4-phenoxyphenyl)-7H-purin-9(8H)-yl)-2,2-
dimethylpropyl)-2-cyano-4-methylpent-2-enamide;
4-amino-N-(2-(6-amino-8-oxo-7-(4-phenoxyphenyl)-7H-purin-9(8H)-yl)ethyl)-2-cyano-4-
methylpent-2-enamide;
4-amino-N-(2-(6-amino-8-oxo-7-(4-phenoxyphenyl)-7H-purin-9(8H)-yl)ethyl)-2-cyano-
N,4-dimethylpent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-ethoxy-4-methylpent-2-enenitrile;
(RS), (R), or (S)-2-(3-(6-amino-8-oxo-7-(4-phenoxyphenyl)-7H-purin-9(8H)-yl)piperidine-
1-carbonyl)-4-ethoxy-4-methylpent-2-enenitrile;
2-(4-(6-amino-8-oxo-7-(4-phenoxyphenyl)-7H-purin-9(8H)-yl)piperidine-1-carbonyl)-4-
ethoxy-4-methylpent-2-enenitrile;
(RS), (R), or (S)-N-(1-(6-amino-8-oxo-7-(4-phenoxyphenyl)-7H-purin-9(8H)-yl)propan-2-
yl)-2-cyano-4-ethoxy-4-methylpent-2-enamide;
N-(4-(6-amino-8-oxo-7-(4-phenoxyphenyl)-7H-purin-9(8H)-yl)cyclohexyl)-2-cyano-4-
ethoxy-4-methylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-ethoxy-4-methylpent-2-enenitrile;
2-(3-((6-amino-8-oxo-7-(4-phenoxyphenyl)-7H-purin-9(8H)-yl)methyl)azetidine-1-
carbonyl)-4-ethoxy-4-methylpent-2-enenitrile;
2-(3-(6-amino-8-oxo-7-(4-phenoxyphenyl)-7H-purin-9(8H)-yl)azetidine-1-carbonyl)-4-
ethoxy-4-methylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-ethoxy-4-methylpent-2-enenitrile;
N-(3-(6-amino-8-oxo-7-(4-phenoxyphenyl)-7H-purin-9(8H)-yl)-2,2-dimethylpropyl)-2-
cyano-4-ethoxy-4-methylpent-2-enamide;
N-(2-(6-amino-8-oxo-7-(4-phenoxyphenyl)-7H-purin-9(8H)-yl)ethyl)-2-cyano-4-ethoxy-4-
methylpent-2-enamide;
N-(2-(6-amino-8-oxo-7-(4-phenoxyphenyl)-7H-purin-9(8H)-yl)ethyl)-2-cyano-4-ethoxy-
N,4-dimethylpent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-(dimethylamino)-4-methylpent-2-enenitrile;
(RS), (R), or (S)-2-(3-(6-amino-8-oxo-7-(4-phenoxyphenyl)-7H-purin-9(8H)-yl)piperidine-
1-carbonyl)-4-(dimethylamino)-4-methylpent-2-enenitrile;
2-(4-(6-amino-8-oxo-7-(4-phenoxyphenyl)-7H-purin-9(8H)-yl)piperidine-1-carbonyl)-4-
(dimethylamino)-4-methylpent-2-enenitrile;
(RS), (R), or (S)-N-(1-(6-amino-8-oxo-7-(4-phenoxyphenyl)-7H-purin-9(8H)-yl)propan-2-
yl)-2-cyano-4-(dimethylamino)-4-methylpent-2-enamide;
N-(4-(6-amino-8-oxo-7-(4-phenoxyphenyl)-7H-purin-9(8H)-yl)cyclohexyl)-2-cyano-4-
(dimethylamino)-4-methylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-(dimethylamino)-4-methylpent-2-enenitrile;
2-(3-((6-amino-8-oxo-7-(4-phenoxyphenyl)-7H-purin-9(8H)-yl)methyl)azetidine-1-
carbonyl)-4-(dimethylamino)-4-methylpent-2-enenitrile;
2-(3-(6-amino-8-oxo-7-(4-phenoxyphenyl)-7H-purin-9(8H)-yl)azetidine-1-carbonyl)-4-
(dimethylamino)-4-methylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-(dimethylamino)-4-methylpent-2-enenitrile;
N-(3-(6-amino-8-oxo-7-(4-phenoxyphenyl)-7H-purin-9(8H)-yl)-2,2-dimethylpropyl)-2-
cyano-4-(dimethylamino)-4-methylpent-2-enamide;
N-(2-(6-amino-8-oxo-7-(4-phenoxyphenyl)-7H-purin-9(8H)-yl)ethyl)-2-cyano-4-
(dimethylamino)-4-methylpent-2-enamide;
N-(2-(6-amino-8-oxo-7-(4-phenoxyphenyl)-7H-purin-9(8H)-yl)ethyl)-2-cyano-4-
(dimethylamino)-N,4-dimethylpent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-methyl-4-morpholinopent-2-enenitrile;
(RS), (R), or (S)-2-(3-(6-amino-8-oxo-7-(4-phenoxyphenyl)-7H-purin-9(8H)-yl)piperidine-
1-carbonyl)-4-methyl-4-morpholinopent-2-enenitrile;
2-(4-(6-amino-8-oxo-7-(4-phenoxyphenyl)-7H-purin-9(8H)-yl)piperidine-1-carbonyl)-4-
methyl-4-morpholinopent-2-enenitrile;
(RS), (R), or (S)-N-(1-(6-amino-8-oxo-7-(4-phenoxyphenyl)-7H-purin-9(8H)-yl)propan-2-
yl)-2-cyano-4-methyl-4-morpholinopent-2-enamide;
N-(4-(6-amino-8-oxo-7-(4-phenoxyphenyl)-7H-purin-9(8H)-yl)cyclohexyl)-2-cyano-4-
methyl-4-morpholinopent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-methyl-4-morpholinopent-2-enenitrile;
2-(3-((6-amino-8-oxo-7-(4-phenoxyphenyl)-7H-purin-9(8H)-yl)methyl)azetidine-1-
carbonyl)-4-methyl-4-morpholinopent-2-enenitrile;
2-(3-(6-amino-8-oxo-7-(4-phenoxyphenyl)-7H-purin-9(8H)-yl)azetidine-1-carbonyl)-4-
methyl-4-morpholinopent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-methyl-4-morpholinopent-2-enenitrile;
N-(3-(6-amino-8-oxo-7-(4-phenoxyphenyl)-7H-purin-9(8H)-yl)-2,2-dimethylpropyl)-2-
cyano-4-methyl-4-morpholinopent-2-enamide;
N-(2-(6-amino-8-oxo-7-(4-phenoxyphenyl)-7H-purin-9(8H)-yl)ethyl)-2-cyano-4-methyl-4-
morpholinopent-2-enamide;
N-(2-(6-amino-8-oxo-7-(4-phenoxyphenyl)-7H-purin-9(8H)-yl)ethyl)-2-cyano-N,4-
dimethyl-4-morpholinopent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-methyl-4-(methylamino)pent-2-enenitrile;
(RS), (R), or (S)-2-(3-(6-amino-8-oxo-7-(4-phenoxyphenyl)-7H-purin-9(8H)-yl)piperidine-
1-carbonyl)-4-methyl-4-(methylamino)pent-2-enenitrile;
2-(4-(6-amino-8-oxo-7-(4-phenoxyphenyl)-7H-purin-9(8H)-yl)piperidine-1-carbonyl)-4-
methyl-4-(methylamino)pent-2-enenitrile;
(RS), (R), or (S)-N-(1-(6-amino-8-oxo-7-(4-phenoxyphenyl)-7H-purin-9(8H)-yl)propan-2-
yl)-2-cyano-4-methyl-4-(methylamino)pent-2-enamide;
N-(4-(6-amino-8-oxo-7-(4-phenoxyphenyl)-7H-purin-9(8H)-yl)cyclohexyl)-2-cyano-4-
methyl-4-(methylamino)pent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-methyl-4-(methylamino)pent-2-enenitrile;
2-(3-((6-amino-8-oxo-7-(4-phenoxyphenyl)-7H-purin-9(8H)-yl)methyl)azetidine-1-
carbonyl)-4-methyl-4-(methylamino)pent-2-enenitrile;
2-(3-(6-amino-8-oxo-7-(4-phenoxyphenyl)-7H-purin-9(8H)-yl)azetidine-1-carbonyl)-4-
methyl-4-(methylamino)pent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-methyl-4-(methylamino)pent-2-enenitrile;
N-(3-(6-amino-8-oxo-7-(4-phenoxyphenyl)-7H-purin-9(8H)-yl)-2,2-dimethylpropyl)-2-
cyano-4-methyl-4-(methylamino)pent-2-enamide;
N-(2-(6-amino-8-oxo-7-(4-phenoxyphenyl)-7H-purin-9(8H)-yl)ethyl)-2-cyano-4-methyl-4-
(methylamino)pent-2-enamide;
N-(2-(6-amino-8-oxo-7-(4-phenoxyphenyl)-7H-purin-9(8H)-yl)ethyl)-2-cyano-N,4-
dimethyl-4-(methylamino)pent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-(ethylamino)-4-methylpent-2-enenitrile;
(RS), (R), or (S)-2-(3-(6-amino-8-oxo-7-(4-phenoxyphenyl)-7H-purin-9(8H)-yl)piperidine-
1-carbonyl)-4-(ethylamino)-4-methylpent-2-enenitrile;
2-(4-(6-amino-8-oxo-7-(4-phenoxyphenyl)-7H-purin-9(8H)-yl)piperidine-1-carbonyl)-4-
(ethylamino)-4-methylpent-2-enenitrile;
(RS), (R), or (S)-N-(1-(6-amino-8-oxo-7-(4-phenoxyphenyl)-7H-purin-9(8H)-yl)propan-2-
yl)-2-cyano-4-(ethylamino)-4-methylpent-2-enamide;
N-(4-(6-amino-8-oxo-7-(4-phenoxyphenyl)-7H-purin-9(8H)-yl)cyclohexyl)-2-cyano-4-
(ethylamino)-4-methylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-(ethylamino)-4-methylpent-2-enenitrile;
2-(3-((6-amino-8-oxo-7-(4-phenoxyphenyl)-7H-purin-9(8H)-yl)methyl)azetidine-1-
carbonyl)-4-(ethylamino)-4-methylpent-2-enenitrile;
2-(3-(6-amino-8-oxo-7-(4-phenoxyphenyl)-7H-purin-9(8H)-yl)azetidine-1-carbonyl)-4-
(ethylamino)-4-methylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-(ethylamino)-4-methylpent-2-enenitrile;
N-(3-(6-amino-8-oxo-7-(4-phenoxyphenyl)-7H-purin-9(8H)-yl)-2,2-dimethylpropyl)-2-
cyano-4-(ethylamino)-4-methylpent-2-enamide;
N-(2-(6-amino-8-oxo-7-(4-phenoxyphenyl)-7H-purin-9(8H)-yl)ethyl)-2-cyano-4-
(ethylamino)-4-methylpent-2-enamide;
N-(2-(6-amino-8-oxo-7-(4-phenoxyphenyl)-7H-purin-9(8H)-yl)ethyl)-2-cyano-4-
(ethylamino)-N,4-dimethylpent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-methyl-4-(methyl(oxetan-3-yl)amino)pent-2-
enenitrile;
(RS), (R), or (S)-2-(3-(6-amino-8-oxo-7-(4-phenoxyphenyl)-7H-purin-9(8H)-yl)piperidine-
1-carbonyl)-4-methyl-4-(methyl(oxetan-3-yl)amino)pent-2-enenitrile;
2-(4-(6-amino-8-oxo-7-(4-phenoxyphenyl)-7H-purin-9(8H)-yl)piperidine-1-carbonyl)-4-
methyl-4-(methyl(oxetan-3-yl)amino)pent-2-enenitrile;
(RS), (R), or (S)-N-(1-(6-amino-8-oxo-7-(4-phenoxyphenyl)-7H-purin-9(8H)-yl)propan-2-
yl)-2-cyano-4-methyl-4-(methyl(oxetan-3-yl)amino)pent-2-enamide;
N-(4-(6-amino-8-oxo-7-(4-phenoxyphenyl)-7H-purin-9(8H)-yl)cyclohexyl)-2-cyano-4-
methyl-4-(methyl(oxetan-3-yl)amino)pent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-methyl-4-(methyl(oxetan-3-yl)amino)pent-2-enenitrile;
2-(3-((6-amino-8-oxo-7-(4-phenoxyphenyl)-7H-purin-9(8H)-yl)methyl)azetidine-1-
carbonyl)-4-methyl-4-(methyl(oxetan-3-yl)amino)pent-2-enenitrile;
2-(3-(6-amino-8-oxo-7-(4-phenoxyphenyl)-7H-purin-9(8H)-yl)azetidine-1-carbonyl)-4-
methyl-4-(methyl(oxetan-3-yl)amino)pent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-methyl-4-(methyl(oxetan-3-yl)amino)pent-2-enenitrile;
N-(3-(6-amino-8-oxo-7-(4-phenoxyphenyl)-7H-purin-9(8H)-yl)-2,2-dimethylpropyl)-2-
cyano-4-methyl-4-(methyl(oxetan-3-yl)amino)pent-2-enamide;
N-(2-(6-amino-8-oxo-7-(4-phenoxyphenyl)-7H-purin-9(8H)-yl)ethyl)-2-cyano-4-methyl-4-
(methyl(oxetan-3-yl)amino)pent-2-enamide;
N-(2-(6-amino-8-oxo-7-(4-phenoxyphenyl)-7H-purin-9(8H)-yl)ethyl)-2-cyano-N,4-
dimethyl-4-(methyl(oxetan-3-yl)amino)pent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-methyl-4-(4-methylpiperazin-1-yl)pent-2-enenitrile;
(RS), (R), or (S)-2-(3-(6-amino-8-oxo-7-(4-phenoxyphenyl)-7H-purin-9(8H)-yl)piperidine-
1-carbonyl)-4-methyl-4-(4-methylpiperazin-1-yl)pent-2-enenitrile;
2-(4-(6-amino-8-oxo-7-(4-phenoxyphenyl)-7H-purin-9(8H)-yl)piperidine-1-carbonyl)-4-
methyl-4-(4-methylpiperazin-1-yl)pent-2-enenitrile;
(RS), (R), or (S)-N-(1-(6-amino-8-oxo-7-(4-phenoxyphenyl)-7H-purin-9(8H)-yl)propan-2-
yl)-2-cyano-4-methyl-4-(4-methylpiperazin-1-yl)pent-2-enamide;
N-(4-(6-amino-8-oxo-7-(4-phenoxyphenyl)-7H-purin-9(8H)-yl)cyclohexyl)-2-cyano-4-
methyl-4-(4-methylpiperazin-1-yl)pent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-methyl-4-(4-methylpiperazin-1-yl)pent-2-enenitrile;
2-(3-((6-amino-8-oxo-7-(4-phenoxyphenyl)-7H-purin-9(8H)-yl)methyl)azetidine-1-
carbonyl)-4-methyl-4-(4-methylpiperazin-1-yl)pent-2-enenitrile;
2-(3-(6-amino-8-oxo-7-(4-phenoxyphenyl)-7H-purin-9(8H)-yl)azetidine-1-carbonyl)-4-
methyl-4-(4-methylpiperazin-1-yl)pent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-methyl-4-(4-methylpiperazin-1-yl)pent-2-enenitrile;
N-(3-(6-amino-8-oxo-7-(4-phenoxyphenyl)-7H-purin-9(8H)-yl)-2,2-dimethylpropyl)-2-
cyano-4-methyl-4-(4-methylpiperazin-1-yl)pent-2-enamide;
N-(2-(6-amino-8-oxo-7-(4-phenoxyphenyl)-7H-purin-9(8H)-yl)ethyl)-2-cyano-4-methyl-4-
(4-methylpiperazin-1-yl)pent-2-enamide;
N-(2-(6-amino-8-oxo-7-(4-phenoxyphenyl)-7H-purin-9(8H)-yl)ethyl)-2-cyano-N,4-
dimethyl-4-(4-methylpiperazin-1-yl)pent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-(ethyl(oxetan-3-yl)amino)-4-methylpent-2-enenitrile;
(RS), (R), or (S)-2-(3-(6-amino-8-oxo-7-(4-phenoxyphenyl)-7H-purin-9(8H)-yl)piperidine-
1-carbonyl)-4-(ethyl(oxetan-3-yl)amino)-4-methylpent-2-enenitrile;
2-(4-(6-amino-8-oxo-7-(4-phenoxyphenyl)-7H-purin-9(8H)-yl)piperidine-1-carbonyl)-4-
(ethyl(oxetan-3-yl)amino)-4-methylpent-2-enenitrile;
(RS), (R), or (S)-N-(1-(6-amino-8-oxo-7-(4-phenoxyphenyl)-7H-purin-9(8H)-yl)propan-2-
yl)-2-cyano-4-(ethyl(oxetan-3-yl)amino)-4-methylpent-2-enamide;
N-(4-(6-amino-8-oxo-7-(4-phenoxyphenyl)-7H-purin-9(8H)-yl)cyclohexyl)-2-cyano-4-
(ethyl(oxetan-3-yl)amino)-4-methylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-(ethyl(oxetan-3-yl)amino)-4-methylpent-2-enenitrile;
2-(3-((6-amino-8-oxo-7-(4-phenoxyphenyl)-7H-purin-9(8H)-yl)methyl)azetidine-1-
carbonyl)-4-(ethyl(oxetan-3-yl)amino)-4-methylpent-2-enenitrile;
2-(3-(6-amino-8-oxo-7-(4-phenoxyphenyl)-7H-purin-9(8H)-yl)azetidine-1-carbonyl)-4-
(ethyl(oxetan-3-yl)amino)-4-methylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-(ethyl(oxetan-3-yl)amino)-4-methylpent-2-enenitrile;
N-(3-(6-amino-8-oxo-7-(4-phenoxyphenyl)-7H-purin-9(8H)-yl)-2,2-dimethylpropyl)-2-
cyano-4-(ethyl(oxetan-3-yl)amino)-4-methylpent-2-enamide;
N-(2-(6-amino-8-oxo-7-(4-phenoxyphenyl)-7H-purin-9(8H)-yl)ethyl)-2-cyano-4-
(ethyl(oxetan-3-yl)amino)-4-methylpent-2-enamide;
N-(2-(6-amino-8-oxo-7-(4-phenoxyphenyl)-7H-purin-9(8H)-yl)ethyl)-2-cyano-4-
(ethyl(oxetan-3-yl)amino)-N,4-dimethylpent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-methyl-4-(2-oxa-6-azaspiro[3.3]heptan-6-yl)pent-2-
enenitrile;
(RS), (R), or (S)-2-(3-(6-amino-8-oxo-7-(4-phenoxyphenyl)-7H-purin-9(8H)-yl)piperidine-
1-carbonyl)-4-methyl-4-(2-oxa-6-azaspiro[3.3]heptan-6-yl)pent-2-enenitrile;
2-(4-(6-amino-8-oxo-7-(4-phenoxyphenyl)-7H-purin-9(8H)-yl)piperidine-1-carbonyl)-4-
methyl-4-(2-oxa-6-azaspiro[3.3]heptan-6-yl)pent-2-enenitrile;
(RS), (R), or (S)-N-(1-(6-amino-8-oxo-7-(4-phenoxyphenyl)-7H-purin-9(8H)-yl)propan-2-
yl)-2-cyano-4-methyl-4-(2-oxa-6-azaspiro[3.3]heptan-6-yl)pent-2-enamide;
N-(4-(6-amino-8-oxo-7-(4-phenoxyphenyl)-7H-purin-9(8H)-yl)cyclohexyl)-2-cyano-4-
methyl-4-(2-oxa-6-azaspiro[3.3]heptan-6-yl)pent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-methyl-4-(2-oxa-6-azaspiro[3.3]heptan-6-yl)pent-2-
enenitrile;
2-(3-((6-amino-8-oxo-7-(4-phenoxyphenyl)-7H-purin-9(8H)-yl)methyl)azetidine-1-
carbonyl)-4-methyl-4-(2-oxa-6-azaspiro[3.3]heptan-6-yl)pent-2-enenitrile;
2-(3-(6-amino-8-oxo-7-(4-phenoxyphenyl)-7H-purin-9(8H)-yl)azetidine-1-carbonyl)-4-
methyl-4-(2-oxa-6-azaspiro[3.3]heptan-6-yl)pent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-methyl-4-(2-oxa-6-azaspiro[3.3]heptan-6-yl)pent-2-
enenitrile;;
N-(3-(6-amino-8-oxo-7-(4-phenoxyphenyl)-7H-purin-9(8H)-yl)-2,2-dimethylpropyl)-2-
cyano-4-methyl-4-(2-oxa-6-azaspiro[3.3]heptan-6-yl)pent-2-enamide;
N-(2-(6-amino-8-oxo-7-(4-phenoxyphenyl)-7H-purin-9(8H)-yl)ethyl)-2-cyano-4-methyl-4-
(2-oxa-6-azaspiro[3.3]heptan-6-yl)pent-2-enamide;
N-(2-(6-amino-8-oxo-7-(4-phenoxyphenyl)-7H-purin-9(8H)-yl)ethyl)-2-cyano-N,4-
dimethyl-4-(2-oxa-6-azaspiro[3.3]heptan-6-yl)pent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-methyl-4-(oxetan-3-ylamino)pent-2-enenitrile;
(RS), (R), or (S)-2-(3-(6-amino-8-oxo-7-(4-phenoxyphenyl)-7H-purin-9(8H)-yl)piperidine-
1-carbonyl)-4-methyl-4-(oxetan-3-ylamino)pent-2-enenitrile;
2-(4-(6-amino-8-oxo-7-(4-phenoxyphenyl)-7H-purin-9(8H)-yl)piperidine-1-carbonyl)-4-
methyl-4-(oxetan-3-ylamino)pent-2-enenitrile;
(RS), (R), or (S)-N-(1-(6-amino-8-oxo-7-(4-phenoxyphenyl)-7H-purin-9(8H)-yl)propan-2-
yl)-2-cyano-4-methyl-4-(oxetan-3-ylamino)pent-2-enamide;
N-(4-(6-amino-8-oxo-7-(4-phenoxyphenyl)-7H-purin-9(8H)-yl)cyclohexyl)-2-cyano-4-
methyl-4-(oxetan-3-ylamino)pent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-methyl-4-(oxetan-3-ylamino)pent-2-enenitrile;
2-(3-((6-amino-8-oxo-7-(4-phenoxyphenyl)-7H-purin-9(8H)-yl)methyl)azetidine-1-
carbonyl)-4-methyl-4-(oxetan-3-ylamino)pent-2-enenitrile;
2-(3-(6-amino-8-oxo-7-(4-phenoxyphenyl)-7H-purin-9(8H)-yl)azetidine-1-carbonyl)-4-
methyl-4-(oxetan-3-ylamino)pent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-methyl-4-(oxetan-3-ylamino)pent-2-enenitrile;
N-(3-(6-amino-8-oxo-7-(4-phenoxyphenyl)-7H-purin-9(8H)-yl)-2,2-dimethylpropyl)-2-
cyano-4-methyl-4-(oxetan-3-ylamino)pent-2-enamide;
N-(2-(6-amino-8-oxo-7-(4-phenoxyphenyl)-7H-purin-9(8H)-yl)ethyl)-2-cyano-4-methyl-4-
(oxetan-3-ylamino)pent-2-enamide;
N-(2-(6-amino-8-oxo-7-(4-phenoxyphenyl)-7H-purin-9(8H)-yl)ethyl)-2-cyano-N,4-
dimethyl-4-(oxetan-3-ylamino)pent-2-enamide;
2-(2-((6-amino-8-oxo-7-(4-phenoxyphenyl)-7H-purin-9(8H)-yl)methyl)pyrrolidine-1-
carbonyl)-4-methyl-4-(piperazin-1-yl)pent-2-enenitrile;
(RS), (R), or (S)-2-(3-(6-amino-8-oxo-7-(4-phenoxyphenyl)-7H-purin-9(8H)-yl)piperidine-
1-carbonyl)-4-methyl-4-(piperazin-1-yl)pent-2-enenitrile;
2-(4-(6-amino-8-oxo-7-(4-phenoxyphenyl)-7H-purin-9(8H)-yl)piperidine-1-carbonyl)-4-
methyl-4-(piperazin-1-yl)pent-2-enenitrile;
(RS), (R), or (S)-N-(1-(6-amino-8-oxo-7-(4-phenoxyphenyl)-7H-purin-9(8H)-yl)propan-2-
yl)-2-cyano-4-methyl-4-(piperazin-1-yl)pent-2-enamide;
N-(4-(6-amino-8-oxo-7-(4-phenoxyphenyl)-7H-purin-9(8H)-yl)cyclohexyl)-2-cyano-4-
methyl-4-(piperazin-1-yl)pent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-methyl-4-(piperazin-1-yl)pent-2-enenitrile;
2-(3-((6-amino-8-oxo-7-(4-phenoxyphenyl)-7H-purin-9(8H)-yl)methyl)azetidine-1-
carbonyl)-4-methyl-4-(piperazin-1-yl)pent-2-enenitrile;
2-(3-(6-amino-8-oxo-7-(4-phenoxyphenyl)-7H-purin-9(8H)-yl)azetidine-1-carbonyl)-4-
methyl-4-(piperazin-1-yl)pent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-methyl-4-(piperazin-1-yl)pent-2-enenitrile;
N-(3-(6-amino-8-oxo-7-(4-phenoxyphenyl)-7H-purin-9(8H)-yl)-2,2-dimethylpropyl)-2-
cyano-4-methyl-4-(piperazin-1-yl)pent-2-enamide;
N-(2-(6-amino-8-oxo-7-(4-phenoxyphenyl)-7H-purin-9(8H)-yl)ethyl)-2-cyano-4-methyl-4-
(piperazin-1-yl)pent-2-enamide;
N-(2-(6-amino-8-oxo-7-(4-phenoxyphenyl)-7H-purin-9(8H)-yl)ethyl)-2-cyano-N,4-
dimethyl-4-(piperazin-1-yl)pent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-3-(3-methyloxetan-3-yl)acrylonitrile;
(RS), (R), or (S)-2-(3-(6-amino-8-oxo-7-(4-phenoxyphenyl)-7H-purin-9(8H)-yl)piperidine-
1-carbonyl)-3-(3-methyloxetan-3-yl)acrylonitrile;
2-(4-(6-amino-8-oxo-7-(4-phenoxyphenyl)-7H-purin-9(8H)-yl)piperidine-1-carbonyl)-3-
(3-methyloxetan-3-yl)acrylonitrile;
(RS), (R), or (S)-N-(1-(6-amino-8-oxo-7-(4-phenoxyphenyl)-7H-purin-9(8H)-yl)propan-2-
yl)-2-cyano-3-(3-methyloxetan-3-yl)acrylamide;
N-(4-(6-amino-8-oxo-7-(4-phenoxyphenyl)-7H-purin-9(8H)-yl)cyclohexyl)-2-cyano-3-(3-
methyloxetan-3-yl)acrylamide;
(RS), (R), or (S)-2-(2-((6-amino-8-oxo-7-(4-phenoxyphenyl)-7H-purin-9(8H)-
yl)methyl)azetidine-1-carbonyl)-3-(3-methyloxetan-3-yl)acrylonitrile;
2-(3-((6-amino-8-oxo-7-(4-phenoxyphenyl)-7H-purin-9(8H)-yl)methyl)azetidine-1-
carbonyl)-3-(3-methyloxetan-3-yl)acrylonitrile;
2-(3-(6-amino-8-oxo-7-(4-phenoxyphenyl)-7H-purin-9(8H)-yl)azetidine-1-carbonyl)-3-(3-
methyloxetan-3-yl)acrylonitrile;
(RS), (R), or (S)-2-(3-(6-amino-8-oxo-7-(4-phenoxyphenyl)-7H-purin-9(8H)-
yl)pyrrolidine-1-carbonyl)-3-(3-methyloxetan-3-yl)acrylonitrile;
N-(3-(6-amino-8-oxo-7-(4-phenoxyphenyl)-7H-purin-9(8H)-yl)-2,2-dimethylpropyl)-2-
cyano-3-(3-methyloxetan-3-yl)acrylamide;
N-(2-(6-amino-8-oxo-7-(4-phenoxyphenyl)-7H-purin-9(8H)-yl)ethyl)-2-cyano-3-(3-
methyloxetan-3-yl)acrylamide;
N-(2-(6-amino-8-oxo-7-(4-phenoxyphenyl)-7H-purin-9(8H)-yl)ethyl)-2-cyano-N-methyl-
3-(3-methyloxetan-3-yl)acrylamide;
(RS), (R), or (S)-2-(2-((6-amino-8-oxo-7-(4-phenoxyphenyl)-7H-purin-9(8H)-
yl)methyl)pyrrolidine-1-carbonyl)-4-methyl-4-(piperidin-1-yl)pent-2-enenitrile;
(RS), (R), or (S)-2-(3-(6-amino-8-oxo-7-(4-phenoxyphenyl)-7H-purin-9(8H)-yl)piperidine-
1-carbonyl)-4-methyl-4-(piperidin-1-yl)pent-2-enenitrile;
2-(4-(6-amino-8-oxo-7-(4-phenoxyphenyl)-7H-purin-9(8H)-yl)piperidine-1-carbonyl)-4-
methyl-4-(piperidin-1-yl)pent-2-enenitrile;
(RS), (R), or (S)-N-(1-(6-amino-8-oxo-7-(4-phenoxyphenyl)-7H-purin-9(8H)-yl)propan-2-
yl)-2-cyano-4-methyl-4-(piperidin-1-yl)pent-2-enamide;
N-(4-(6-amino-8-oxo-7-(4-phenoxyphenyl)-7H-purin-9(8H)-yl)cyclohexyl)-2-cyano-4-
methyl-4-(piperidin-1-yl)pent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-methyl-4-(piperidin-1-yl)pent-2-enenitrile;
2-(3-((6-amino-8-oxo-7-(4-phenoxyphenyl)-7H-purin-9(8H)-yl)methyl)azetidine-1-
carbonyl)-4-methyl-4-(piperidin-1-yl)pent-2-enenitrile;
2-(3-(6-amino-8-oxo-7-(4-phenoxyphenyl)-7H-purin-9(8H)-yl)azetidine-1-carbonyl)-4-
methyl-4-(piperidin-1-yl)pent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-methyl-4-(piperidin-1-yl)pent-2-enenitrile;
N-(3-(6-amino-8-oxo-7-(4-phenoxyphenyl)-7H-purin-9(8H)-yl)-2,2-dimethylpropyl)-2-
cyano-4-methyl-4-(piperidin-1-yl)pent-2-enamide;
N-(2-(6-amino-8-oxo-7-(4-phenoxyphenyl)-7H-purin-9(8H)-yl)ethyl)-2-cyano-4-methyl-4-
(piperidin-1-yl)pent-2-enamide;
N-(2-(6-amino-8-oxo-7-(4-phenoxyphenyl)-7H-purin-9(8H)-yl)ethyl)-2-cyano-N,4-
dimethyl-4-(piperidin-1-yl)pent-2-enamide;
(R,S), (R,R), (S,R)or (S,S)-2-(2-((6-amino-8-oxo-7-(4-phenoxyphenyl)-7H-purin-9(8H)-
yl)methyl)pyrrolidine-1-carbonyl)-3-(pyrrolidin-2-yl)acrylonitrile;
(R,S), (R,R), (S,R)or (S,S)-2-(3-(6-amino-8-oxo-7-(4-phenoxyphenyl)-7H-purin-9(8H)-
yl)piperidine-1-carbonyl)-3-(pyrrolidin-2-yl)acrylonitrile;
2-(4-(6-amino-8-oxo-7-(4-phenoxyphenyl)-7H-purin-9(8H)-yl)piperidine-1-carbonyl)-3-
(pyrrolidin-2-yl)acrylonitrile;
(R,S), (R,R), (S,R)or (S,S)-N-(1-(6-amino-8-oxo-7-(4-phenoxyphenyl)-7H-purin-9(8H)-
yl)propan-2-yl)-2-cyano-3-(pyrrolidin-2-yl)acrylamide;
(RS), (R), or (S)-N-(4-(6-amino-8-oxo-7-(4-phenoxyphenyl)-7H-purin-9(8H)-
yl)cyclohexyl)-2-cyano-3-(pyrrolidin-2-yl)acrylamide;
(R,S), (R,R), (S,R)or (S,S)-2-(2-((6-amino-8-oxo-7-(4-phenoxyphenyl)-7H-purin-9(8H)-
yl)methyl)azetidine-1-carbonyl)-3-(pyrrolidin-2-yl)acrylonitrile;
(RS), (R), or (S)-2-(3-((6-amino-8-oxo-7-(4-phenoxyphenyl)-7H-purin-9(8H)-
yl)methyl)azetidine-1-carbonyl)-3-(pyrrolidin-2-yl)acrylonitrile;
(RS), (R), or (S)-2-(3-(6-amino-8-oxo-7-(4-phenoxyphenyl)-7H-purin-9(8H)-yl)azetidine-
1-carbonyl)-3-(pyrrolidin-2-yl)acrylonitrile;
(R,S), (R,R), (S,R)or (S,S)-2-(3-(6-amino-8-oxo-7-(4-phenoxyphenyl)-7H-purin-9(8H)-
yl)pyrrolidine-1-carbonyl)-3-(pyrrolidin-2-yl)acrylonitrile;
(RS), (R), or (S)-N-(3-(6-amino-8-oxo-7-(4-phenoxyphenyl)-7H-purin-9(8H)-yl)-2,2-
dimethylpropyl)-2-cyano-3-(pyrrolidin-2-yl)acrylamide;
(RS), (R), or (S)-N-(2-(6-amino-8-oxo-7-(4-phenoxyphenyl)-7H-purin-9(8H)-yl)ethyl)-2-
cyano-3-(pyrrolidin-2-yl)acrylamide;
(RS), (R), or (S)-N-(2-(6-amino-8-oxo-7-(4-phenoxyphenyl)-7H-purin-9(8H)-yl)ethyl)-2-
cyano-N-methyl-3-(pyrrolidin-2-yl)acrylamide;
(RS), (R), or (S)-2-(2-((6-amino-8-oxo-7-(4-phenoxyphenyl)-7H-purin-9(8H)-
yl)methyl)pyrrolidine-1-carbonyl)-4-(cyclopropylamino)-4-methylpent-2-enenitrile;
(RS), (R), or (S)-2-(3-(6-amino-8-oxo-7-(4-phenoxyphenyl)-7H-purin-9(8H)-yl)piperidine-
1-carbonyl)-4-(cyclopropylamino)-4-methylpent-2-enenitrile;
2-(4-(6-amino-8-oxo-7-(4-phenoxyphenyl)-7H-purin-9(8H)-yl)piperidine-1-carbonyl)-4-
(cyclopropylamino)-4-methylpent-2-enenitrile;
(RS), (R), or (S)-N-(1-(6-amino-8-oxo-7-(4-phenoxyphenyl)-7H-purin-9(8H)-yl)propan-2-
yl)-2-cyano-4-(cyclopropylamino)-4-methylpent-2-enamide;
N-(4-(6-amino-8-oxo-7-(4-phenoxyphenyl)-7H-purin-9(8H)-yl)cyclohexyl)-2-cyano-4-
(cyclopropylamino)-4-methylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-(cyclopropylamino)-4-methylpent-2-enenitrile;
2-(3-((6-amino-8-oxo-7-(4-phenoxyphenyl)-7H-purin-9(8H)-yl)methyl)azetidine-1-
carbonyl)-4-(cyclopropylamino)-4-methylpent-2-enenitrile;
2-(3-(6-amino-8-oxo-7-(4-phenoxyphenyl)-7H-purin-9(8H)-yl)azetidine-1-carbonyl)-4-
(cyclopropylamino)-4-methylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-(cyclopropylamino)-4-methylpent-2-enenitrile;
N-(3-(6-amino-8-oxo-7-(4-phenoxyphenyl)-7H-purin-9(8H)-yl)-2,2-dimethylpropyl)-2-
cyano-4-(cyclopropylamino)-4-methylpent-2-enamide;
N-(2-(6-amino-8-oxo-7-(4-phenoxyphenyl)-7H-purin-9(8H)-yl)ethyl)-2-cyano-4-
(cyclopropylamino)-4-methylpent-2-enamide;
N-(2-(6-amino-8-oxo-7-(4-phenoxyphenyl)-7H-purin-9(8H)-yl)ethyl)-2-cyano-4-
(cyclopropylamino)-N,4-dimethylpent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-3-(1-aminocyclopropyl)acrylonitrile;
(RS), (R), or (S)-2-(3-(6-amino-8-oxo-7-(4-phenoxyphenyl)-7H-purin-9(8H)-yl)piperidine-
1-carbonyl)-3-(1-aminocyclopropyl)acrylonitrile;
2-(4-(6-amino-8-oxo-7-(4-phenoxyphenyl)-7H-purin-9(8H)-yl)piperidine-1-carbonyl)-3-
(1-aminocyclopropyl)acrylonitrile;
(RS), (R), or (S)-N-(1-(6-amino-8-oxo-7-(4-phenoxyphenyl)-7H-purin-9(8H)-yl)propan-2-
yl)-3-(1-aminocyclopropyl)-2-cyanoacrylamide;
N-(4-(6-amino-8-oxo-7-(4-phenoxyphenyl)-7H-purin-9(8H)-yl)cyclohexyl)-3-(1-
aminocyclopropyl)-2-cyanoacrylamide;
(RS), (R), or (S)-2-(2-((6-amino-8-oxo-7-(4-phenoxyphenyl)-7H-purin-9(8H)-
yl)methyl)azetidine-1-carbonyl)-3-(1-aminocyclopropyl)acrylonitrile;
2-(3-((6-amino-8-oxo-7-(4-phenoxyphenyl)-7H-purin-9(8H)-yl)methyl)azetidine-1-
carbonyl)-3-(1-aminocyclopropyl)acrylonitrile;
2-(3-(6-amino-8-oxo-7-(4-phenoxyphenyl)-7H-purin-9(8H)-yl)azetidine-1-carbonyl)-3-(1-
aminocyclopropyl)acrylonitrile;
(RS), (R), or (S)-2-(3-(6-amino-8-oxo-7-(4-phenoxyphenyl)-7H-purin-9(8H)-
yl)pyrrolidine-1-carbonyl)-3-(1-aminocyclopropyl)acrylonitrile;
N-(3-(6-amino-8-oxo-7-(4-phenoxyphenyl)-7H-purin-9(8H)-yl)-2,2-dimethylpropyl)-3-(1-
aminocyclopropyl)-2-cyanoacrylamide;
N-(2-(6-amino-8-oxo-7-(4-phenoxyphenyl)-7H-purin-9(8H)-yl)ethyl)-3-(1-
aminocyclopropyl)-2-cyanoacrylamide;
N-(2-(6-amino-8-oxo-7-(4-phenoxyphenyl)-7H-purin-9(8H)-yl)ethyl)-3-(1-
aminocyclopropyl)-2-cyano-N-methylacrylamide;
(RS), (R), or (S)-2-(2-((6-amino-8-oxo-7-(4-phenoxyphenyl)-7H-purin-9(8H)-
yl)methyl)pyrrolidine-1-carbonyl)-4-(azetidin-1-yl)-4-methylpent-2-enenitrile;
(RS), (R), or (S)-2-(3-(6-amino-8-oxo-7-(4-phenoxyphenyl)-7H-purin-9(8H)-yl)piperidine-
1-carbonyl)-4-(azetidin-1-yl)-4-methylpent-2-enenitrile;
2-(4-(6-amino-8-oxo-7-(4-phenoxyphenyl)-7H-purin-9(8H)-yl)piperidine-1-carbonyl)-4-
(azetidin-1-yl)-4-methylpent-2-enenitrile;
(RS), (R), or (S)-N-(1-(6-amino-8-oxo-7-(4-phenoxyphenyl)-7H-purin-9(8H)-yl)propan-2-
yl)-4-(azetidin-1-yl)-2-cyano-4-methylpent-2-enamide;
N-(4-(6-amino-8-oxo-7-(4-phenoxyphenyl)-7H-purin-9(8H)-yl)cyclohexyl)-4-(azetidin-1-
yl)-2-cyano-4-methylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-(azetidin-1-yl)-4-methylpent-2-enenitrile;
2-(3-((6-amino-8-oxo-7-(4-phenoxyphenyl)-7H-purin-9(8H)-yl)methyl)azetidine-1-
carbonyl)-4-(azetidin-1-yl)-4-methylpent-2-enenitrile;
2-(3-(6-amino-8-oxo-7-(4-phenoxyphenyl)-7H-purin-9(8H)-yl)azetidine-1-carbonyl)-4-
(azetidin-1-yl)-4-methylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-(azetidin-1-yl)-4-methylpent-2-enenitrile;
N-(3-(6-amino-8-oxo-7-(4-phenoxyphenyl)-7H-purin-9(8H)-yl)-2,2-dimethylpropyl)-4-
(azetidin-1-yl)-2-cyano-4-methylpent-2-enamide;
N-(2-(6-amino-8-oxo-7-(4-phenoxyphenyl)-7H-purin-9(8H)-yl)ethyl)-4-(azetidin-1-yl)-2-
cyano-4-methylpent-2-enamide;
N-(2-(6-amino-8-oxo-7-(4-phenoxyphenyl)-7H-purin-9(8H)-yl)ethyl)-4-(azetidin-1-yl)-2-
cyano-N,4-dimethylpent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-methyl-4-(pyrrolidin-1-yl)pent-2-enenitrile;
(RS), (R), or (S)-2-(3-(6-amino-8-oxo-7-(4-phenoxyphenyl)-7H-purin-9(8H)-yl)piperidine-
1-carbonyl)-4-methyl-4-(pyrrolidin-1-yl)pent-2-enenitrile;
2-(4-(6-amino-8-oxo-7-(4-phenoxyphenyl)-7H-purin-9(8H)-yl)piperidine-1-carbonyl)-4-
methyl-4-(pyrrolidin-1-yl)pent-2-enenitrile;
(RS), (R), or (S)-N-(1-(6-amino-8-oxo-7-(4-phenoxyphenyl)-7H-purin-9(8H)-yl)propan-2-
yl)-2-cyano-4-methyl-4-(pyrrolidin-1-yl)pent-2-enamide;
N-(4-(6-amino-8-oxo-7-(4-phenoxyphenyl)-7H-purin-9(8H)-yl)cyclohexyl)-2-cyano-4-
methyl-4-(pyrrolidin-1-yl)pent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-methyl-4-(pyrrolidin-1-yl)pent-2-enenitrile;
2-(3-((6-amino-8-oxo-7-(4-phenoxyphenyl)-7H-purin-9(8H)-yl)methyl)azetidine-1-
carbonyl)-4-methyl-4-(pyrrolidin-1-yl)pent-2-enenitrile;
2-(3-(6-amino-8-oxo-7-(4-phenoxyphenyl)-7H-purin-9(8H)-yl)azetidine-1-carbonyl)-4-
methyl-4-(pyrrolidin-1-yl)pent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-methyl-4-(pyrrolidin-1-yl)pent-2-enenitrile;
N-(3-(6-amino-8-oxo-7-(4-phenoxyphenyl)-7H-purin-9(8H)-yl)-2,2-dimethylpropyl)-2-
cyano-4-methyl-4-(pyrrolidin-1-yl)pent-2-enamide;
N-(2-(6-amino-8-oxo-7-(4-phenoxyphenyl)-7H-purin-9(8H)-yl)ethyl)-2-cyano-4-methyl-4-
(pyrrolidin-1-yl)pent-2-enamide;
N-(2-(6-amino-8-oxo-7-(4-phenoxyphenyl)-7H-purin-9(8H)-yl)ethyl)-2-cyano-N,4-
dimethyl-4-(pyrrolidin-1-yl)pent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-(3-hydroxyazetidin-1-yl)-4-methylpent-2-enenitrile;
(RS), (R), or (S)-2-(3-(6-amino-8-oxo-7-(4-phenoxyphenyl)-7H-purin-9(8H)-yl)piperidine-
1-carbonyl)-4-(3-hydroxyazetidin-1-yl)-4-methylpent-2-enenitrile;
2-(4-(6-amino-8-oxo-7-(4-phenoxyphenyl)-7H-purin-9(8H)-yl)piperidine-1-carbonyl)-4-
(3-hydroxyazetidin-1-yl)-4-methylpent-2-enenitrile;
(RS), (R), or (S)-N-(1-(6-amino-8-oxo-7-(4-phenoxyphenyl)-7H-purin-9(8H)-yl)propan-2-
yl)-2-cyano-4-(3-hydroxyazetidin-1-yl)-4-methylpent-2-enamide;
N-(4-(6-amino-8-oxo-7-(4-phenoxyphenyl)-7H-purin-9(8H)-yl)cyclohexyl)-2-cyano-4-(3-
hydroxyazetidin-1-yl)-4-methylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-(3-hydroxyazetidin-1-yl)-4-methylpent-2-enenitrile;
2-(3-((6-amino-8-oxo-7-(4-phenoxyphenyl)-7H-purin-9(8H)-yl)methyl)azetidine-1-
carbonyl)-4-(3-hydroxyazetidin-1-yl)-4-methylpent-2-enenitrile;
2-(3-(6-amino-8-oxo-7-(4-phenoxyphenyl)-7H-purin-9(8H)-yl)azetidine-1-carbonyl)-4-(3-
hydroxyazetidin-1-yl)-4-methylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-(3-hydroxyazetidin-1-yl)-4-methylpent-2-enenitrile;
N-(3-(6-amino-8-oxo-7-(4-phenoxyphenyl)-7H-purin-9(8H)-yl)-2,2-dimethylpropyl)-2-
cyano-4-(3-hydroxyazetidin-1-yl)-4-methylpent-2-enamide;
N-(2-(6-amino-8-oxo-7-(4-phenoxyphenyl)-7H-purin-9(8H)-yl)ethyl)-2-cyano-4-(3-
hydroxyazetidin-1-yl)-4-methylpent-2-enamide;
N-(2-(6-amino-8-oxo-7-(4-phenoxyphenyl)-7H-purin-9(8H)-yl)ethyl)-2-cyano-4-(3-
hydroxyazetidin-1-yl)-N,4-dimethylpent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-(4-hydroxypiperidin-1-yl)-4-methylpent-2-enenitrile;
(RS), (R), or (S)-2-(3-(6-amino-8-oxo-7-(4-phenoxyphenyl)-7H-purin-9(8H)-yl)piperidine-
1-carbonyl)-4-(4-hydroxypiperidin-1-yl)-4-methylpent-2-enenitrile;
2-(4-(6-amino-8-oxo-7-(4-phenoxyphenyl)-7H-purin-9(8H)-yl)piperidine-1-carbonyl)-4-
(4-hydroxypiperidin-1-yl)-4-methylpent-2-enenitrile;
(RS), (R), or (S)-N-(1-(6-amino-8-oxo-7-(4-phenoxyphenyl)-7H-purin-9(8H)-yl)propan-2-
yl)-2-cyano-4-(4-hydroxypiperidin-1-yl)-4-methylpent-2-enamide;
N-(4-(6-amino-8-oxo-7-(4-phenoxyphenyl)-7H-purin-9(8H)-yl)cyclohexyl)-2-cyano-4-(4-
hydroxypiperidin-1-yl)-4-methylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-(4-hydroxypiperidin-1-yl)-4-methylpent-2-enenitrile;
2-(3-((6-amino-8-oxo-7-(4-phenoxyphenyl)-7H-purin-9(8H)-yl)methyl)azetidine-1-
carbonyl)-4-(4-hydroxypiperidin-1-yl)-4-methylpent-2-enenitrile;
2-(3-(6-amino-8-oxo-7-(4-phenoxyphenyl)-7H-purin-9(8H)-yl)azetidine-1-carbonyl)-4-(4-
hydroxypiperidin-1-yl)-4-methylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-(4-hydroxypiperidin-1-yl)-4-methylpent-2-enenitrile;
N-(3-(6-amino-8-oxo-7-(4-phenoxyphenyl)-7H-purin-9(8H)-yl)-2,2-dimethylpropyl)-2-
cyano-4-(4-hydroxypiperidin-1-yl)-4-methylpent-2-enamide;
N-(2-(6-amino-8-oxo-7-(4-phenoxyphenyl)-7H-purin-9(8H)-yl)ethyl)-2-cyano-4-(4-
hydroxypiperidin-1-yl)-4-methylpent-2-enamide;
N-(2-(6-amino-8-oxo-7-(4-phenoxyphenyl)-7H-purin-9(8H)-yl)ethyl)-2-cyano-4-(4-
hydroxypiperidin-1-yl)-N,4-dimethylpent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-5-hydroxy-4,4-dimethylpent-2-enenitrile;
(RS), (R), or (S)-2-(3-(6-amino-8-oxo-7-(4-phenoxyphenyl)-7H-purin-9(8H)-yl)piperidine-
1-carbonyl)-5-hydroxy-4,4-dimethylpent-2-enenitrile;
2-(4-(6-amino-8-oxo-7-(4-phenoxyphenyl)-7H-purin-9(8H)-yl)piperidine-1-carbonyl)-5-
hydroxy-4,4-dimethylpent-2-enenitrile;
(RS), (R), or (S)-N-(1-(6-amino-8-oxo-7-(4-phenoxyphenyl)-7H-purin-9(8H)-yl)propan-2-
yl)-2-cyano-5-hydroxy-4,4-dimethylpent-2-enamide;
N-(4-(6-amino-8-oxo-7-(4-phenoxyphenyl)-7H-purin-9(8H)-yl)cyclohexyl)-2-cyano-5-
hydroxy-4,4-dimethylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-5-hydroxy-4,4-dimethylpent-2-enenitrile;
2-(3-((6-amino-8-oxo-7-(4-phenoxyphenyl)-7H-purin-9(8H)-yl)methyl)azetidine-1-
carbonyl)-5-hydroxy-4,4-dimethylpent-2-enenitrile;
2-(3-(6-amino-8-oxo-7-(4-phenoxyphenyl)-7H-purin-9(8H)-yl)azetidine-1-carbonyl)-5-
hydroxy-4,4-dimethylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-5-hydroxy-4,4-dimethylpent-2-enenitrile;
N-(3-(6-amino-8-oxo-7-(4-phenoxyphenyl)-7H-purin-9(8H)-yl)-2,2-dimethylpropyl)-2-
cyano-5-hydroxy-4,4-dimethylpent-2-enamide;
N-(2-(6-amino-8-oxo-7-(4-phenoxyphenyl)-7H-purin-9(8H)-yl)ethyl)-2-cyano-5-hydroxy-
4,4-dimethylpent-2-enamide;
N-(2-(6-amino-8-oxo-7-(4-phenoxyphenyl)-7H-purin-9(8H)-yl)ethyl)-2-cyano-5-hydroxy-
4,4-trimethylpent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-(isopropylamino)-4-methylpent-2-enenitrile;
(RS), (R), or (S)-2-(3-(6-amino-8-oxo-7-(4-phenoxyphenyl)-7H-purin-9(8H)-yl)piperidine-
1-carbonyl)-4-(isopropylamino)-4-methylpent-2-enenitrile;
2-(4-(6-amino-8-oxo-7-(4-phenoxyphenyl)-7H-purin-9(8H)-yl)piperidine-1-carbonyl)-4-
(isopropylamino)-4-methylpent-2-enenitrile;
(RS), (R), or (S)-N-(1-(6-amino-8-oxo-7-(4-phenoxyphenyl)-7H-purin-9(8H)-yl)propan-2-
yl)-2-cyano-4-(isopropylamino)-4-methylpent-2-enamide;
N-(4-(6-amino-8-oxo-7-(4-phenoxyphenyl)-7H-purin-9(8H)-yl)cyclohexyl)-2-cyano-4-
(isopropylamino)-4-methylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-(isopropylamino)-4-methylpent-2-enenitrile;
2-(3-((6-amino-8-oxo-7-(4-phenoxyphenyl)-7H-purin-9(8H)-yl)methyl)azetidine-1-
carbonyl)-4-(isopropylamino)-4-methylpent-2-enenitrile;
2-(3-(6-amino-8-oxo-7-(4-phenoxyphenyl)-7H-purin-9(8H)-yl)azetidine-1-carbonyl)-4-
(isopropylamino)-4-methylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-(isopropylamino)-4-methylpent-2-enenitrile;
N-(3-(6-amino-8-oxo-7-(4-phenoxyphenyl)-7H-purin-9(8H)-yl)-2,2-dimethylpropyl)-2-
cyano-4-(isopropylamino)-4-methylpent-2-enamide;
N-(2-(6-amino-8-oxo-7-(4-phenoxyphenyl)-7H-purin-9(8H)-yl)ethyl)-2-cyano-4-
(isopropylamino)-4-methylpent-2-enamide;
N-(2-(6-amino-8-oxo-7-(4-phenoxyphenyl)-7H-purin-9(8H)-yl)ethyl)-2-cyano-4-
(isopropylamino)-N,4-dimethylpent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-(4-ethylpiperazin-1-yl)-4-methylpent-2-enenitrile;
(RS), (R), or (S)-2-(3-(6-amino-8-oxo-7-(4-phenoxyphenyl)-7H-purin-9(8H)-yl)piperidine-
1-carbonyl)-4-(4-ethylpiperazin-1-yl)-4-methylpent-2-enenitrile;
2-(4-(6-amino-8-oxo-7-(4-phenoxyphenyl)-7H-purin-9(8H)-yl)piperidine-1-carbonyl)-4-
(4-ethylpiperazin-1-yl)-4-methylpent-2-enenitrile;
(RS), (R), or (S)-N-(1-(6-amino-8-oxo-7-(4-phenoxyphenyl)-7H-purin-9(8H)-yl)propan-2-
yl)-2-cyano-4-(4-ethylpiperazin-1-yl)-4-methylpent-2-enamide;
N-(4-(6-amino-8-oxo-7-(4-phenoxyphenyl)-7H-purin-9(8H)-yl)cyclohexyl)-2-cyano-4-(4-
ethylpiperazin-1-yl)-4-methylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-(4-ethylpiperazin-1-yl)-4-methylpent-2-enenitrile;
2-(3-((6-amino-8-oxo-7-(4-phenoxyphenyl)-7H-purin-9(8H)-yl)methyl)azetidine-1-
carbonyl)-4-(4-ethylpiperazin-1-yl)-4-methylpent-2-enenitrile;
2-(3-(6-amino-8-oxo-7-(4-phenoxyphenyl)-7H-purin-9(8H)-yl)azetidine-1-carbonyl)-4-(4-
ethylpiperazin-1-yl)-4-methylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-(4-ethylpiperazin-1-yl)-4-methylpent-2-enenitrile;
N-(3-(6-amino-8-oxo-7-(4-phenoxyphenyl)-7H-purin-9(8H)-yl)-2,2-dimethylpropyl)-2-
cyano-4-(4-ethylpiperazin-1-yl)-4-methylpent-2-enamide;
N-(2-(6-amino-8-oxo-7-(4-phenoxyphenyl)-7H-purin-9(8H)-yl)ethyl)-2-cyano-4-(4-
ethylpiperazin-1-yl)-4-methylpent-2-enamide;
N-(2-(6-amino-8-oxo-7-(4-phenoxyphenyl)-7H-purin-9(8H)-yl)ethyl)-2-cyano-4-(4-
ethylpiperazin-1-yl)-N,4-dimethylpent-2-enamide;
(RS), (R), or (S)-2-(2-((6-amino-8-oxo-7-(4-phenoxyphenyl)-7H-purin-9(8H)-
yl)methyl)pyrrolidine-1-carbonyl)-4-((2-methoxyethyl)amino)-4-methylpent-2-enenitrile;
(RS), (R), or (S)-2-(3-(6-amino-8-oxo-7-(4-phenoxyphenyl)-7H-purin-9(8H)-yl)piperidine-
1-carbonyl)-4-((2-methoxyethyl)amino)-4-methylpent-2-enenitrile;
2-(4-(6-amino-8-oxo-7-(4-phenoxyphenyl)-7H-purin-9(8H)-yl)piperidine-1-carbonyl)-4-
((2-methoxyethyl)amino)-4-methylpent-2-enenitrile;
(RS), (R), or (S)-N-(1-(6-amino-8-oxo-7-(4-phenoxyphenyl)-7H-purin-9(8H)-yl)propan-2-
yl)-2-cyano-4-((2-methoxyethyl)amino)-4-methylpent-2-enamide;
N-(4-(6-amino-8-oxo-7-(4-phenoxyphenyl)-7H-purin-9(8H)-yl)cyclohexyl)-2-cyano-4-((2-
methoxyethyl)amino)-4-methylpent-2-enamide;
(RS), (R), or (S)-2-(2-((6-amino-8-oxo-7-(4-phenoxyphenyl)-7H-purin-9(8H)-
yl)methyl)azetidine-1-carbonyl)-4-((2-methoxyethyl)amino)-4-methylpent-2-enenitrile;
2-(3-((6-amino-8-oxo-7-(4-phenoxyphenyl)-7H-purin-9(8H)-yl)methyl)azetidine-1-
carbonyl)-4-((2-methoxyethyl)amino)-4-methylpent-2-enenitrile;
2-(3-(6-amino-8-oxo-7-(4-phenoxyphenyl)-7H-purin-9(8H)-yl)azetidine-1-carbonyl)-4-((2-
methoxyethyl)amino)-4-methylpent-2-enenitrile;
(RS), (R), or (S)-2-(3-(6-amino-8-oxo-7-(4-phenoxyphenyl)-7H-purin-9(8H)-
yl)pyrrolidine-1-carbonyl)-4-((2-methoxyethyl)amino)-4-methylpent-2-enenitrile;
N-(3-(6-amino-8-oxo-7-(4-phenoxyphenyl)-7H-purin-9(8H)-yl)-2,2-dimethylpropyl)-2-
cyano-4-((2-methoxyethyl)amino)-4-methylpent-2-enamide;
N-(2-(6-amino-8-oxo-7-(4-phenoxyphenyl)-7H-purin-9(8H)-yl)ethyl)-2-cyano-4-((2-
methoxyethyl)amino)-4-methylpent-2-enamide; or
N-(2-(6-amino-8-oxo-7-(4-phenoxyphenyl)-7H-purin-9(8H)-yl)ethyl)-2-cyano-4-((2-
methoxyethyl)amino)-N,4-dimethylpent-2-enamide;

individual (E) or (Z) isomer thereof;
or a pharmaceutically acceptable salt thereof.

### EMBODIMENTS

### Embodiment I

In one embodiment, the compound of Formula (I) or a salt thereof as defined in the Summary has the Formula (IA):

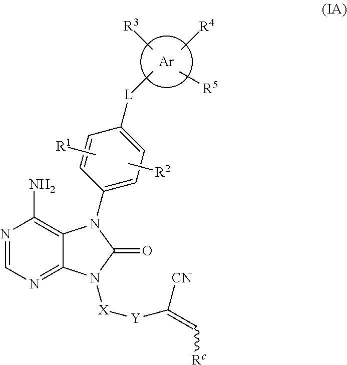

### Embodiment II

In another embodiment, the compound of Formula (I) or a salt thereof as defined in the Summary has the Formula (IB):

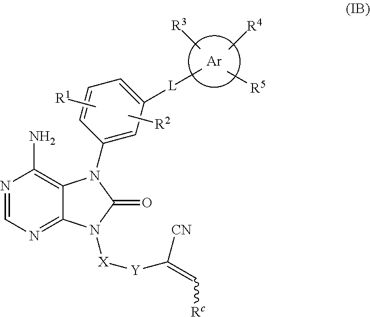

### Embodiment A

In one embodiment, the compound of Formula (I) or a salt thereof as defined in the Summary and embodiment (I) is where:

L is O; R¹ and R² are independently hydrogen, alkyl, halo, haloalkyl, or alkoxy.
(i) Within embodiment A, in one group of compounds, R¹ is hydrogen or halo (such as fluoro) and R² is hydrogen.
(ii) Within embodiment A, in another group of compounds, R¹ and R² are hydrogen.

### Embodiment B

In one embodiment, the compound of Formula (I) or a salt thereof as defined in the Summary and embodiment II is where:

L is NHCO; R¹ and R² are independently hydrogen, alkyl, halo, haloalkyl, or alkoxy.
(i) Within embodiment A, in one group of compounds, R¹ is hydrogen or halo (such as fluoro) and R² is hydrogen.
(ii) Within embodiment A, in another group of compounds, R¹ and R² are hydrogen.

### Embodiment C

In another embodiment, the compound of Formula (I) or a salt thereof as defined in the Summary, Embodiments I, II, A, and B, and groups contained therein, is where

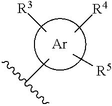

is a ring of formula:

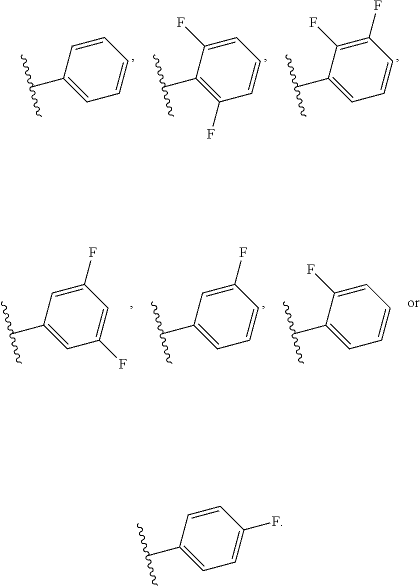

Within the groups in this embodiment in one group of compounds,

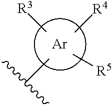

is a ring of formula: phenyl or

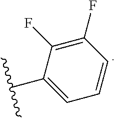 [SMILES: *c1cccc(F)c1F]

Within the groups in this embodiment, in another group of compounds

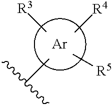

is phenyl.

### Embodiment D

In another embodiment, the compound of Formula (I) or a salt thereof as defined in the Summary, Embodiments I, II, A, B, C, and groups contained therein, is where —X— is -cycloalkylene-NR^{a}—, -(alkynylene)-NR^{a}—, -(alkylene)-NR^{a}—, -phenylene-NR^{a}— (where each R^{a} is hydrogen, alkyl or cycloalkyl), or

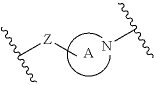

(where Z is bond or alkylene, and ring A is heterocycloamino optionally substituted with one or two substituents independently selected from alkyl, hydroxy, alkoxy, or fluoro). Within these groups of compounds in one group of compounds Y is —CO—.

Within groups in embodiment D, in group of compounds —X—Y— is:

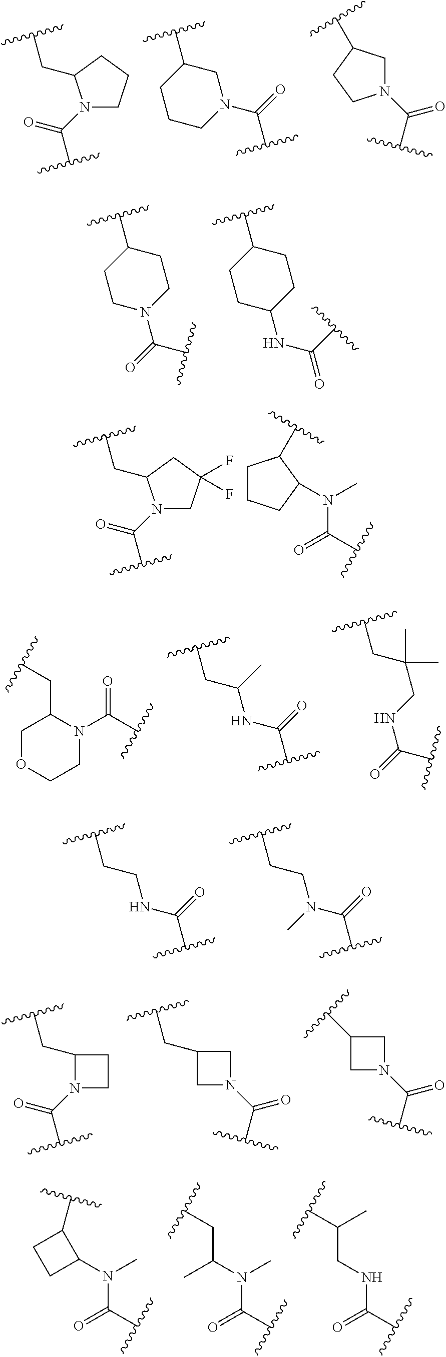

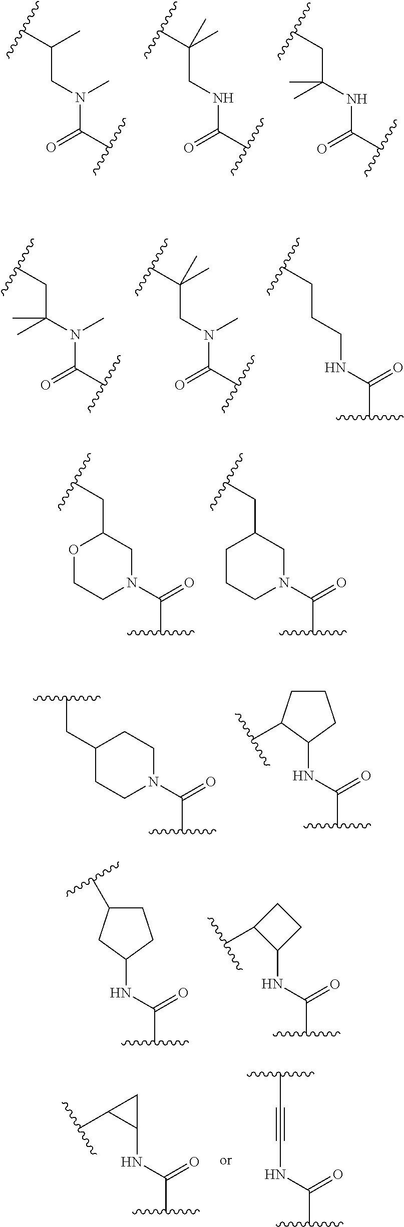

Within the groups in embodiment D, in another group of compounds —X—Y— is:

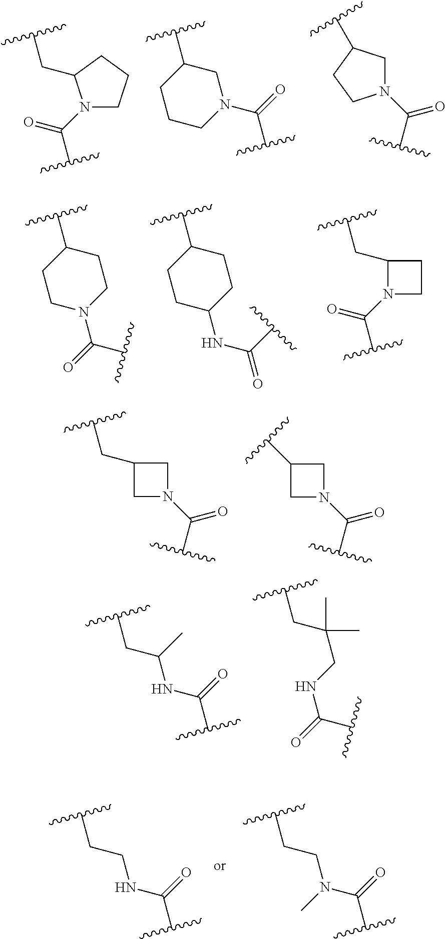

Within the groups in embodiment D, in another group of compounds —X—Y— is:

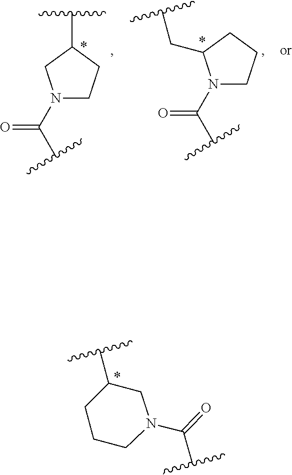 [SMILES: *C(=O)N1CCC(*)C1 ; *CC1CCCN1C(*)=O ; *C(=O)N1CCCC(*)C1]

where the stereochemistry at *C is R or S.

Within the groups in embodiment D, in yet another group of compounds, —X—Y— is:

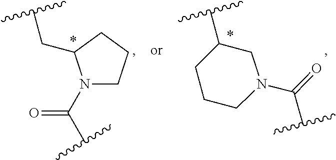 [SMILES: *CC1CCCN1C(*)=O ; *C(=O)N1CCCC(*)C1]

where the stereochemistry at *C is R or S.

### Embodiment E

In another embodiment, the compound of Formula (I) or a salt thereof as defined in the Summary, Embodiments I, II, A, B, C, D, and groups contained therein, is where R^{c} is alkyl, substituted alkyl, cycloalkyl, 1-(alkyleneR^{b})-cycloalkan-1-yl (where R^{b} is amino, alkylamino, dialkylamino, hydroxy, or monocyclic heteroaryl), 1-NR^{d}R^{e}cycloalkan-1-yl (where R^{d} and R^{e} are independently hydrogen, alkyl, or cycloalkyl), or 3 to 6 membered saturated monocyclic heterocyclyl containing one or two heteroatoms selected from N, O, or S and optionally substituted with one or two substituents independently selected from hydroxy, alkoxy, alkyl, fluoro, aminoalkyl, hydroxyalkyl, or alkoxyalkyl.

(a) Within groups of compounds in embodiment (E) and groups contained therein, in one group of compounds R^{c} is cycloalkyl. In one embodiment R^{c} is cyclopropyl.

(b) Within groups of compounds in embodiment (E) and groups contained therein, in another group of compounds R^{c} is alkyl. In one embodiment R^{c} is isopropyl or tert-butyl, more preferably isopropyl.

(c) Within groups of compounds in embodiment (E) and groups contained therein, in another group of compounds R^{c} is substituted alkyl. In one embodiment R^{c} is alkyl substituted with hydroxyl, alkoxy, —NRR′ (where R is hydrogen, alkyl, alkoxyalkyl, heterocyclyl or cycloalkyl and R′ is hydrogen or alkyl), spiroheterocycloamino, or heterocyclyl which is optionally substituted with one or two groups independently selected from alkyl. In another embodiment R^{c} is —C(CH₃)₂NH₂, —C(CH₃)₂NHCH₃, —C(CH₃)₂N(CH₃)₂, —C(CH₃)₂NHCH₂CH₃, —C(CH₃)₂NHCH(CH₃)₂, —C(CH₃)₂NHcyclopropyl, —C(CH₃)₂NH(CH₂)₂OCH₃, —C(CH₃)₂NHoxetan-3-yl, —C(CH₃)₂N(CH₃)oxetan-3-yl, —C(CH₃)₂N(CH₂CH₃)oxetan-3-yl, —C(CH₃)₂OCH₂CH₃, —C(CH₃)₂CH₂OH, —C(CH₃)₂morpholine-4-yl, —C(CH₃)₂pyrrolidin-1-yl, —C(CH₃)₂piperazin-1-yl, —C(CH₃)₂piperidin-1-yl, —C(CH₃)₂(4-hydroxypiperidin-1-yl), —C(CH₃)₂(4-methylpiperazin-1-yl), —C(CH₃)₂(4-ethylpiperazin-1-yl), —C(CH₃)₂(azetidin-1-yl), —C(CH₃)₂(3-hydroxyazetidin-1-yl), or

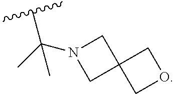 [SMILES: *C(C)(C)N1CC2(COC2)C1]

Within groups in (d), in one group of compounds R^{c} is —C(CH₃)₂NH₂, —C(CH₃)₂NHCH₃, —C(CH₃)₂N(CH₃)₂, —C(CH₃)₂NHCH₂CH₃, —C(CH₃)₂NHCH(CH₃)₂ or —C(CH₃)₂NH(CH₂)₂OCH₃. Within groups in (d), in another group of compounds R^{c} is —C(CH₃)₂NHcyclopropyl. Within groups in (d), in yet another group of compounds R^{c} is —C(CH₃)₂OCH₂CH₃. Within groups in (d), in yet another group of compounds R^{c} is —C(CH₃)₂morpholine-4-yl. Within groups in (d), in yet another group of compounds R^{c} is —C(CH₃)₂NH₂. Within groups in (d), in yet another group of compounds R^{c} is —C(CH₃)₂NHoxetan-3-yl, —C(CH₃)₂N(CH₃)oxetan-3-yl, —C(CH₃)₂N(CH₂CH₃)oxetan-3-yl.

(d) Within groups of compounds in embodiment (E) and groups contained therein, in another group of compounds R^{c} is 3 to 6 membered saturated monocyclic heterocyclyl containing one or two heteroatoms selected from N, O, or S and optionally substituted with one or two substituents selected from hydroxy, alkyl or fluoro. In one embodiment R^{c} is oxetanyl, pyrrolidinyl, piperidinyl, tetrahydrofuranyl, or tetrahydropyranyl optionally substituted with one or two substituents selected from hydroxy, alkyl or fluoro. In another embodiment R^{c} is azetidin-3-yl, 3-methylazetidin-3-yl, 3-ethylazetidin-3-yl, 3-methyloxetan-3-yl 3-ethyloxetab-3-yl, 2-pyrrolidinyl, 3-methylpyrrolidin-3-yl, 1,3-dimethylpyrrolidin-3-yl, 3- or 4-piperidinyl, 1-methylpiperidin-4-yl, 1-methylpiperidin-3-yl, 4-methylpiperidin-4-yl, 4-ethylpiperidin-4-yl, 1,4-dimethylpiperidin-4-yl, 3-methyltetrahydrofuran-3-yl, 3-ethyltetrahydrofuran-3-yl, 4-tetrahydropyran-4-yl, 4-methyltetrahydropyran-4yl, or 4-ethyltetrahydropyran-4yl.

### Embodiment F

In another embodiment, the compound of Formula (I) or salt thereof as defined in the Summary, Embodiments I, II, A, B, C, D, and groups contained therein, is where R^{c} is:

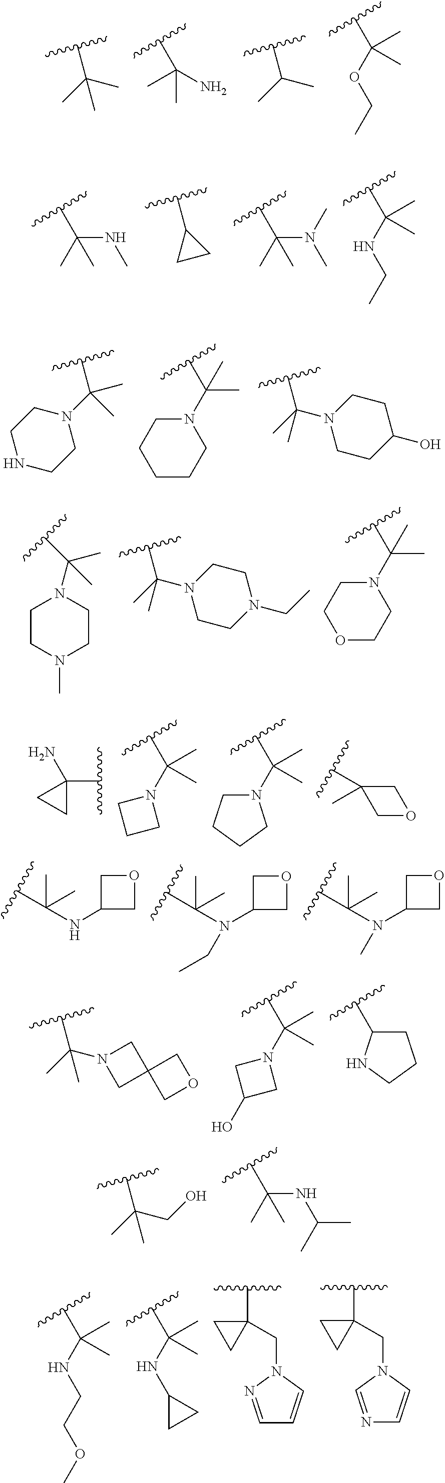

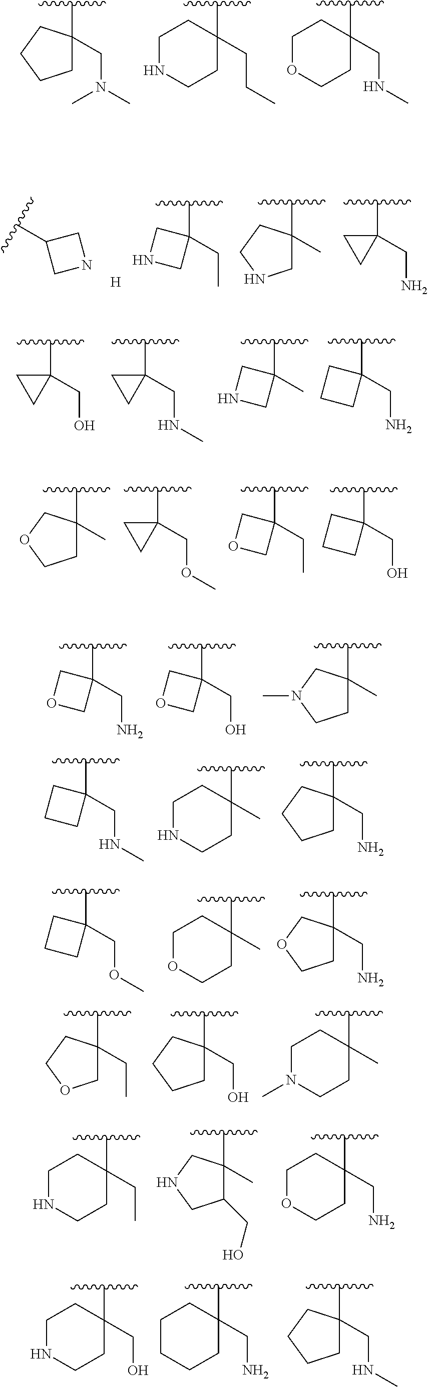

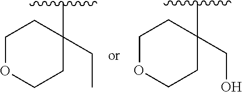

### Embodiment G

In another embodiment, the compound of formula (I) or salt thereof as defined in the Summary, Embodiments I, II, A, B, C, D, and groups contained therein, is where R^{c} is

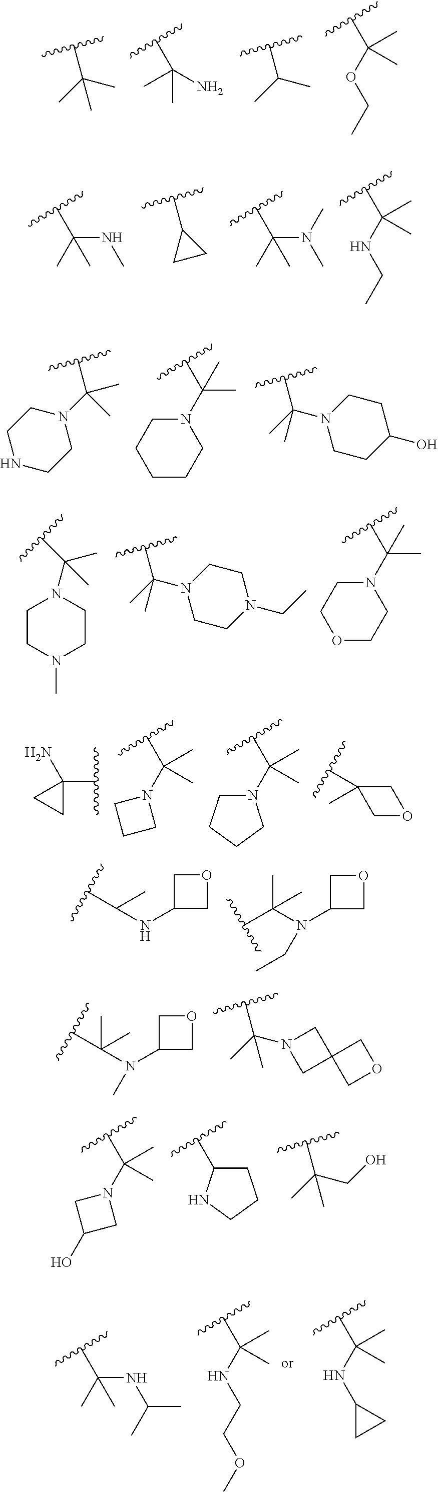

### General Synthetic Scheme

Compounds of this disclosure can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., or from about 0° C. to about 125° C. or at about room (or ambient) temperature, e.g., about 20° C.

Compounds of Formula (I) where X is a ring of formula

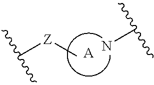

and other groups are as defined in the Summary can be prepared as illustrated and described in Scheme 1 below.

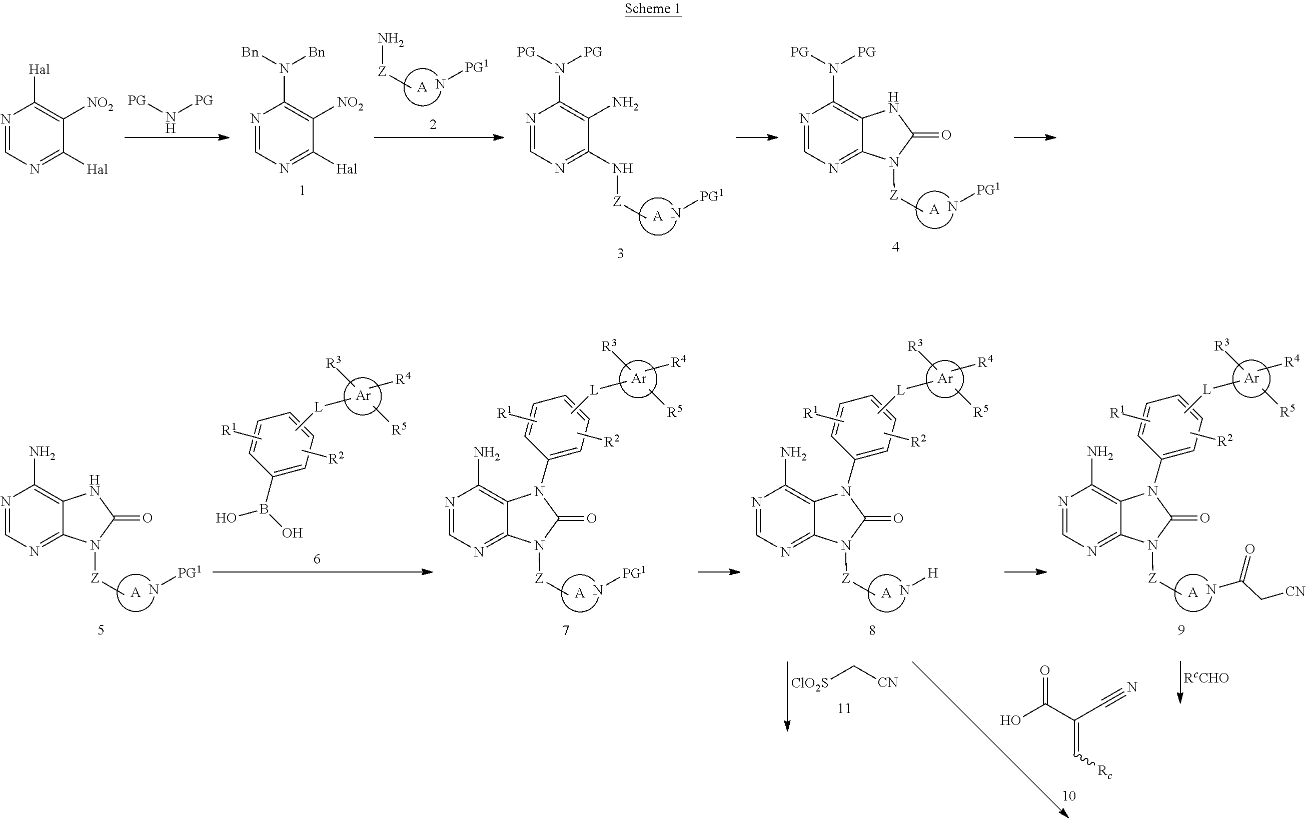

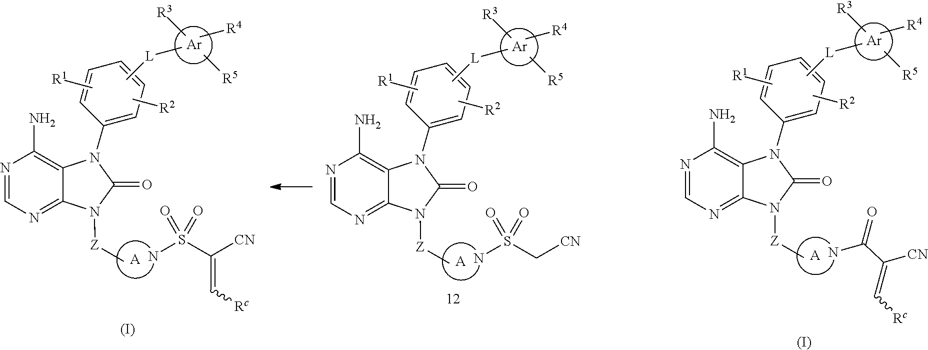

Reaction of a dihalopyrimidine such as 4,4-dichloro-5-nitropyrimidine with an amine of formula NH(PG)₂ where PG is a suitable amino protecting group such as benzyl provides a compound of formula 1. The reaction is carried out in a suitable organic solvent such as dichloromethane, and the like. Displacement of the second halo group by an amino compound of formula 2 where ring A and Z are as defined in the Summary and PG¹ is a suitable amino protecting group such as Boc, yields a compound of formula 3. The reaction is carried out in dichloromethane, dioxane, tetrahydrofuran, and the like. Compounds of formula 2 such as (R)-tert-butyl 3-aminopiperidine-1-carboxylate, (S)-tert-butyl 3-aminopiperidine-1-carboxylate, (R)-tert-butyl 3-(aminomethyl)pyrrolidine-1-carboxylate, (S)-tert-butyl 3-(aminomethyl)pyrrolidine-1-carboxylate, (R)-tert-butyl 2-(aminomethyl)azetidine-1-carboxylate, and (S)-tert-butyl 2-(aminomethyl)azetidine-1-carboxylate, are commercially available or can be prepared by methods well known in the art. Compounds of formula 3 can be cyclized to the benzimidazolones of formula 4 by heating 3 in an organic solvent such as dichloroethane and the like, with carbonyl diimidazole, phosgene or a phosgene equivalent (e.g., diphosgene or triphosgene), in the presence of a base such as triethyl amine, diisopropylethyl amine, and the like. Removal of the amino protecting group PG provides compound of formula 5. The reaction conditions utilized are based on the nature of the amino protecting group. For example, when PG is benzyl groups it can be removed via hydrogenation using a Pd/C catalyst and the like to afford a compound of formula 5. Reaction of 5 with phenylboronic acid of formula 6 where R¹, R², R³, R⁴, R⁵, and L are as defined in the Summary via a copper mediated coupling (Chan-Lam coupling) affords a compound of formula 7. Compounds of formula 6, e.g (4-phenoxyphenyl)boronic acid, 2-[4-(3-fluorophenoxy)-phenyl]-4,4,5,5-tetramethyl-1,3,2-dioxaborolane, 4-(4-fluorophenoxy)phenylboronic acid, 4-(3-fluorophenoxy)phenylboronic acid, 4-(3,5-difluorophenoxy)phenylboronic acid, 4-(4-chloro-2-fluorophenoxy)phenylboronic acid, and 4-(3-(trifluoromethyl)phenoxy)phenylboronic acid are either commercially available or can be prepared from the phenyl halide by lithium halogen exchange and quenching with triisopropyl borate.

Deprotection of the amino group in compound 7 provides compound of formula 8. The reaction conditions utilized are based on the nature of the amino protecting group. For example, when PG¹ is Boc it can be removed under acid hydrolysis reaction condition such as treatment with an acid such TFA, HCl, or the like. Compound 8 can be then converted to a compound of Formula (I) by methods well known in the art.

For example, compounds of Formula (I) where Y is —CO— can be prepared by first condensing compound 8 with 2-cyanoacetic acid under standard amide coupling conditions such as carbon diimidazole (CDI) and the like, or an acid derivative thereof provides a compound of formula 9. Condensation of a compound of formula 9 with an aldehyde of formula R^{c}CHO where R^{c} is as defined in the Summary under standard condensation reaction conditions such as using a base such as piperidine and the like, in the presence or absence of acetic acid and the like, in solvents such as ethanol and the like at temperatures ranging from room temperature to reflux then provides a compound of Formula (I). Compounds of formula R^{c}CHO are commercially available or they can be prepared by methods well known in the art eg. such as, e.g., acetaldehyde, cyclopropylaldehyde, isobutyraldehyde, 3-methyloxetane-3-carbaldehyde, 2-(dimethylamino)-2-methylpropanal, 2-methyl-2-(1-piperidyl)propanal, tert-butyl (2S)-2-formylpyrrolidine-1-carboxylate and 2-methyl-2-(morpholin-4-yl)propanal are commercially available. Ethoxy-2-methylpropanal was prepared from isobutyraldehyde as described in PCT Int. Appl., 2007142576. Compound 9 can also be condensed with a precursor group of R^{c}CHO and then converted to a compound of Formula (I). For example, 9 can be condensed with tert-butyl (1-formylcyclopropyl)-carbamate (prepared by oxidation of tert-butyl (1-(hydroxymethyl)cyclopropyl)carbamate see Bioorg. Med. Chem. Lett., 2008, 18 (6), 2188, with Dess Martin periodinane) or tert-butyl 2-methyl-1-oxopropan-2-ylcarbamate following by removal of the amino protecting group to give a compound of Formula (I) where R^{c} is 1-aminocycloprop-1-yl or 2-aminopropan-2-yl.

The condensation reaction can be also be carried out by adding the desired aldehyde with a base such as pyrrolidine or piperidine with or without chlorotrimethylsilane in dichloromethane or other suitable solvent (e.g. dioxane and ethanol). Alternatively, compounds of Formula (I) where X is —CO— can be prepared by reacting compound 8 with an acid of formula 10 where R^{c} is as defined in the Summary under amide coupling conditions.

Compounds of Formula (I) where Y is —SO₂— can be prepared by reacting 8 with a sulfonyl chloride of formula 11, followed by condensation of resulting compound 12 with an aldehyde of formula R^{c}HO as described above.

Compounds of Formula (I) where X is -alkyleneNR^{a}—, -cycloalkyleneNR^{a}—, -phenyleneNR^{a}— and -alkylene-O— and Y is CO or SO₂ can be prepared by substituting

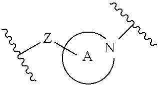

with amines of formula NH₂—X—NR^{a}PG¹ where PG¹ is a suitable amino protecting group, R^{a} is as defined in the Summary, and X is alkylene, cycloalkylene, or phenylene respectively, e.g., tert-butyl N-(2-aminoethyl)carbamate, tert-butyl N-[(2S)-1-hydroxypropan-2-yl]carbamate or NH₂—X—OPG² where PG² is a suitable hydroxy protecting group and X is alkylene, followed by steps described above.

### Utility

The compounds of Formula (I) and/or a pharmaceutically acceptable salt thereof are tyrosine kinase inhibitors, in particular BTK and hence are useful in the treatment of autoimmune disease, e.g., inflammatory bowel disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, Sjogren's syndrome including Sjogren's dry eye, non-Sjogren's dry eye, multiple sclerosis, Guillain-Barre syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, or vulvodynia.

The compounds of Formula (I) and/or a pharmaceutically acceptable salt thereof are also useful in the treatment of a heteroimmune condition or disease. In one embodiment of this aspect, the patient in need or recognized is suffering from a heteroimmune condition or disease, e.g., graft versus host disease, transplantation, transfusion, anaphylaxis, allergy, type I hypersensitivity, allergic conjunctivitis, allergic rhinitis, or atopic dermatitis.

In another embodiment of this aspect, the patient in need or recognized need is suffering from an inflammatory disease, e.g., asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In another embodiment of this aspect, the patient in need or recognized need is suffering from inflammatory skin disease which includes, by way of example, dermatitis, contact dermatitis, eczema, urticaria, rosacea, and scarring psoriatic lesions in the skin, joints, or other tissues or organs.

In yet another embodiment of this aspect, the patient in need or recognized need is suffering from a cancer. In one embodiment, the cancer is a B-cell proliferative disorder, e.g., diffuse large B cell lymphoma, follicular lymphoma, chronic lymphocytic lymphoma, chronic lymphocytic leukemia, B-ALL, B-cell prolymphocytic leukemia, SLL, multiple myeloma, lymphoplamascytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, the compound of Formula (I) and/or a pharmaceutically acceptable salt thereof is administered in combination with another an anti-cancer agent e.g., the anti-cancer agent is an inhibitor of mitogen-activated protein kinase signaling, e.g., . . . , gefinitinib or imatinib, ofatumumab, bendamustine, rituaximab, U0126, PD98059, PD184352, PD0325901, ARRY-142886, SB239063, SP600125, BAY 43-9006, wortmannin, Nexavar®, Tarceva®, Sutent®, Tykerb®, Sprycel®, Crizotinib, Xalkori®, or LY294002. When combination therapy is used, the agents can be administered simultaneously or sequentially. In yet another embodiment, the patient in need or recognized need is suffering from a thromboembolic disorder, e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In a fourth aspect, the disclosure is directed to use of compound of Formula (I) and/or a pharmaceutically acceptable salt thereof (and any embodiments thereof described herein) for use as a medicament. In one embodiment, the use of compound of Formula (I) and/or a pharmaceutically acceptable salt thereof is for treating inflammatory disease or proliferative diseases.

In a fifth aspect is the use of a compound of Formula (I) and/or a pharmaceutically acceptable salt thereof in the manufacture of a medicament for treating an inflammatory disease in a patient in which the activity of BTK or other tyrosine kinases contributes to the pathology and/or symptoms of the disease. In one embodiment of this aspect, the tyrosine kinase protein is BTK. In another embodiment of this aspect, the inflammatory disease is respiratory, cardiovascular, or proliferative diseases.

In any of the aforementioned aspects involving the treatment of proliferative disorders, including cancer, are further embodiments comprising administering the compound of Formula (I) and/or a pharmaceutically acceptable salt thereof in combination with at least one additional agent chosen from alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzamab, methotrexate, paclitaxel, Taxol™, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, dronabinol. When combination therapy is used, the agents can be administered simultaneously or sequentially.

### Testing

The kinase inhibitory activity of the compounds of the present disclosure can be tested by methods well known the art. The BTK inhibitory activity of the compounds and/or a pharmaceutically acceptable salt thereof of the present disclosure can be tested using the in vitro and in vivo assays described in Biological Examples 1-3 below. A determination of kinase inhibitory activity by any of those assays is considered to be kinase inhibitory activity within the scope of this disclosure even if any or all of the other assays do not result in a determination of kinase inhibitory activity.

Without being bound to any specific mechanistic theory, in those embodiments wherein the compound of the present disclosure is a reversible covalent inhibitor, it is believed that the cysteine sulfhydryl group and a carbon atom forming part of the carbon-carbon double bond in the group —X—Y—C(CN)═CHR^{c} (see Formula I) of the compound of the present disclosure can form a reversible, i.e., labile, covalent bond, such as wherein Cys 481 in BTK attacks an electron deficient carbon atom of the carbon-carbon double bond in the group —X—Y—C(CN)═CHR^{c} in the compound of present disclosure to form a thiol adduct (e.g., Michael reaction with cysteine).

In some embodiments, the electron deficient carbon atom of the olefin is distal to the carbon attached to the cyano group and to the electron withdrawing —X—Y— or —Y— moiety (see Formula I,) in the compounds of the present disclosure. Therefore, the combination of the cyano and the “—X—Y—” or “Y” moieties and the olefinic moiety to which they are bonded in the compounds of the present disclosure can increase the reactivity of the olefin to form a thiol adduct with the active site cysteine residue in BTK.

The compounds of the present disclosure bind with BTK in two different manners. In addition to the labile covalent binding, discussed above, they also form non-covalent bindi (e.g., via van der Waals binding, hydrogen binding, hydrophobic binding, hydrophilic binding, and/or electrostatic charge binding) with BTK, the non-covalent binding being sufficient to at least partially inhibit the kinase activity of the BTK.

As disclosed herein, the labile covalent binding between the compound of the disclosure and BTK occurs between the olefin in the inhibitor and the cysteine 481 residue thiol side chain at or near the site where the compound has the aforementioned non-covalent binding with the BTK.

As is evident, the compounds of the present disclosure which are reversible covalent inhibitors have both a cysteine-mediated covalent binding and a non-covalent binding with the BTK. This is in contrast with non-covalent reversible inhibitors which inhibit the BTK only via non-covalent binding and lack the cysteine-mediated covalent binding.

The result of the binding of the compounds of the present disclosure with BTK in the two different manners is a reversible covalent inhibitor having a slow off-rate and a protracted duration of action, in some instances comparable to an irreversible covalent inhibitor without forming permanent irreversible protein adducts. The difference between irreversible and reversible covalent inhibitors, particularly the compounds disclosed herein, can be ascertained utilizing assays disclosed herein.

In general, the binding involved in an inhibitor that forms a reversible covalent bond with BTK, i.e., the compounds disclosed herein, is stable when the btk is in certain configurations and susceptible to being broken when the BTK is in different configurations (in both cases under physiologic conditions), whereas the interaction between an inhibitor that forms an irreversible covalent bond is stable under physiologic conditions even when the BTK is in different configurations.

A reversible covalent bond often imparts unique properties related to the residence time of the compound within the cysteine-containing binding site. In this context, residence time refers to the temporal duration of the compound-target complex under different conditions (see Copeland R A, Pompliano D L, Meek T D. Drug-target residence time and its implications for lead optimization. Nat. Rev. Drug Discov. 5 (9), 730-739 (2006). The presence of a reversible covalent bond in a reversible covalent inhibitor as disclosed herein can lead to an extended residence time when compared to a compound that does not form a covalent bond with BTK. In one embodiment disclosed herein the compounds of the present disclosure that are reversible covalent inhibitors have a residence time of at least about 1 h. Residence time may be measured using an occupancy assay in a biochemical or cellular environment (see Biological Example 6 below). Additionally, residence time may be measured using a functional assay following a defined wash-out period.

Compounds that form an irreversible covalent bond in an irreversible covalent inhibitor share these extended residence time properties but may nonetheless be differentiated from reversible covalent inhibitor using a reversibility assay. The ability of the compound of the disclosure to form reversible covalent bond with Cys481 of BTK (UniprotKB Sequence ID Q06187) and the olefinic bond in the compound of the disclosure, can be determined by the assays described in Biological Examples 4-7 below. A determination of the binding reversibility of the covalent bond between the cysteine residue and the olefinic bond of the compound of the disclosure by any of Biological Examples 4-7 below is considered to be binding reversibility within the scope of this disclosure even if one or both of the other methods does not result in a determination of binding reversibility.

### Administration and Pharmaceutical Composition

In general, the compounds of this disclosure will be administered in a therapeutically effective amount by any of the accepted modes of administration for agents that serve similar utilities. Therapeutically effective amounts of the compounds disclosed herein may range from about 0.01 to about 500 mg per kg patient body weight per day, which can be administered in single or multiple doses. A suitable dosage level may be about 0.01 to about 250 mg/kg per day, about 0.05 to about 100 mg/kg per day, or about 0.1 to about 50 mg/kg per day. Within this range, the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount administered of the compound and/or a pharmaceutically acceptable salt thereof of this disclosure, i.e., the active ingredient, will depend upon numerous factors such as the severity of the disease to be treated, the age and relative health of the patient, the potency of the compound and/or pharmaceutically acceptable salt thereof being utilized, the route and form of administration, and other factors.

In general, compounds and/or pharmaceutically acceptable salts of this disclosure will be administered as pharmaceutical compositions by any one of the following routes: oral, systemic (e.g., transdermal, intranasal or by suppository), topically, or parenteral (e.g., intramuscular, intravenous or subcutaneous) administration. The preferred manner of administration is oral using a convenient daily dosage regimen, which can be adjusted according to the degree of affliction. Compositions can take the form of tablets, capsules, semisolids, powders, sustained release formulations, enteric coated or delayed release formulation, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, a compound and/or pharmaceutically acceptable salt disclosed herein in combination with at least one pharmaceutically acceptable excipient such as binders, surfactants, diluents, buffering agents, antiadherents, glidants, hydrophilic or hydrophobic polymers, retardants, stabilizing agents or stabilizers, disintegrants or superdisintegrants, antioxidants, antifoaming agents, fillers, flavors, colors, lubricants, sorbents, preservatives, plasticizers, and sweeteners. Acceptable excipients are non-toxic, aid administration, and do not adversely affect the therapeutic benefit of the compound disclosed herein. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols.

The compounds and/or pharmaceutically acceptable salt of the present disclosure can also be administered intranasally. Intranasal formulations are known in the art e.g., see U.S. Pat. Nos. 4,476,116, 5,116,817 and 6,391,452, each of which is incorporated herein by reference. The choice of excipients will depend upon the nature of the nasal dosage form e.g., solutions, suspensions, or powder. For administration by inhalation, the compounds and/or pharmaceutically acceptable salts of the present disclosure may be in the form of solutions, suspensions, and powders. These formulations are administered as an aerosol, a mist, or a powder and can be delivered from pressurized packs or a nebulizer with a suitable propellant such as dichlorodifluoromethane, trichlorofluoromethane, nitrogen, carbon dioxide, etc. In the case of a pressurized aerosol, the dosage unit may be determined by providing a valve to deliver a metered amount. Capsules and cartridges for use in an inhaler may be formulated containing a powder mix of the compound disclosed herein and a suitable powder base such as lactose or starch.

Topical formulation can be liquids, suspension, emulsions, and the like, and can be prepared by methods well known in the art. The formulation will contain, on a weight percent (wt %) basis, from about 0.01-99.99 wt % of a compound and/or pharmaceutically acceptable salt disclosed herein based on the total formulation, with the balance being one or more suitable pharmaceutical excipients and can be administered in single or multiple doses.

Suitable excipients include polymers, surfactants, buffering or pH adjusting agents, tonicity and osmotic adjusting agent(s), preservatives, and dispersing agents.

Other suitable pharmaceutical excipients and their formulations are described in Remington's Pharmaceutical Sciences, edited by E. W. Martin (Mack Publishing Company, 20^{th} ed., 2000).

The level of the compound in a formulation can vary within the full range employed by those skilled in the art. Typically, the formulation will contain, on a weight percent (wt %) basis, from about 0.01-99.99 wt % of a compound and/or pharmaceutically acceptable salt disclosed herein based on the total formulation, with the balance being one or more suitable pharmaceutical excipients.

The compounds and/or pharmaceutically acceptable salts of the present disclosure may be used in combination with one or more other drugs in the treatment of diseases or conditions for which compounds of the present disclosure or the other drugs may have utility, where the combination of the drugs together are safer or more effective than either drug alone. Such other drug(s) may be administered, by a route and in an amount commonly used therefore, contemporaneously or sequentially with a compound of the present disclosure. When a compound and/or pharmaceutically acceptable salt of the present disclosure is used contemporaneously with one or more other drugs, a pharmaceutical composition in unit dosage form containing such other drugs and the compound and/or pharmaceutically acceptable salt of the present disclosure is preferred. However, the combination therapy may also include therapies in which the compound and/or pharmaceutically acceptable salt of the present disclosure and one or more other drugs are administered on different overlapping schedules. It is also contemplated that when used in combination with one or more other active ingredients, the compounds and/or pharmaceutically acceptable salts of the present disclosure and the other active ingredients may be used in lower doses than when each is used singly.

Accordingly, the pharmaceutical compositions of the present disclosure also include those that contain one or more other active ingredients, in addition to a compound and/or pharmaceutically acceptable salt of the present disclosure.

The above combinations include combinations of a compound of the present disclosure not only with one other active compound, but also with two or more other active compounds. Likewise, compounds and/or pharmaceutically acceptable salts of the present disclosure may be used in combination with other drugs that are used in the prevention, treatment, control, amelioration, or reduction of risk of the diseases or conditions for which compounds of the present disclosure are useful. Such other drugs may be administered, by a route and in an amount commonly used therefore by those skilled in the art, contemporaneously or sequentially with a compound and/or pharmaceutically acceptable salt of the present disclosure. When a compound and/or pharmaceutically acceptable salt of the present disclosure is used contemporaneously with one or more other drugs, a pharmaceutical composition containing such other drugs in addition to the compound and/or pharmaceutically acceptable salt of the present disclosure is preferred. Accordingly, the pharmaceutical compositions of the present disclosure also include those that also contain one or more other active ingredients, in addition to a compound and/or pharmaceutically acceptable salt of the present disclosure. The weight ratio of the compound and/or pharmaceutically acceptable salt of the present disclosure to the second active ingredient may be varied and will depend upon the effective dose of each ingredient. Generally, an effective dose of each will be used.

Where the patient is suffering from or at risk of suffering from an autoimmune disease, an inflammatory disease, or an allergy disease, a compound and/or pharmaceutically acceptable salt of present disclosure can be used in with one or more of the following therapeutic agents in any combination: immunosuppressants (e.g., tacrolimus, -50-iethylstilb, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-.alpha. binding proteins (e.g., infliximab, etanercept, or adalimumab), abatacept, anakinra, interferon-.beta., interferon-.gamma., interleukin-2, allergy vaccines, antihistamines, antileukotrienes, beta-agonists, theophylline, and anticholinergics.

Where the patient is suffering from or at risk of suffering from a B-cell proliferative disorder (e.g., plasma cell myeloma), the patient can be treated with a compound and/or pharmaceutically acceptable salt disclosed herein in any combination with one or more other anti-cancer agents. In some embodiments, one or more of the anti-cancer agents are proapoptotic agents. Examples of anti-cancer agents include, but are not limited to, any of the following: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec™), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and docetaxol, such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents for use in combination with a compound disclosed herein include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

Other anti-cancer agents that can be employed in combination with a compound disclosed herein include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with a compound and/or pharmaceutically acceptable salt disclosed herein include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+−54-iethylstilbe cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; R.sub.11 retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with a compound disclosed herein include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products useful in combination with a compound or a pharmaceutically acceptable salt disclosed herein include but are not limited to vinca alkaloids (e.g., -55-iethylstil, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination a compound or a pharmaceutically acceptable salt disclosed herein include, but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxuridine, Cytarabine), purine analogs (e.g., mercaptopurine, thioguanine, pentostatin.

Examples of hormones and antagonists useful in combination a compound or a pharmaceutically acceptable salt disclosed herein include, but are not limited to, adrenocorticosteroids (e.g., prednisone), progestins (e.g., hydroxyprogesterone caproate, megestrol acetate, medroxyprogesterone acetate), estrogens (e.g., -56-iethylstilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which can be used in combination with an BTK inhibitor compound and/or a pharmaceutically acceptable salt of the disclosure include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B. Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Where the patient is suffering from or at risk of suffering from a thromboembolic disorder (e.g., stroke), the patient can be treated with a compound and/or pharmaceutically acceptable salt disclosed herein in any combination with one or more other anti-thromboembolic agents. Examples of anti-thromboembolic agents include, but are not limited any of the following: thrombolytic agents (e.g., alteplase anistreplase, streptokinase, urokinase, or tissue plasminogen activator), heparin, tinzaparin, warfarin, dabigatran (e.g., dabigatran etexilate), factor Xa inhibitors (e.g., fondaparinux, draparinux, rivaroxaban, DX-9065a, otamixaban, LY517717, or YM150), ticlopidine, clopidogrel, CS-747 (prasugrel, LY640315), ximelagatran, or BIBR 1048.

### EXAMPLES

The following preparations of compounds of Formula (I) and intermediates (References) are given to enable those skilled in the art to more clearly understand and to practice the present disclosure. They should not be considered as limiting the scope of the disclosure, but merely as being illustrative and representative thereof. The [symbol] line at the alkene carbon, in the compounds below denotes that the compounds are isolated as an undefined mixture of (E) and (Z) isomers.

### Synthetic Examples

### Reference 1

### Synthesis of (S)-6-amino-7-(4-phenoxyphenyl)-9-(pyrrolidin-2-ylmethyl)-7H-purin-8(9H)-one hydrochloride

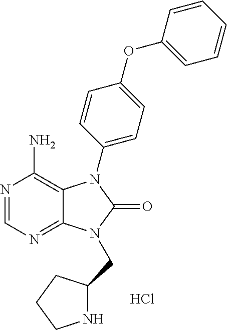 [SMILES: Nc1ncnc2c1n(-c1ccc(Oc3ccccc3)cc1)c(=O)n2C[C@@H]1CCCN1 ; Cl]

### Step 1

To a 500 ml three neck round bottomed flask, 4,6-dichloro-5-nitropyrimidine (25 g, 129 mmole) was dissolved in CH₂Cl₂ (300 ml) and cooled to 0° C. To this, dibenzylamine (49.6 ml, 257.7 mmole) was added dropwise by maintaining temperature at 0° C. and stirred for 1.5 h at same temperature. The reaction mixture was diluted with CH₂Cl₂ and washed with water. The combined organic layer was dried over sodium sulphate and concentrated to give the crude product which was purified by trituration with n-pentane to yield 38 g of N,N-dibenzyl-6-chloro-5-nitropyrimidin-4-amine (83.06% yield).

### Step 2

To a 250 ml three neck round bottomed flask under nitrogen atmosphere, N,N-dibenzyl-6-chloro-5-nitropyrimidin-4-amine (15 g, 42 mmole) was dissolved in 1,4-dioxane (150 ml) followed by addition of TEA (17.7 ml, 127 mmole) and stirred at rt for 10 minutes. (S)-tert-Butyl 2-(aminomethyl)pyrrolidine-1-carboxylate (12.7 g, 63.4 mmole) was added dropwise to the reaction mixture and stirred at room temperature for 3 h. The reaction mixture was concentrated, diluted with water and extracted with ethyl acetate. The combined organic layer was washed with brine. The organic layer was dried over sodium sulfate and concentrated to yield 20.9 g of (S)-tert-butyl 2-((6-(dibenzylamino)-5-nitropyrimidin-4-ylamino)methyl)pyrrolidine-1-carboxylate.

### Step 3

To a 500 ml three neck round bottomed flask, (S)-tert-butyl 2-((6-(dibenzylamino)-5-nitropyrimidin-4-yl amino)methyl)pyrrolidine-1-carboxylate (20.8 g, 40.1 mmole) was dissolved in ethyl acetate (200 ml) and a solution of ammonium chloride (10.72 g, 200.55 mmole) in water (150 ml). To this, zinc dust (13 g, 200.55 mmole) was added at 10° C. and stirred at room temperature for 2.5 h. The reaction mixture was filtered off and the aqueous layer was extracted with ethyl acetate. The combined organic layer was dried over sodium sulfate and concentrated. The crude mixture was purified using column purification to yield 14.3 g of (S)-tert-butyl 2-((5-amino-6-(dibenzylamino)-pyrimidin-4-ylamino)methyl)pyrrolidine-1-carboxylate.

### Step 4

To a 250 ml sealed tube under nitrogen atmosphere, (S)-tert-butyl 2-((5-amino-6-(dibenzylamino)pyrimidin-4-ylamino)methyl)pyrrolidine-1-carboxylate (13.5 g, 27.6 mmole), 1,1′-carbonyldiimidazole (17.9 g, 110 mmole) and DIPEA (19.3 ml, 110 mmole) were dissolved in dry dichloroethane (140 ml) at room temperature and then stirred at 100° C. for 2 h. The reaction mixture was diluted with water and extracted with CH₂Cl₂. The combined organic layer was washed with brine, dried over sodium sulfate and concentrated to give crude product which was purified using column purification, with 50% ethyl acetate in hexanes to yield 10 g of (S)-tert-butyl 2-((6-(dibenzylamino)-8-oxo-7H-purin-9(8H)-yl)methyl)pyrrolidine-1-carboxylate.

### Step 5

To a 250 ml autoclave vessel under nitrogen atmosphere, (S)-tert-butyl 2-((6-(dibenzylamino)-8-oxo-7H-purin-9(8H)-yl)methyl)pyrrolidine-1-carboxylate (3.6 g, 7.0 mmole) was dissolved in glacial acetic acid (90 ml) and 10% dry palladium on carbon (1.8 g) was added at RT. The reaction mixture was stirred at 90° C. with 35 Kg of hydrogen pressure for 2.5 h. After completion of the reaction, reaction mixture was filtered through high flow and washed with methanol. The filtrate was concentrated under vacuum and basified with 5N sodium hydroxide solution (100 ml). The aqueous layer was extracted with ethyl acetate and the combined organic layer was dried over sodium sulfate and concentrated to yield 1.5 g of (S)-tert-butyl-2-((6-amino-8-oxo-7H-purin-9(8H)-yl)methyl)pyrrolidine-1-carboxylate.

### Step 6

To a 500 ml round bottomed flask, (S)-tert-butyl 2-((6-amino-8-oxo-7H-purin-9(8H)-yl)methyl)pyrrolidine-1-carboxylate (1.8 g, 5.38 mmole) and 4-phenoxyphenylboronic acid (3.45 g, 16.14 mmole) were dissolved in CH₂Cl₂ (100 ml) followed by dropwise addition of pyridine (1.29 ml, 16.14 mmole). To this, copper (II) acetate (0.98 g, 5.38 mmole) was added at room temperature. The reaction mixture was stirred at room temperature for 16 h under oxygen atmosphere. The reaction mixture was diluted with water and extracted with CH₂Cl₂. The organic layer was dried over sodium sulfate and concentrated to give crude product which was purified by column chromatography, eluting the compound with 2% methanol in CH₂Cl₂ to yield 0.65 g of (S)-tert-butyl 2-((6-amino-8-oxo-7-(4-phenoxyphenyl)-7H-purin-9(8H)-yl)methyl)pyrrolidine-1-carboxylate.

### Step 7

To a 250 ml round bottomed flask under nitrogen atmosphere, (S)-tert-butyl 2-((6-amino-8-oxo-7-(4-phenoxyphenyl)-7H-purin-9(8H)-yl)methyl)pyrrolidine-1-carboxylate (1.35 g, 2.68 mmole) was dissolved in 1,4-dioxane (15 ml) followed by dropwise addition of 5N HCl in dioxane (55 ml) at 15° C. Reaction mixture was stirred at room temperature for 4 h. The reaction mixture was concentrated under vacuum and the solid thus obtained was triturated with acetone (25 ml) to yield 1.05 g of (S)-6-amino-7-(4-phenoxyphenyl)-9-(pyrrolidin-2-ylmethyl)-7H-purin-8(9H)-one hydrochloride.

### Reference 2

### Synthesis of 2-cyano-4,4-dimethylpent-2-enoic acid

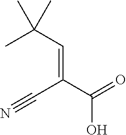

To a 25 ml sealed tube were dissolved 2-cyanoacetic acid (5 g, 58.8 mmole), pivaldehyde (10.1 g, 118 mmole) and piperidine (6.38 ml, 64.7 mmole) in methanol (100 ml). The reaction mixture was heated at 100° C. for 3 h. After 3 h, the reaction mixture was concentrated under vacuum, diluted with water, washed with CH₂Cl₂ and organic layer discarded. The aqueous layer was acidified with diluted HCl and extracted with CH₂Cl₂. The combined organic layer was dried over sodium sulfate and concentrated under vacuum to yield 1.8 g of 2-cyano-4,4-dimethylpent-2-enoic acid.

### Reference 3

### Synthesis of 6-amino-7-(4-phenoxyphenyl)-9-(pyrrolidin-3-yl)-7H-purin-8(9H)-one hydrochloride

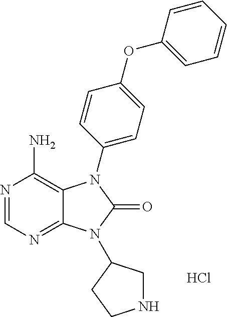 [SMILES: Nc1ncnc2c1n(-c1ccc(Oc3ccccc3)cc1)c(=O)n2C1CCNC1 ; Cl]

### Step 1

To a 250 ml three neck round bottomed flask under nitrogen atmosphere, N,N-dibenzyl-6-chloro-5-nitro pyrimidin-4-amine (5.6 g, 15.8 mmole) prepared as in Step 1, Reference 1 was dissolved in 1,4-dioxane (100 ml) followed by addition of TEA (4.8 g, 47 mmole) and stirred at rt for 10 minutes. tert-Butyl 3-aminopyrrolidine-1-carboxylate (4.41 g, 23.7 mmole) was added dropwise to the reaction mixture and stirred at 50° C. for 2 h. The reaction mixture was concentrated and diluted with water and extracted with ethyl acetate. The combined organic layer was washed with brine. Organic layer was dried over sodium sulfate and concentrated to yield 8 g (crude) of tert-butyl 3-(6-(dibenzylamino)-5-nitropyrimidin-4-ylamino)pyrrolidine-1-carboxylate.

### Step 2

To a 1 liter three neck round bottomed flask, tert-butyl 3-(6-(dibenzylamino)-5-nitropyrimidin-4-ylamino)pyrrolidine-1-carboxylate (8 g, 15.8 mmole) was dissolved in ethyl acetate (125 ml) and saturated ammonium chloride (250 ml). To this, zinc dust (5.15 g, 79.23 mmole) was added at 10° C. and stirred at room temperature for 2 h. The reaction mixture was filtered and the aqueous layer was extracted with ethyl acetate. The combined organic layer was dried over sodium sulfate and concentrated to yield 7 g of tert-butyl3-(5-amino-6-(dibenzylamino)pyrimidin-4-ylamino)pyrrolidine-1-carboxylate which was used without further purification.

### Step 3

To a 125 ml sealed tube under nitrogen atmosphere, tert-butyl 3-(5-amino-6-(dibenzylamino)pyrimidin-4-ylamino)pyrrolidine-1-carboxylate (3 g, 6.3 mmole), 1,1′-carbonyldiimidazole (4.1 g, 25.2 mmole) and DIPEA (3.23 g, 24.9 mmole) were dissolved in dry THF (50 ml) at room temperature and stirred at 100° C. for 1 h. The reaction mixture was concentrated and diluted with water and extracted with ethyl acetate. The combined organic layer was washed with brine. The organic layer was dried over sodium sulfate and concentrated to give crude product which was purified using column purification by eluting the compound with 25% ethyl acetate in hexanes to yield 2 g of tert-butyl 3-(6-(dibenzylamino)-8-oxo-7H-purin-9(8H)-yl)pyrrolidine-1-carboxylate.

### Step 4

To a 250 ml auto clave reactor under nitrogen atmosphere, tert-butyl 3-(6-(dibenzylamino)-8-oxo-7H-purin-9(8H)-yl)pyrrolidine-1-carboxylate (2 g, 3.99 mmole) was dissolved in glacial acetic acid (80 ml) and 10% dry palladium on carbon was added at rt. The reaction mixture was stirred at 80° C. with 35 Kg of hydrogen pressure for 2 h. The reaction mixture was filtered and the filtrate was concentrated under vacuum and basified with saturated sodium bicarbonate solution. The aqueous layer was extracted with CH₂Cl₂. The combined organic layer was dried over sodium sulfate and concentrated to yield 1.1 g of tert-butyl 3-(6-amino-8-oxo-7H-purin-9(8H)-yl)pyrrolidine-1-carboxylate which was used without further purification.

### Step 5

To a 250 ml three neck round bottomed flask, tert-butyl 3-(6-amino-8-oxo-7H-purin-9(8H)-yl)pyrrolidine-1-carboxylate (1.1 g, 3.4 mmole) and 4-phenoxyphenylboronic acid (2.2 g, 10.3 mmole) were dissolved in CH₂Cl₂ (100 ml) followed by dropwise addition of pyridine (0.8 g, 10.1 mmole). To this, copper (II) acetate (0.62 g, 3.4 mmole) was added at rt under argon atmosphere. Reaction mixture was stirred at rt for 16 h under oxygen atmosphere. The reaction mixture was diluted with water, extracted with CH₂C2. The organic layer was dried over sodium sulfate and concentrated to give crude product which was purified using column purification by eluting the compound with 2% methanol in CH₂Cl₂ to yield 0.75 g of tert-butyl 3-(6-amino-8-oxo-7-(4-phenoxy phenyl)-7H-purin-9(8H)-yl)pyrrolidine-1-carboxylate.

### Step 6

To a 50 ml one neck round bottomed flask under nitrogen atmosphere, tert-butyl 3-(6-amino-8-oxo-7-(4-phenoxyphenyl)-7H-purin-9(8H)-yl)Pyrrolidine-1-carboxylate (0.75 g, 1.53 mmole) was dissolved in 1,4 dioxane (5 ml) followed by dropwise addition of 5N HCl in dioxane (15 ml) at 15° C. Reaction mixture was stirred at rt for 3 h. After completion of the reaction, reaction mixture was concentrated under vacuum and the solid thus obtained was triturated with acetone (25 ml) to yield 0.65 g of 6-amino-7-(4-phenoxyphenyl)-9-(pyrrolidin-3-yl)-7H-purin-8(9H)-one hydrochloride which was used without further purification.

Proceeding as described above but substituting tert-butyl 3-aminopyrrolidine-1-carboxylate with tert-butyl (R)-3-aminopyrrolidine-1-carboxylate, (R)-6-amino-7-(4-phenoxyphenyl)-9-(pyrrolidin-3-yl)-7H-purin-8(9H)-one was prepared.

### Reference 4

### Synthesis of 2-[4-(3,4-dichlorophenoxy)-3-methoxyphenyl]-4,4,5,5-tetramethyl-1,3,2-dioxaborolane

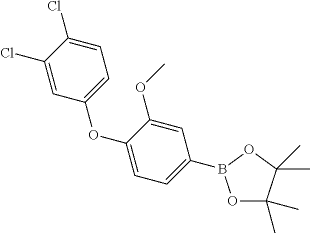 [SMILES: COc1cc(B2OC(C)(C)C(C)(C)O2)ccc1Oc1ccc(Cl)c(Cl)c1]

### Step 1

A mixture of 3,4-dichlorophenol (38 g, 233.13 mmol, 1.00 equiv), 1-fluoro-2-methoxy-4-nitrobenzene (40 g, 233.75 mmol, 1.00 equiv) and potassium carbonate (64 g, 463.77 mmol, 1.99 equiv) in N,N-dimethylformamide (250 mL) was stirred overnight at 60° C. The resulting solution was diluted with 1000 mL of water, extracted with 3×200 mL of ethyl acetate and the organic layers combined. The resulting mixture was washed with 3×500 mL of brine. The mixture was dried over anhydrous sodium sulfate and concentrated under vacuum to yield 60 g (82%) of 1,2-dichloro-4-(2-methoxy-4-nitrophenoxy)benzene as a brown solid.

### Step 2

A mixture of 1,2-dichloro-4-(2-methoxy-4-nitrophenoxy)benzene (60 g, 190.40 mmol, 1.00 equiv), Fe (53 g, 946.43 mmol, 4.97 equiv) and ammonium chloride (10 g, 188.68 mmol, 0.99 equiv) in tetrahydrofuran/water (1/2) (600 mL) was stirred overnight at 60° C. under an inert atmosphere of nitrogen. The mixture was filtered through Celite and the filtrate was concentrated under vacuum. The resulting solution was extracted with 3×500 mL of dichloromethane and the organic layers combined. The resulting mixture was washed with 3×500 mL of brine. The mixture was dried over anhydrous magnesium sulfate and concentrated under vacuum to give 40 g (74%) of 4-(3,4-dichlorophenoxy)-3-methoxyaniline as a light gray solid.

### Step 3

A solution of sodium nitrite (14.4 g, 208.70 mmol, 1.98 equiv) in water (500 mL) was added dropwise into a solution of 4-(3,4-dichlorophenoxy)-3-methoxyaniline (30 g, 105.58 mmol, 1.00 equiv) in sulfuric acid (1000 mL) with stirring at 0° C. and the mixture was stirred for 30 min at 0° C. The above mixture was added dropwise to a solution of potassium iodide (1000 mL, 5%) in water with stirring at 50° C. The reaction was completed immediately. The reaction mixture was cooled to room temperature, extracted with 3×500 mL of ethyl acetate and the organic layers combined. The resulting mixture was washed with 3×500 mL of saturated aqueous sodium bicarbonate and 3×500 mL of brine. The mixture was dried over anhydrous sodium sulfate and concentrated under vacuum to give 24 g (crude) of 1,2-dichloro-4-(4-iodo-2-methoxyphenoxy)benzene as red oil.

### Step 4

A mixture of 1,2-dichloro-4-(4-iodo-2-methoxyphenoxy)benzene (93 g, 235.43 mmol, 1.00 equiv) in 1,4-dioxane (500 mL), potassium acetate (46 g, 469.39 mmol, 1.99 equiv), 4,4,5,5-tetramethyl-2-(4,4,5,5-tetramethyl-1,3,2-dioxaborolan-2-yl)-1,3,2-dioxaborolane (89 g, 350.39 mmol, 1.49 equiv) and Pd(dppf)Cl₂ (4.65 g) was stirred overnight at 90° C. under an inert atmosphere of nitrogen. The reaction mixture was cooled to room temperature and concentrated under vacuum. The residue was dissolved in 500 mL of ethyl acetate and washed with mL of water and brine. The mixture was dried over anhydrous sodium sulfate and concentrated under vacuum. The residue was applied onto a silica gel column with ethyl acetate/petroleum ether (1/100) to yield 10 g (11%) of 2-[4-(3,4-dichlorophenoxy)-3-methoxyphenyl]-4,4,5,5-tetramethyl-1,3,2-dioxaborolane as light yellow oil.

### Reference 5

### Synthesis of 2-[4-(2,6-difluorophenoxy)phenyl]-4,4,5,5-tetramethyl-1,3,2-dioxaborolane

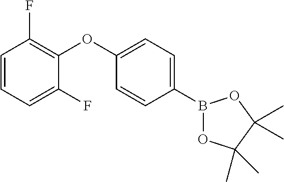 [SMILES: CC1(C)OB(c2ccc(Oc3c(F)cccc3F)cc2)OC1(C)C]

### Step 1

Into a 500-mL 4-necked round-bottom flask, was placed a solution of sodium hydride (4.05 g, 168.75 mmol, 1.70 equiv) in N,N-dimethylformamide (200 mL). A solution of 1-fluoro-4-nitrobenzene (14 g, 99.22 mmol, 1.00 equiv) in N,N-dimethylformamide (50 mL) was added dropwise with stirring at 0° C. over 20 min. The resulting solution was stirred for 2 hr at room temperature. Cu₂Cl₂ (9.83 g, 100.31 mmol, 1.01 equiv) was added and a solution of 2,6-difluorophenol (15.5 g, 119.15 mmol, 1.20 equiv) in N,N-dimethylformamide (50 mL) was added dropwise with stirring at 25° C. over 10 min. The resulting solution was stirred for 12 h at 100° C. in an oil bath, diluted with 500 mL of water and extracted with ethyl acetate. The combined organic layers were washed with water and brine, dried over anhydrous sodium sulfate and concentrated under vacuum. The residue was placed on a silica gel column and eluted with ethyl acetate/petroleum ether (1/8) to give 20 g (80%) of 1,3-difluoro-2-(4-nitrophenoxy)benzene as brown oil.

### Step 2

Into a 500 mL, 3-necked round-bottom flask purged and maintained under an inert atmosphere of nitrogen, was placed a solution of 1,3-difluoro-2-(4-nitrophenoxy)benzene (20 g, 79.62 mmol, 1.00 equiv) in methanol (200 mL), Raney Nickel (2 g). A solution of hydrazine hydrate (12.67 g) in methanol (50 mL) was added dropwise with stirring in 15 min. The resulting solution was stirred for 12 h at 25° C., then filtrated and the filtrate was concentrated under vacuum. The residue was diluted with f ethyl acetate, washed with water and brine, and dried over anhydrous sodium sulfate and concentrated under vacuum to give 16 g (91%) of 4-(2,6-difluorophenoxy)aniline as black oil.

### Step 3

Into a 250-mL 4-necked round-bottom flask, was placed 4-(2,6-difluorophenoxy)-aniline (8.84 g, 39.96 mmol, 1.00 equiv), hydrogen chloride (37%) (10.14 g, 277.81 mmol, 6.95 equiv) and water (20 mL). NaNO₂ (3.04 g, 44.06 mmol, 1.10 equiv) in water (10 mL) was added dropwise with stirring at 0° C. over 5 min., and the reaction mixture was stirred for 30 mins at 0° C. The reaction mixture was added into a solution of NaI (18 g, 120.00 mmol, 3.00 equiv) in water (20 mL) at 25° C. in batches over 5 min. The resulting solution was stirred for 2 h at 25° C. and then extracted with of ethyl acetate and the organic layers were combined. The combined organic layers were washed with water and brine, dried over anhydrous sodium sulfate and concentrated under vacuum to give 10.2 g (77%) of 1,3-difluoro-2-(4-iodophenoxy)benzene as brown oil.

### Step 4

Into a 100 mL 3-necked round-bottom flask purged and maintained in an inert atmosphere of nitrogen, was placed a solution of 1,3-difluoro-2-(4-iodophenoxy)benzene (2 g, 6.02 mmol, 1.00 equiv) in N,N-dimethylformamide (50 mL), 4,4,5,5-tetramethyl-2-(tetramethyl-1,3,2-dioxaborolan-2-yl)-1,3,2-dioxaborolane (1.68 g, 6.62 mmol, 1.10 equiv), potassium acetate (1.76 g, 17.93 mmol, 3.0 equiv), and Pd(OAc)₂ (68 mg, 0.30 mmol, 0.05 equiv). The resulting solution was stirred for 12 h at 85° C. in an oil bath. The reaction mixture was then quenched with water. The resulting solution was extracted with ethyl acetate and the organic layers combined and washed with water and brine. The organics were dried over anhydrous sodium sulfate and concentrated under vacuum. The residue was loaded onto a silica gel column and eluted with ethyl acetate/petroleum ether (1/8) to give 1.5 g (75%) of 2-[4-(2,6-difluorophenoxy)phenyl]-4,4,5,5-tetramethyl-1,3,2-dioxaborolane as a light yellow solid.

### Reference 6

### Synthesis of (2-fluoro-4-phenoxyphenyl)-boronic acid

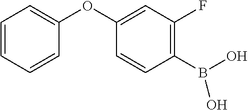 [SMILES: OB(O)c1ccc(Oc2ccccc2)cc1F]

### Step 1

Into a 250 mL round-bottom flask, was placed a solution of 4-bromo-3-fluorophenol (5 g, 26.18 mmol, 1.00 equiv) in dichloromethane (100 mL), phenylboronic acid (3.5 g, 28.70 mmol, 1.10 equiv), Cu(AcO)₂ (5.7 g), triethylamine (5.3 g), and 4 A molecular sieves (15 g). The resulting solution was stirred overnight at room temperature. The solids were filtered out. The filtrate was dried over anhydrous sodium sulfate and concentrated under vacuum. The residue was loaded onto a silica gel column and eluted with ethyl acetate/petroleum ether (1:100-1:50). This resulted in 2 g (29%) of 1-bromo-2-fluoro-4-phenoxybenzene as colorless oil.

### Step 2

Into a 100 mL 3-necked round-bottom flask purged and maintained under an inert atmosphere of nitrogen, was placed a solution of 1-bromo-2-fluoro-4-phenoxybenzene (2 g, 7.49 mmol, 1.00 equiv) in tetrahydrofuran (20 mL). BuLi (IM) (8 mL) was added dropwise with stirring at −70 to −80° C. The resulting solution was stirred for 30 min at −70-80° C. in a liquid nitrogen bath. Tris(propan-2-yl)borate (1.7 g, 9.04 mmol, 1.21 equiv) was added dropwise with stirring at −70 to −80° C. The resulting solution was allowed to react, with stirring, for an additional 2 h while the temperature was maintained at −70 to −80° C. The reaction was then quenched by the addition of 100 mL of water, extracted with ethyl acetate and the organic layers were combined and dried over anhydrous sodium sulfate and concentrated under vacuum. The residue was loaded onto a silica gel column and eluted with ethyl acetate/petroleum ether (1:20) to give 1.6 g (92%) of (2-fluoro-4-phenoxyphenyl)-boronic acid as a white solid.

### Reference 7

### Synthesis of 2-[4-(2,3-difluorophenoxy)phenyl]-4,4,5,5-tetramethyl-1,3,2-dioxaborolane

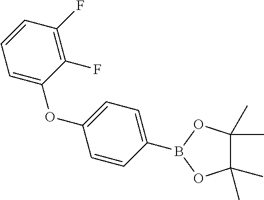 [SMILES: CC1(C)OB(c2ccc(Oc3cccc(F)c3F)cc2)OC1(C)C]

### Step 1

Into a 500 mL round-bottom flask, was placed a solution of (2,3-difluorophenyl)-boronic acid (30 g, 189.98 mmol, 1.00 equiv) in dichloromethane (250 mL). H₂O₂ (30 mL) was added dropwise with stirring. The resulting solution was stirred for 2 h at 25° C. The resulting mixture was washed with water and brine, dried over anhydrous magnesium sulfate and concentrated under vacuum to give 23 g (93%) of 2,3-difluorophenol as brown oil.

### Step 2

Into a 500 mL, 4-necked round-bottom flask purged and maintained under an inert atmosphere of nitrogen, was placed a solution of sodium hydride (6.8 g, 170.00 mmol, 1.70 equiv, 60%) in N,N-dimethylformamide (200 mL). A solution of 1-fluoro-4-nitrobenzene (14.1 g, 99.93 mmol, 1.00 equiv) in N,N-dimethylformamide (50 mL) was added dropwise with stirring at 0° C. in 15 min. The resulting solution was stirred for 2 h at room temperature. CuCl (10 g, 101.01 mmol, 1.00 equiv) was added and a solution of 2,3-difluorophenol (15.6 g, 119.91 mmol, 1.20 equiv) in N,N-dimethylformamide (50 mL) was added dropwise with stirring. The resulting solution was allowed to react, with stirring, for an additional 12 h while the temperature was maintained at 100° C. in an oil bath. The resulting solution was extracted with ether and the organic layers combined. The organic layers was washed with water and brine, dried over anhydrous sodium sulfate and concentrated under vacuum. The residue was loaded onto a silica gel column and eluted with ethyl acetate/petroleum ether (1:8) to give 21.2 g (84%) of 1,2-difluoro-3-(4-nitrophenoxy)benzene as a brown solid.

### Step 3

Into a 500 mL, 3-necked round-bottom flask purged and maintained under an inert atmosphere of nitrogen, was placed a solution of 1,2-difluoro-3-(4-nitrophenoxy)benzene (21.2 g, 84.40 mmol, 1.00 equiv) in methanol (200 mL), and Raney Nickel (2 g). A solution of hydrazine hydrate (12.67 g, 3.00 equiv) in methanol (50 mL) was added dropwise with stirring in 15 min. The resulting solution was stirred for 12 h at 25° C. The solids were filtered out and the filtrate was concentrated under vacuum. The residue was diluted with 200 mL of ethyl acetate and washed with water and brine, dried over anhydrous sodium sulfate and concentrated under vacuum to give 16.3 g (87%) of 4-(2,3-difluorophenoxy)aniline as black oil.

### Step 4

Into a 250-mL 4-necked round-bottom flask, was placed 4-(2,3-difluorophenoxy)-aniline (8.84 g, 39.96 mmol, 1.00 equiv), hydrogen chloride (10.14 g, 100.01 mmol, 2.50 equiv), and water (20 mL). A solution of NaNO₂ (3.04 g, 44.06 mmol, 1.10 equiv) in water (10 mL) was added dropwise with stirring in portions at 0° C. The mixture was stirred at 0° C. for half an hour. To this was added urea (1 g, 16.65 mmol). The mixture was stirred at 0° C. for 20 min and poured into the solution of NaI (18 g, 120.00 mmol, 3.00 equiv) in water (20 mL) at room temperature. The resulting solution was stirred at room temperature for 1 h and then extracted with ethyl acetate. The organic layers combined and dried over anhydrous sodium sulfate and concentrated under vacuum to give 10.5 g (79%) of 1,2-difluoro-3-(4-iodophenoxy)benzene as brown oil.

### Step 5

Into a 100 mL, 3-necked round-bottom flask purged and maintained under an inert atmosphere of nitrogen, was placed a solution of 1,2-difluoro-3-(4-iodophenoxy)benzene (2 g, 6.02 mmol, 1.00 equiv) in N,N-dimethylformamide (50 mL), 4,4,5,5-tetramethyl-2-(tetramethyl-1,3,2-dioxaborolan-2-yl)-1,3,2-dioxaborolane (1.68 g, 6.62 mmol, 1.10 equiv), potassium acetate (68 mg, 0.69 mmol, 0.05 equiv), and Pd(OAc)₂(1.76 g, 7.84 mmol, 3.00 equiv). The resulting solution was stirred for 12 h at 85° C. in an oil bath. The reaction was then diluted with water, extracted with ethyl acetate and the organic layers were combined. The organics were washed with water and brine, dried over anhydrous sodium sulfate and concentrated under vacuum. The residue was loaded onto a silica gel column and eluted with ethyl acetate/petroleum ether (1/8) to give 1.5 g (75%) of 2-[4-(2,3-difluorophenoxy)phenyl]-4,4,5,5-tetramethyl-1,3,2-dioxaborolane as a light yellow solid.

### Reference 8

### Synthesis of 2-[4-(3-fluorophenoxy)phenyl]-4,4,5,5-tetramethyl-1,3,2-dioxaborolane

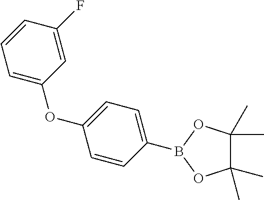 [SMILES: CC1(C)OB(c2ccc(Oc3cccc(F)c3)cc2)OC1(C)C]

Into a 250 mL round-bottom flask, was placed a solution of 4-(tetramethyl-1,3,2-dioxaborolan-2-yl)phenol (5 g, 22.72 mmol, 1.00 equiv) in dichloromethane (100 mL), (3-fluorophenyl)boronic acid (3.5 g, 25.01 mmol, 1.10 equiv), Cu(AcO)₂ (5 g), 4 A molecular sieves (15 g), and triethylamine (4.6 g). The resulting solution was stirred overnight at room temperature. The solids were filtered out and the filtrate was dried over anhydrous sodium sulfate and concentrated under vacuum. The residue was loaded onto a silica gel column and eluted with ethyl acetate/petroleum ether (1:100-1:50) to give 1.8 g (25%) of 2-[4-(3-fluorophenoxy)phenyl]-4,4,5,5-tetramethyl-1,3,2-dioxaborolane as a colorless oil.

### Example 1

### Synthesis of (S)-2-(2-((6-amino-8-oxo-7-(4-phenoxyphenyl)-7H-purin-9(8H)-yl)methyl)pyrrolidine-1-carbonyl)-4,4-dimethylpent-2-enenitrile

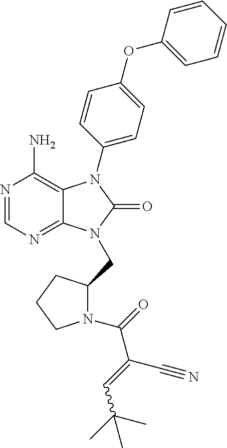 [SMILES: CC(C)(C)C=C(C#N)C(=O)N1CCC[C@H]1Cn1c(=O)n(-c2ccc(Oc3ccccc3)cc2)c2c(N)ncnc21]

To a 25 ml round bottomed flask under nitrogen atmosphere, 2-cyano-4,4-dimethylpent-2-enoic acid (56 mg, 0.37 mmole) was dissolved in DMF (3 ml) at rt. To this, HATU (140 mg, 0.37 mmole) was added at 0° C. and stirred at 0° C. for 30 min. A solution of (S)-6-amino-7-(4-phenoxyphenyl)-9-(pyrrolidin-2-ylmethyl)-7H-purin-8(9H)-one hydrochloride (150 mg, 0.34 mmole) in DMF (2 ml) was added dropwise at 0° C. followed by addition of DIPEA (0.35 ml, 2.04 mmole) at same temperature and the reaction mixture was stirred for 30 min at rt. After completion of the reaction, the reaction mixture was diluted with ethyl acetate and washed with brine solution followed by saturated sodium bicarbonate solution. The organic layer was dried over sodium sulfate and concentrated to give crude product which was purified by column chromatography, eluting with 2% methanol in CH₂Cl₂ to yield 52 mg of (S)-2-(2-((6-amino-8-oxo-7-(4-phenoxyphenyl)-7H-purin-9(8H)-yl)methyl)pyrrolidine-1-carbonyl)-4,4-dimethylpent-2-enenitrile. LC-MS (ES, m/z): 538.4 [M+H].

### Example 2

### Synthesis of (S)-2-(2-((6-amino-8-oxo-7-(4-phenoxyphenyl)-7H-purin-9(8H)-yl)methyl)-pyrrolidine-1-carbonyl)-4-methyl-4-morpholinopent-2-enenitrile

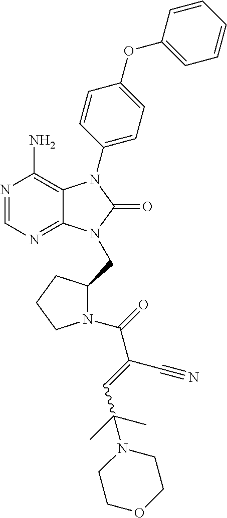 [SMILES: CC(C)(C=C(C#N)C(=O)N1CCC[C@H]1Cn1c(=O)n(-c2ccc(Oc3ccccc3)cc2)c2c(N)ncnc21)N1CCOCC1]

### Step 1

To a 30 ml vial under nitrogen atmosphere, 2-cyanoacetic acid (85 mg, 1 mmole) was dissolved in DMF (5 ml) at room temperature. To this, HATU (380 mg, 1 mmole) was added at 0° C. and stirred at 0° C. for 30 minutes. A solution of S)-6-amino-7-(4-phenoxyphenyl)-9-(pyrrolidin-2-ylmethyl)-7H-purin-8(9H)-one hydrochloride (400 mg, 0.91 mmole) in DMF (3 ml) was added dropwise at 0° C. followed by addition of DIPEA (0.95 ml, 5.46 mmole) at same temperature and the reaction mixture was stirred for 30 minutes at rt. The reaction mixture was diluted with ethyl acetate (25 ml) and washed with water (4×50 ml) followed by saturated sodium bicarbonate solution (50 ml). The organic layer was dried over sodium sulfate, concentrated to give crude product which was purified using trituration with n-pentane to yield 300 mg of (S)-3-(2-((6-amino-8-oxo-7-(4-phenoxyphenyl)-7H-purin-9(8H)-yl)methyl)pyrrolidin-1-yl)-3-oxopropanenitrile.

### Step 2

To a 50 ml single neck round bottomed flask under nitrogen atmosphere, (S)-3-(2-((6-amino-8-oxo-7-(4-phenoxyphenyl)-7H-purin-9(8H)-yl)methyl)pyrrolidin-1-yl)-3-oxopropanenitrile (0.22 g, 0.46 mmole) was dissolved in mixture of ACN (16 ml) and DMF (4 ml) at rt. To this, TMS-Cl (2.06 ml, 17.0 mmole) was added and stirred at same temperature for 5 min. Pyrrolidine (2.19 ml, 26.7 mmole) was added dropwise by maintaining temperature at 20-25° C. followed by addition of 2-methyl-2-morpholinopropanal (1.16 g, 7.36 mmole) at same temperature, the reaction mixture was stirred for 2 h rt. The reaction mixture was diluted by ethyl acetate (50 ml) and washed with water (4×50 ml) followed by brine solution (50 ml). The organic layer was dried over sodium sulfate and concentrated to give crude product which was purified by using column purification followed by prep HPLC to yield 74 mg of (S)-2-(2-((6-amino-8-oxo-7-(4-phenoxyphenyl)-7H-purin-9(8H)-yl)methyl)pyrrolidine-1-carbonyl)-4-methyl-4-morpholinopent-2-enenitrile. LC-MS (ES, m/z): 609.2 [M+H].

### Example 3

### Synthesis of 2-(3-(6-amino-8-oxo-7-(4-phenoxyphenyl)-7H-purin-9(8H)-yl)pyrrolidine-1-carbonyl)-4-methyl-4-morpholinopent-2-enenitrile

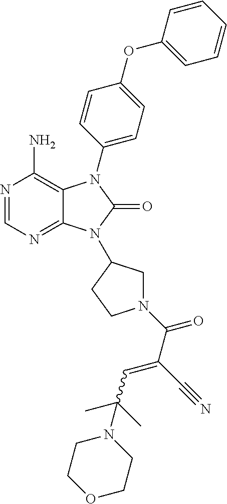 [SMILES: CC(C)(C=C(C#N)C(=O)N1CCC(n2c(=O)n(-c3ccc(Oc4ccccc4)cc3)c3c(N)ncnc32)C1)N1CCOCC1]

### Step 1

In a 30 ml vial under nitrogen atmosphere, 2-cyanoacetic acid (55 mg, 0.65 mmole) was dissolved in DMF (0.5 ml) at rt. To this, HATU (247 mg, 0.65 mmole) was added at 0° C. and stirred at 0° C. for 30 minutes. 6-Amino-7-(4-phenoxyphenyl)-9-(pyrrolidin-3-yl)-7H-purin-8(9H)-one hydrochloride (250 mg, 0.59 mmole) in DMF (0.5 ml) was added dropwise at 0° C. followed by addition of DIPEA (0.4 ml, 1.77 mmole) at same temperature and the reaction mixture was stirred for 30 minutes at rt. The reaction mixture was diluted by ethyl acetate and washed with water followed by saturated sodium bicarbonate solution. The organic layer was dried over sodium sulfate and concentrated to give crude product which was purified using column purification by eluting the crude with 1.4% methanol in CH₂Cl₂ to yield 0.21 g of 3-(3-(6-amino-8-oxo-7-(4-phenoxyphenyl)-7H-purin-9(8H)-yl)pyrrolidin-1-yl)-3-oxopropanenitrile.

### Step 2

To a 10 ml seal tube under nitrogen atmosphere, (3-(3-(6-amino-8-oxo-7-(4-phenoxyphenyl)-7H-purin-9(8H)-yl)pyrrolidin-1-yl)-3-oxopropanenitrile (100 mg, 0.22 mmole) was dissolved in 1, 4 dioxane (0.4 ml) at rt. To this, 2-methyl-2-morpholinopropanal (207 mg, 1.32 mmole) was added and stirred for 15 minutes. Piperidine (37.5 mg, 0.44 mmole) was added dropwise at rt. The reaction mixture was stirred for 10 h at 80° C. The reaction mixture was diluted by ethyl acetate and washed with water followed by brine solution. The organic layer was dried over sodium sulfate and concentrated to give crude product which was purified by prep HPLC to yield 16 mg of 2-(3-(6-amino-8-oxo-7-(4-phenoxyphenyl)-7H-purin-9(8H)-yl)pyrrolidine-1-carbonyl)-4-methyl-4-morpholinopent-2-enenitrile. LC-MS (ES, m/z): 595.5 [M+H].

### Example 4

### Synthesis of 2-(3-(6-amino-8-oxo-7-(4-phenoxyphenyl)-7H-purin-9(8H)-yl)pyrrolidine-1-carbonyl)-4,4-dimethylpent-2-enenitrile

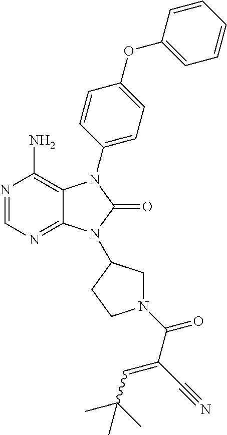 [SMILES: CC(C)(C)C=C(C#N)C(=O)N1CCC(n2c(=O)n(-c3ccc(Oc4ccccc4)cc3)c3c(N)ncnc32)C1]

### Step 1

To a 10 ml vial under nitrogen atmosphere, 2-cyano-4,4-dimethylpent-2-enoic acid, (19.8 mg, 0.13 mmole) was dissolved in DMF (0.2 ml) at rt. To this, HATU (49.4 mg, 0.13 mmole) in DMF (0.2 ml) was added dropwise at 0° C. and the mixture stirred at 0° C. for 30 minutes. 6-Amino-7-(4-phenoxyphenyl)-9-(pyrrolidin-3-yl)-7H-purin-8(9H)-one hydrochloride (50 mg, 0.12 mmole) in DMF (0.2 ml) was added dropwise at 0° C. followed by addition of DIPEA (46.53 mg, 0.36 mmole) at same temperature and the reaction mixture was stirred for 30 minutes at rt. After completion of the reaction, reaction mixture was diluted by ethyl acetate and washed with brine solution followed by saturated sodium bicarbonate solution. The organic layer was dried over sodium sulfate and concentrated to give crude product which was purified using column purification by eluting the compound with 1.2% methanol in CH₂Cl₂ to yield 22 mg of 2-(3-(6-amino-8-oxo-7-(4-phenoxyphenyl)-7H-purin-9(8H)-yl)pyrrolidine-1-carbonyl)-4,4-dimethylpent-2-enenitrile. LC-MS (ES, m/z): 524.3 [M+H].

### Example 5

### Synthesis of (R)-2-(3-(6-amino-8-oxo-7-(4-phenoxyphenyl)-7H-purin-9(8H)-yl)pyrrolidine-1-carbonyl)-4,4-dimethylpent-2-enenitrile 2

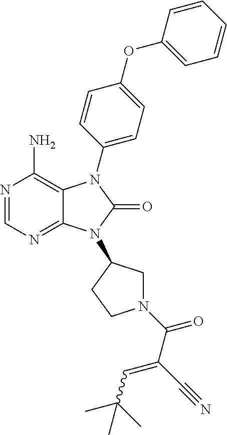 [SMILES: CC(C)(C)C=C(C#N)C(=O)N1CC[C@@H](n2c(=O)n(-c3ccc(Oc4ccccc4)cc3)c3c(N)ncnc32)C1]

Proceeding as described in Example 4 above but substituting 6-amino-7-(4-phenoxyphenyl)-9-(pyrrolidin-3-yl)-7H-purin-8(9H)-one with (R)-6-amino-7-(4-phenoxyphenyl)-9-(pyrrolidin-3-yl)-7H-purin-8(9H)-one, the title compound was prepared.

### Example 6

### Synthesis of (R)-2-(3-(6-amino-8-oxo-7-(4-phenoxyphenyl)-7H-purin-9(8H)-yl)pyrrolidine-1-carbonyl)-4-methyl-4-morpholinopent-2-enenitrile

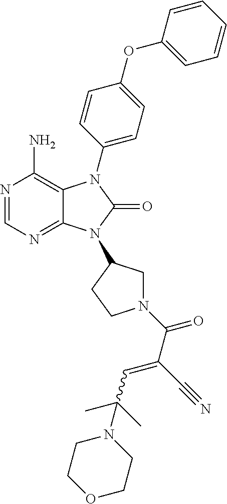

Proceeding as described in Example 3 above but substituting 6-amino-7-(4-phenoxyphenyl)-9-(pyrrolidin-3-yl)-7H-purin-8(9H)-one with (R)-6-amino-7-(4-phenoxyphenyl)-9-(pyrrolidin-3-yl)-7H-purin-8(9H)-one, the title compound was prepared.

### BIOLOGICAL EXAMPLES

### Example 1

### Btk Enzymatic Activity Assay

A Caliper-based kinase assay (Caliper Life Sciences, Hopkinton, Mass.) was used to measure inhibition of Btk kinase activity of a compound of Formula (I). Serial dilutions of test compounds were incubated with human recombinant Btk (2 nM), ATP (40 M) and a phosphoacceptor peptide substrate FAM-GEEPLYWSFPAKKK-NH₂ (1 μM) at room temperature for 3 h. The reaction was then terminated with EDTA, final concentration 20 mM and the phosphorylated reaction product was quantified on a Caliper Desktop Profiler (Caliper LabChip 3000). Percent inhibition was calculated for each compound dilution and the concentration that produced 50% inhibition was calculated. This value is presented as the IC₅₀. The IC₅₀ for a representative no. of compounds of the disclosure are provided below.

| Synthetic | | Synthetic | |
|---|---|---|---|
| Example # | IC₅₀ (nm) | Example # | IC₅₀ (nm) |
| 1 | 0.0097 | 4 | 0.0201 |
| 5 | 0.0201 | 6 | 0.0723 |

### Example 2

### Blockade of CD69 Expression in Whole Blood Samples

Activation of the B cell receptor leads to increased BTK activity, calcium mobilization and B cell activation (see Honigberg L. A., et. al., Proc Natl Acad Sci USA. 107:13075-80. 2010). BTK inhibitors have been shown to block B cell activation as measured by CD69 expression (see Karp, R., et. al., Inhibition of BTK with AVL-292 Translates to Protective Activity in Animal Models of Rheumatoid Arthritis. Inflammation Research Association Meeting, September, 2010). We used expression of CD69 following B cell activation as a measure of BTK activity in whole blood. Aliquots of whole blood were pre-incubated with serial dilutions of test compound for 30 minutes followed by activation with anti-IgM (goat Fab′2, 50 ug/ml). Samples were incubated overnight at 37° C. and then stained with PE labeled anti-CD20 and APC labeled anti-CD69 (BD Pharmingen) for 30 minutes according to the manufacturer's directions. Whole blood was then lysed and cells gated on CD20 expression were quantified for CD 69 expression by FACS. The percent inhibition was calculated based on a DMSO control for no inhibition and plotted as a function of test compound concentration from which an IC₅₀ value was calculated.

### Example 3

### Inhibition of Mouse Collagen-Induced Arthritis

Inhibition of murine collagen-induced arthritis (mCIA) is a standard animal disease model for rheumatoid arthritis. Previous studies have demonstrated that inhibition of BTK is efficacious in blocking mCIA (see Honigberg L. A., et. al., Proc Natl Acad Sci USA. 107:13075-80. 2010). Starting on day 0 DBA/1 mice are injected with an emulsion of Type II collagen in Complete Freund's Adjuvant. Mice are boosted 21 days later to synchronize development of disease. After development of mild disease, animals are enrolled in the study and randomized. Dosing is done oral or intraperitoneal, Q.D. or BID typically for 11 days with test compound or dexamethasone (0.2 mg/kg) as control. One group received vehicle alone.

Clinical scoring (0-4) is based on the extent of swelling and severity of arthritis. Scores for all four paws aresummed for maximum score of 16. (Bolder BioPath, Boulder, Colo.).

### Example 4

### Recovery of Kinase Activity Upon Dialysis

Standard experimental methods to establish reversibility are known in the art. Protein dialysis is one such method. A solution containing a protein kinase that is inhibited by a compound of Formula I may be subjected to extensive dialysis to establish if the kinase inhibitor is reversible. Partial or complete recovery of protein kinase activity over time during dialysis is indicative of reversibility.

### Method:

A compound of Formula I and/or pharmaceutically acceptable salt described herein (1 uM) is added to a solution of protein kinase (50 nM, pre-activated if necessary) in a buffer containing 20 mM Hepes [pH 8.0], 10 mM MgCl₂, 2.5 mM tris(2-carboxyethyl)phosphine (TCEP), 0.25 mg/mL BSA, and 100 uM ATP. After 60 min at rt, the reactions is transferred to a dialysis cassette (0.1-0.5 mL Slide-A-Lyzer, MWCO 10 kDa, Pierce) and dialyzed against 2 L of buffer (20 mM Hepes [pH 8.0], 10 mM MgCl₂, 1 mM DTT) at 4° C. The dialysis buffer is exchanged after 2 h, and then is exchanged every 24 h until the end of the experiment. Aliquots are removed from the dialysis cassettes every 24 h, flash frozen in liquid nitrogen, and subsequently analyzed for protein kinase activity in triplicate. Kinase activity for each sample is normalized to the DMSO control for that time point and expressed as the mean±SD.

Results: Kinase activity recovers from inhibition by reversible kinase inhibitors upon dialysis. Upon extensive dialysis at 4° C. or at room temperature, kinase activity partially or completely recovers in a time-dependent manner from inhibition by an excess (20 equiv, 1.0 uM) of reversible kinase inhibitor.

### Example 5

### Mass Spectral Analysis

A protein kinase that is inhibited by compound of Formula I and/or pharmaceutically acceptable salt may be subjected to mass spectral analysis to assess the formation of permanent, irreversible covalent adducts. Suitable analytical methods to examine intact full protein or peptide fragments generated upon tryptic cleavage of the protein kinase are generally known in the art. Such methods identify permanent, irreversible covalent protein adducts by observing a mass peak that corresponds to the mass of a control sample plus the mass of an irreversible adduct. Two such methods are described below.

### Mass Spectral Analysis of Intact Full Kinase

### Method:

A protein kinase (5 uM) is incubated with a compound of Formula I (25 uM, 5 equiv) for 1 h at room temperature in buffer (20 mM Hepes [pH 8.0], 100 mM NaCl, 10 mM MgCl2). A control sample is also prepared which does not have a compound of Formula I. The reaction is stopped by adding an equal volume of 0.4% formic acid, and the samples are analyzed by liquid chromatography (Microtrap C18 Protein column [Michrom Bioresources], 5% MeCN, 0.2% formic acid, 0.25 mL/min; eluted with 95% MeCN, 0.2% formic acid) and in-line ESI mass spectrometry (LCT Premier, Waters). Molecular masses of the protein kinase and any adducts may be determined with MassLynx deconvolution software.

Results: High-resolution intact mass spectrometry analysis of a kinase that is inhibited by a compound of Formula I will reveal a spectrum similar to the kinase in the absence of inhibitor (e.g. control sample). There will be no formation of a new peak in the mass spectrum corresponding to the molecular mass of the kinase plus the molecular mass of the compound of Formula I. On the basis of this experiment, as can be applied to a compound and/or pharmaceutically acceptable salt as disclosed herein, no permanent, irreversible protein adduct will be apparent to one skilled in the art.

### Mass Spectral Analysis of Kinase Tryptic Digest

### Method:

A protein (10-100 pmols) is incubated with a compound of Formula I and/or pharmaceutically acceptable salt (100-1000 pmols, 10 equiv) for 3 hrs prior to tryptic digestion. Iodoacetamide may be used as the alkylating agent after compound incubation. A control sample is also prepared which does not have the compound of Formula I and/or pharmaceutically acceptable salt. For tryptic digests a 1 ul aliquot (3.3 pmols) is diluted with 10 ul of 0.1% TFA prior to micro C18 Zip Tipping directly onto the MALDI target using alpha cyano-4-hydroxy cinnamic acid as the desorption matrix (5 mg/mol in 0.1% TFA:Acetonitrile 50:50) or Sinapinic acid as the desorption matrix (10 mg/mol in 0.1% TFA:Acetonitrile 50:50).

Results: High-resolution mass spectrometry analysis of the tryptic fragments of a kinase that is inhibited by a compound of Formula I will reveal a spectrum similar to the kinase in the absence of inhibitor (e.g. control sample). There will be no evidence of any modified peptides that are not present in the control sample. On the basis of this experiment, no permanent, irreversible protein adducts will be apparent to one skilled in the art. Cellular assays are also optionally used to assess the inhibiting properties of a compound of Formula I provided herein or embodiments thereof. Cellular assays include cells from any appropriate source, including plant and animal cells (such as mammalian cells). The cellular assays are also optionally conducted in human cells. Cellular assays of BTK inhibition are well known in the art, and include methods in which an inhibitor is delivered into the cell (e.g. by electroporation, passive diffusion, microinjection and the like) and an activity endpoint is measured, such as the amount of phosphorylation of a cellular substrate, the amount of expression of a cellular protein, or some other change in the cellular phenotype known to be affected by the catalytic activity of BTK. For example, phosphorylation of a particular cellular substrate is optionally assessed using a detection antibody specific or the phosphorylated cellular substrate followed by western blotting techniques and visualization using any appropriate means (e.g. fluorescent detection of a fluorescently labeled antibody).

Measuring the reduction in the BTK catalytic activity in the presence of an inhibitor disclosed herein relative to the activity in the absence of the inhibitor is optionally performed using a variety of methods known in the art, such as the assays described in the Examples section below. Other methods for assaying BTK activity are known in the art.

### Example 6

### Determination of Drug-Kinase Residence Time

The following is a protocol that can be used to distinguish whether a compound displays a slow or non-existent dissociation rate from BTK, such as typically would occur if a covalent bond is formed between the compound and the target. The read-out for slow dissociation is the ability of the compound of interest to block binding of a high affinity fluorescent tracer molecule to the kinase active site, as detected using time-resolved fluorescence resonance energy transfer (TR-FRET). The experiment was conducted in a buffer consisting of 50 mM Hepes pH 7.5, 10 mM MgCl₂, 0.01% Triton X-100, and 1 mM EGTA.

The first step of the procedure was incubation of 500 nM BTK (Invitrogen Cat. #PV3587) with 1.5 uM of a compound of Formula (I) and/or pharmaceutically acceptable salt for 30 minutes in a volume of 10 uL. The mixture was then diluted 5-fold by addition of 40 uL of buffer. A 10 uL volume of the diluted kinase/compound solution was then added to a well of a small volume 384 well plate (such as Greiner Cat. #784076). In order to probe for reversibility of the kinase-compound binding interaction, a competition solution containing both a high affinity fluorescent tracer and an antibody coupled to Europium was prepared. For BTK, the competition solution contained 1.5 uM Tracer 178 (Invitrogen Cat. #PV5593), which is a proprietary high affinity ligand for BTK coupled to the fluorophore AlexaFluor 647. The competition solution also contained 80 nM of an Anti-polyhistidine antibody coupled to Europium (Invitrogen Cat. #PV5596) which is designed to bind the polyhistidine purification tag in BTK.

After addition of 10 uL of the competition solution to the Greiner plate, the mixture was incubated for one hour or greater to allow time for dissociation of non-covalent inhibitors and binding of the high affinity tracer. It was expected that covalent and slow dissociating inhibitors will block binding of the tracer while rapidly dissociating non-covalent inhibitors will not. Binding of the tracer to BTK was detected using TR-FRET between the Europium moiety of the Anti-histidine antibody and the AlexaFluor 647 group of Tracer 178. Binding was evaluated using a Perkin Elmer Envision instrument (Model 2101) equipped with filters and mirrors compatible with LANCE-type TR-FRET experiments. Data were plotted at percentage of signal obtained in the absence of competitor compound. The background signal was obtained by omission of BTK from the reaction. If the compound is an irreversible covalent inhibitor, tracer will be completely blocked from binding to the target throughout the entire course of the experiment. If the compound is a reversible covalent inhibitor, the tracer will bind the target as the compound dissociates from the target.

### Example 7

### Reversibility of Binding

The following approach was developed to differentiate compounds that form irreversible bonds with their targets, such as acrylamide compounds, from compound that bind reversibly such as reversible covalent inhibitor. Reactions are prepared with the protein target at a higher concentration than the compounds of interest. Irreversible and reversible covalent compounds bind the target and become depleted from solution. The reactions are then treated with perturbations including both denaturation with 5 M guanidine hydrochloride and digestion with trypsin, disrupting proper folding of the target. It is found that the perturbation returned reversible covalent compounds to solution due to dissociation from the target while irreversible covalent compounds remain bound to the target. The concentration of compound in solution is assessed both preceding and following perturbation using high performance liquid chromatography (HPLC) coupled to tandem mass spectrometry. Compounds of the present invention are expected to depleted from solution in the native state and in solution in the perturbed state indicating that they are reversible.

### Formulation Examples

The following are representative pharmaceutical formulations containing a compound disclosed herein.

### Parenteral Composition

To prepare a parenteral pharmaceutical composition suitable for administration by injection, 100 mg of a water-soluble salt of a compound of disclosed herein is dissolved in 2% HPMC, 1% Tween 80 in DI water, pH 2.2 with MSA, q.s. to at least 20 mg/mL. The mixture is incorporated into a dosage unit form suitable for administration by injection.

### Oral Composition

To prepare a pharmaceutical composition for oral delivery, 400 mg of a compound disclosed herein and the following ingredients are mixed intimately and pressed into single scored tablets.

### Tablet Formulation

The following ingredients are mixed intimately and pressed into single scored tablets.

| | Quantity per tablet |
|---|---|
| Ingredient | mg |
| compound of this disclosure | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per capsule |
|---|---|
| Ingredient | mg |
| compound of this disclosure | 200 |
| lactose spray dried | 148 |
| magnesium stearate | 2 |

### Injectable Formulation

Compound of the disclosure (e.g., compound I) in 2% HPMC, 1% Tween 80 in DI water, pH 2.2 with MSA, q.s. to at least 20 mg/mL

### Inhalation Composition

To prepare a pharmaceutical composition for inhalation delivery, 20 mg of a compound disclosed herein is mixed with 50 mg of anhydrous citric acid and 100 mL of 0.9% sodium chloride solution. The mixture is incorporated into an inhalation delivery unit, such as a nebulizer, which is suitable for inhalation administration.

### Topical Gel Composition

To prepare a pharmaceutical topical gel composition, 100 mg of a compound disclosed herein is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topical administration.

### Ophthalmic Solution Composition

To prepare a pharmaceutical ophthalmic solution composition, 100 mg of a compound disclosed herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Nasal Spray Solution

To prepare a pharmaceutical nasal spray solution, 10 g of a compound disclosed herein is mixed with 30 mL of a 0.05M phosphate buffer solution (pH 4.4). The solution is placed in a nasal administrator designed to deliver 100 μl of spray for each application.

The foregoing disclosure has been described in some detail by way of illustration and example, for purposes of clarity and understanding. It will be obvious to one of skill in the art that changes and modifications may be practiced within the scope of the appended claims.

Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

What is claimed:

1. A compound of Formula (I): 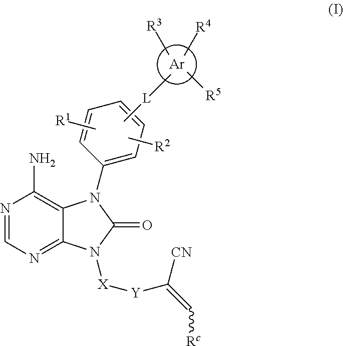
wherein:
L is O, CO, CH₂, S, SO, SO₂, NR, NRCO, CONR, or NRCONR′, where (each R and R′ is independently hydrogen or alkyl);
Ar is aryl, heteroaryl, cycloalkyl or heterocyclyl;
R¹ is hydrogen, alkyl, cyclopropyl, hydroxy, alkoxy, cyano, halo, haloalkyl or haloalkoxy;
R² is hydrogen, alkyl, alkynyl, cyclopropyl, alkylamino, dialkylamino, alkylthio, alkylsulfonyl, carboxy, alkoxycarbonyl, alkylaminosulfonyl, dialkylaminosulfonyl, —CONH₂, alkylaminocarbonyl, dialkylaminocarbonyl, 3-, 4-, or 5-membered heterocylyl, hydroxy, alkoxy, cyano, halo, haloalkyl or haloalkoxy;
R³, R⁴, and R⁵ are independently hydrogen, alkyl, hydroxy, alkoxy, halo, haloalkyl, haloalkoxy, carboxy, alkoxycarbonyl, cyano, —CONH₂, amino, or monosubstituted or disubstituted amino;
X is alkylene, -alkynylene-NR^{a}-, cycloalkylene, -alkylene-O—, -cycloalkylene-NR^{a}—, -(alkylene)-NR^{a}—, -phenylene-NR^{a}— (where each R^{a} is hydrogen, alkyl or cycloalkyl), or 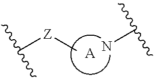
(where Z is bond or alkylene, and ring A is heterocycloamino optionally substituted with one or two substituents independently selected from alkyl, hydroxy, alkoxy, or fluoro);
Y is —CO— or —SO₂—;
R^{c} is alkyl, haloalkoxy, substituted alkyl, cycloalkyl, 1-(alkyleneR^{b})-cycloalkan-1-yl (where R^{b} is amino, alkylamino, dialkylamino, hydroxy, or monocyclic heteroaryl), 1-NR^{d}R^{e}cycloalkan-1-yl (where R^{d} and R^{e} are independently hydrogen, alkyl, or cycloalkyl), or 3 to 6 membered saturated monocyclic heterocyclyl containing one or two heteroatoms selected from N, O, or S and optionally substituted with one or two substituents independently selected from hydroxy, alkoxy, alkyl, fluoro, aminoalkyl, hydroxyalkyl, or alkoxyalkyl;
or a pharmaceutically acceptable salt thereof.

2. The compound or a pharmaceutically acceptable salt of claim 1 wherein:
L is O and is attached at the 4-position of the phenyl ring with the carbon atom of the phenyl ring attached to purinone nitrogen being position 1; and
R¹ and R² are independently hydrogen, alkyl, halo, haloalkyl, or alkoxy.

3. The compound or a pharmaceutically acceptable salt of claim 2 wherein R¹ is hydrogen or halo; and
R² is hydrogen.

4. The compound or a pharmaceutically acceptable salt of claim 2 wherein R¹ and R² are hydrogen.

5. The compound or a pharmaceutically acceptable salt of claim 2 wherein 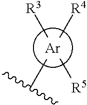
is a ring of formula:

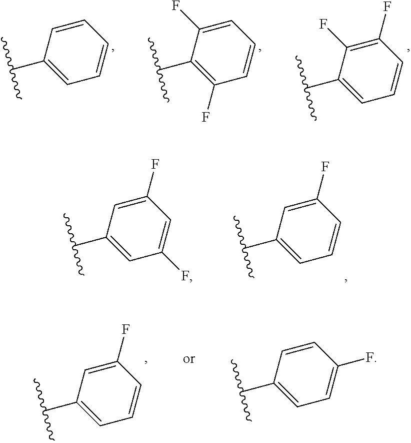

6. The compound or a pharmaceutically acceptable salt of claim 2 wherein 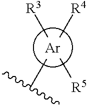
is phenyl.

7. The compound or a pharmaceutically acceptable salt of claim 2 wherein —X— is -cycloalkylene-NR^{a}—, -(alkynylene)-NR^{a}—, -(alkylene)-NR^{a}—, -phenylene-NR^{a}— (where each R^{a} is hydrogen, alkyl or cycloalkyl), or 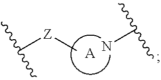
Z is a bond or alkylene;
ring A is heterocycloamino optionally substituted with one or two substituents independently selected from alkyl, hydroxy, alkoxy, or fluoro; and
Y is CO.

8. The compound or a pharmaceutically acceptable salt of claim 2 wherein —X—Y— is:

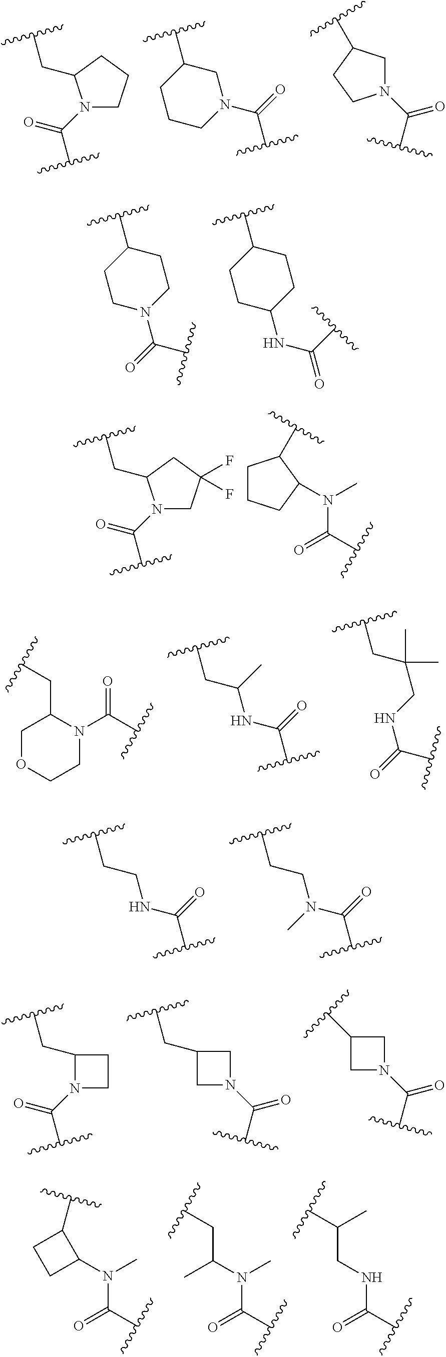

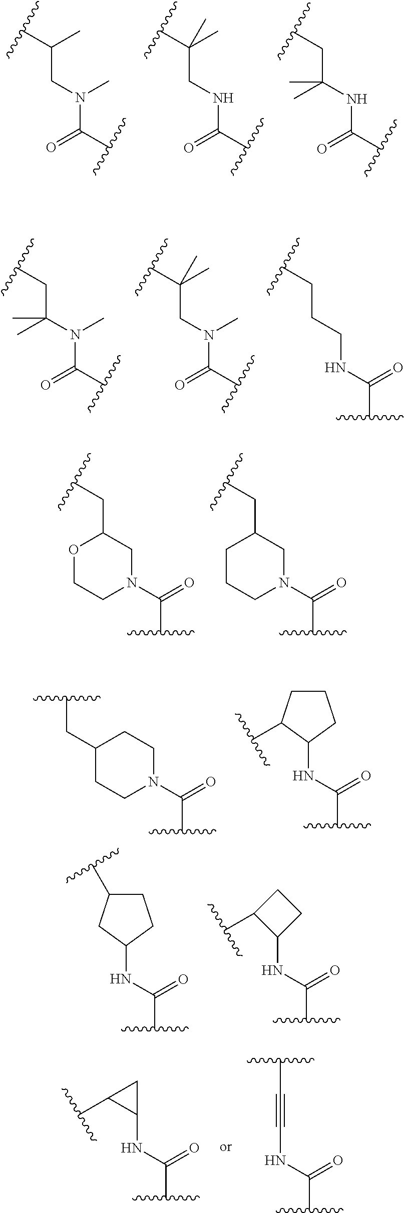

9. The compound or a pharmaceutically acceptable salt of claim 2 wherein —X—Y— is: 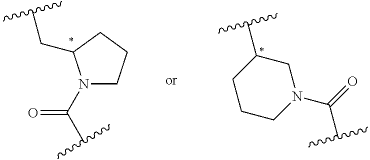 [SMILES: *CC1CCCN1C(*)=O ; *C(=O)N1CCCC(*)C1]
where the stereochemistry at *C is R or S.

10. The compound or a pharmaceutically acceptable salt of claim 9 wherein R^{c} is cycloalkyl.

11. The compound or a pharmaceutically acceptable salt of claim 9 wherein R^{c} is alkyl.

12. The compound or a pharmaceutically acceptable salt of claim 9 wherein R^{c} is alkyl substituted with hydroxyl, alkoxy, —NRR′ (where R is hydrogen, alkyl, alkoxyalkyl, heterocyclyl or cycloalkyl and R′ is hydrogen or alkyl), or heterocyclcyl which is optionally substituted with one or two groups independently selected from alkyl.

13. The compound or a pharmaceutically acceptable salt of claim 9 wherein R^{c} is 3- to 6-membered saturated monocyclic heterocyclyl containing one or two heteroatoms selected from N, O, or S and optionally substituted with one or two substituents selected from hydroxy, alkyl or fluoro.

14. A pharmaceutical composition comprising a compound or a pharmaceutically acceptable salt of claim 1, and a pharmaceutically acceptable excipient.

15. A method of treating an autoimmune disease, inflammatory disease or cancer which method comprises administering to the patient in need thereof, a pharmaceutical composition comprising a compound or a pharmaceutically acceptable salt of claim 1 optionally in combination with one or more anticancer or anti-inflammatory agents.

16. The method of claim 15 wherein the disease is leukemia or lymphoma.

17. The method of claim 15 wherein the leukemia is chosen from chronic lymphocytic leukemia (CLL), small lymphocytic lymphoma (SLL), multiple myeloma, mantle cell lymphoma, and B-cell non-Hodgkin lymphoma.

18. The method of claim 15 wherein the disease is arthritis, lupus, Sjogren's dry eye, non-Sjogren's dry eye, or asthma.
